(19) United States
(12) Patent Application Publication
(10) Pub. No.: US 2015/0133318 A1
(43) Pub. Date: May 14, 2015

(54) ENZYMATIC METHOD TO ENRICH FOR CAPPED RNA, KITS FOR PERFORMING SAME, AND COMPOSITIONS DERIVED THEREFROM

(71) Applicant: University of Massachusetts, Boston, MA (US)

(72) Inventors: Craig C. MELLO, Barrington, RI (US); Weifeng GU, Holden, MA (US)

(73) Assignee: UNIVERSITY OF MASSACHUSETTS, Boston, MA (US)

(21) Appl. No.: 14/260,718

(22) Filed: Apr. 24, 2014

Related U.S. Application Data
(63) Continuation of application No. PCT/US2012/061978, filed on Oct. 25, 2012.
(60) Provisional application No. 61/551,247, filed on Oct. 25, 2011.

(51) Int. Cl. C12N 15/10; G06F 19/22; C12Q 1/68

(52) U.S. Cl. CPC C12N 15/1096; C12Q 1/6876; G06F 19/22
USPC 506/4; 435/91.3; 435/6.1; 506/26; 506/2; 536/23.1; 506/16; 435/196; 702/20

(57) ABSTRACT

The instant invention is based, at least in part, on the identification of novel methods for the enzymatic enrichment of capped RNAs. The invention provides, e.g., methods for enrichment of capped RNAs, kits for making such capped RNAs, and compositions of enriched RNAs or cDNA libraries derived therefrom.

### GOVERNMENT LICENSE RIGHTS

This invention was made with government support under Grant No. NIH 2 RO1 GM058800 and a Grant from the Howard Hughes Medical Institute. The government has certain rights in the invention.

### BACKGROUND

Deep sequencing analysis is increasingly important for providing quantitative assessment of gene expression in both diagnostics and research. To make a high quality cDNA library for deep sequencing of mRNA and other non-structural regulatory RNAs, the RNAs of interest must be enriched from total RNA which contains approximately 98% structural RNAs, including rRNA and tRNA.

Currently, there are two major techniques to enrich for non-structural RNAs: 1) positive selection (e.g., affinity selection of RNAs comprising Poly(A) tails) and 2) negative selection (e.g., using methods which remove rRNAs, which represent approximately 90% of total RNA). Both positive and negative selection techniques employ nucleic acid oligonucleotides anchored to resin to capture the target RNA. Importantly, contamination of samples with fragments of structural RNAs necessitates additional purification steps and greatly reduces the depth of useful sequence data that can be obtained using these existing oligo-based enrichment protocols. Moreover, such additional purification steps are expensive and require large amounts of starting total RNA owing to their low efficiency.

In addition, existing protocols for mRNA cDNA cloning are biased against detection of the 5′ most sequences. Moreover, many genes possess distinct isoforms that employ alternative transcription start sites, and in many cases these isoforms are functionally distinct. Thus, current protocols for mRNA sequencing tend to lack important information about specific mRNA isoforms that are expressed.

The development of a quick and efficient enzymatic enrichment method to enrich for capped RNAs that can be used to obtain deep-sequencing libraries of high quality and that are compatible with liquid-based assays so that they can be processed in a high-throughput manner, would be of great benefit.

### SUMMARY OF THE INVENTION

The instant invention is based, at least in part, on the identification of novel methods for the rapid enzymatic enrichment of capped RNAs.

The instant methods dramatically improve the sensitivity of RNA detection and allow for the rapid detection of the full diversity of alternative 5′ ends in a given transcriptome. Using the instant methods, far less RNA is required than in traditional methods and costly secondary purification steps are eliminated. The method also allows for cloning other capped RNAs, in addition to mRNA (such as primary miRNA or primary piRNA, which are usually not recovered well using existing positive and negative selection methods, if at all). The instant methods also allow for more complete profiling of functional mRNA than methods based on poly(A) selection, because while all functional mRNA contains a cap, functional mRNA may or may not contain a poly(A) tail. In addition, negative selection procedures cannot remove tRNAs, thus removal of tRNA must be performed later, usually by size selection, which also removes some functional capped RNAs of small size.

The instant methods employ a series of enzymatic steps that eliminate contaminating RNA sequences and dramatically enrich for RNAs that contain the nuclease resistant 5′ cap structure that is a feature of mRNAs and several other endogenous RNA species. This procedure dramatically improves the sensitivity of RNA detection and allows the full diversity of alternative 5′ ends to be analyzed. The method also allows for an easy way to process samples in a high-throughput manner.

Specifically, the instant methods comprise a multi-step enzymatic procedure to enrich for capped RNAs. In one step, RNAs which are resistant to 5′ monophosphate-dependent exonuclease are obtained by treatment with 5′-3′ monophosphate-dependent exonuclease. This step removes 28S, 16S, and 5.8S rRNA, but not 5S and mature tRNA, all of which contain 5′ monophosphate. In a subsequent step, the resulting RNAs are treated with a phosphatase to remove exposed 5′ phosphate groups because the 5′-3′ monophosphate-dependent exonuclease cannot destroy all RNAs with 5′ monophosphate, especially highly structured RNAs. This step renders 5S RNA, tRNA, partially degraded rRNAs and triphosphorylated RNA, such as pre-tRNAs, unamenable to subsequent linker ligation, which requires a 5′ phosphate. In the next step, RNAs which are sensitive to decapping enzyme are obtained by treatment with a decapping enzyme to expose the alpha-phosphate in the cap, (Gppp-RNA). The resulting exposed monophosphate from the cap is the only monophosphate available for ligation, the RNAs with monophosphate phosphate (rRNA) or triphoshphate (tRNA) having been destroyed by exonuclease or rendered unamenable to cloning due to dephosphorylation by phophatase in the previous steps.

In one embodiment, the RNA may be ligated with an RNA ligase, which only recognizes 5′ phosphorylated RNA as an acceptor molecule. In one embodiment, cDNA may be obtained by RT PCR using random primers, e.g., chimeric solexa primer-random primers. The resulting cDNA may be subjected to size selection by PAGE gel purification to obtain cDNA within a desired size range. In one embodiment, cDNA may be amplified by PCR using DNA oligonucleotides designed according to the 5′ linker and the primer sequence in the RT reaction. By changing linkers and primers, the above procedure can be applied to many deep-sequencing platforms.

The instant methods provide many advantages over the prior art. As set forth above, most mRNA-sequence analysis uses oligo-based purification to enrich for mRNA using positive or negative selection, both of which involve column purification and require large amounts of total RNA. In contrast, the instant method invention uses an enzymatic approach to enrich for capped RNA, thus avoiding tedious and less efficient column purification procedures. The instant method also results in less RNA degradation than can occur using oligo-based purifications. Moreover, the sequences cloned using the instant methods are anchored at the 5′ end of capped RNAs, thus simplifying the bioinformatics analysis to compare samples.

Using the instant method, it is possible to clone mRNA from very little starting material, e.g., 500 ng or less, an amount lower than can be used with most available techniques. The instant methods can be used, e.g., to sequence mRNA from very small amounts of sample like clinical samples, to sequence partially degraded samples, to sequence pri-miRNA or other capped RNA, to enrich mRNA without column purification (which increases speed and reduces cost) and to process samples in a high-throughput manner using liquid handling robots. The RNAs enriched for using the claimed methods also are more representative of functional mRNAs in eukaryotes than those enriched for using current methods.

Moreover, other protocols which enrich for capped sequences, specifically the CAGE protocol, have a known bias with a nonspecific G at the most 5′ end of the CAGE tags, which is attributed to the template-free 5′-extension during the first-strand cDNA synthesis. This introduces erroneous mapping of CAGE tags. In addition, CAGE uses 5′ linker with random sequence to do 5′ ligation, thus introducing mutations. This does not occur using the instant methods. In addition, in C. elegans, 70% of all functional transcripts get spliced to a leader sequence (SL). The SL is 22 nucleotides long. CAGE can only sequence the first 20 nucleotides of any particular transcript. Thus, the information obtained from CAGE is meaningless 70% of the time in that organism.

Moreover, in contrast to other prior art methods, the instant enzymatic process effectively eliminates rRNA. The combination of phosphatase and tobacco acid phosphatase does not remove ribosomal RNA. rRNA competes with mRNA in the following RT and PCR steps. Most importantly, rRNA can be degraded during the ligation step, which is usually carried out overnight. Even a very tiny amount of degradation, e.g. 1-2%, can give significant contamination with rRNA or other structural RNA, because those RNAs are about 98% of the total RNA.

Of particular importance are the downstream applications made possible by the instant methods. Non-limiting examples include the identification of novel 5′ ends of non-annotated gene transcripts as described in the Examples infra, as well as confirmation of information relating to annotated transcripts, by overlaying reads onto annotated gene transcript information. Moreover, the instant methods can be applied to the identification of novel alternative splice sites (thereby identifying novel alternatively spliced transcripts) at the 5′ end or even within a transcriptome depending on the sequencing platform, and can also be used to quantify mRNA expression levels across different samples in an experiment (e.g., developmental stage transcriptome analysis as described in the Examples section infra). The instant methods can also be applied to identify pri-miRNA and pri/pre-piRNA and other non-polyadenylated but capped RNA. The instant methods can also be applied to samples that have partially degraded due to preparation or storage.

Accordingly, in one aspect, the invention is directed to a method of enriching for capped RNA present in a starting RNA sample containing rRNA comprising: contacting a starting RNA sample 5′ monophosphate-dependent exonuclease to obtain a population of 5′ monophosphate-dependent exonuclease resistant RNAs depleted of 28S, 16S, and 5.8S rRNA; contacting the population of 5′ monophosphate-dependent exonuclease resistant RNAs with at least one phosphatase to obtain a population of phosphatase-resistant RNAs depleted of RNAs having exposed 5′ phosphate groups; and contacting the population of phosphatase-resistant RNAs with a decapping enzyme to obtain a population of RNAs having an exposed alpha-phosphate in the 5′ Gppp cap, to thereby enrich for capped RNA present in a starting RNA sample containing rRNA.

In one embodiment, the starting RNA sample is total cellular RNA. In one embodiment, the starting RNA sample comprises 500 ng or less of RNA.

In one embodiment, the starting RNA sample includes polyA+ and polyA− RNA.

In one embodiment, the starting RNA sample comprises degraded RNA.

In one embodiment, the method further comprises sequencing the population of RNAs having an exposed alpha-phosphate in the 5′ Gppp cap.

In one embodiment, the method further comprises the step of contacting the population of RNAs having an exposed alpha-phosphate in the cap with an RNA ligase to obtain a population of ligated RNAs.

In one embodiment, the method further comprises subjecting the population of ligated RNAs to RT PCR using random primers to obtain a cDNA library.

In one embodiment, the method further comprises subjecting the cDNA library to a size selection procedure.

In one embodiment, the method further comprises amplifying the cDNA library by PCR.

In one embodiment, the method further comprises treating the starting RNA sample with a polynucleotide kinase prior to step a) to phosphorylate the 5′ terminus of degraded RNA.

In one embodiment, the method further comprises sequencing 100 bases or fewer of the cDNA members of the library.

In one embodiment, the method further comprises sequencing between 50 and 125 bases of the cDNA members of the library.

In one embodiment, the invention pertains to a composition obtained using a method of the invention.

In another aspect, the invention pertains to a RNA mixture comprising RNA molecules comprising transcriptional start sites, wherein the RNA molecules comprise monophosphorylated 5′ termini or hydroxyl 5′ termini, wherein the mixture is substantially free of 28S, 16S, and 5.8S ribosomal RNA, RNA with triphosphorylated 5′ termini, and 5′ m7G capped RNA.

In one embodiment, the mixture comprises RNAs substantially free of poly-A at the 3′ terminus.

In one embodiment, the mixture is substantially free of RNA with poly-A at the 3′ terminus.

In one embodiment, the mixture comprises RNA with a size of less than 200 nucleotides. In another embodiment, the mixture comprises RNA with a size of between about 50 nucleotides and about 200 nucleotides. In another embodiment, RNA mixture comprises a substantially pure population of RNA molecules having a size of between about 130 nucleotides and 170 nucleotides.

In one embodiment, the invention pertains to a cDNA library generated from an RNA mixture of the invention.

In another aspect, the invention pertains to a kit comprising: a first component comprising a 5′ monophosphate-dependent exonuclease, a second component comprising a phosphatase, and a third component comprising a decapping enzyme.

In one embodiment, the kit further comprises a control sample.

In one embodiment, the kit further comprises instructions for use of the kit to enrich for capped RNAs.

In another aspect, the invention pertains to methods for identifying transcriptional start (TS) sites.

### BRIEF DESCRIPTION OF THE FIGURES

FIG. 1 is a schematic depicting the steps of an enzymatic method for enhancing capped RNA.

FIG. 2 is a schematic representation of the genomic locus around gene T24H10.6 in C. elegans overlayed with reads obtained from Solexa sequencing. Emb, L1, L3, and YA refer to various developmental stages of C. elegans from which RNA were extracted. The blunt end of each arrow represents the transcriptional start site and the length of the arrow represents the size of the RNA sequenced. The height of an arrow represents the read number on a log scale. Black arrows reflect the lack of trans-splicing, and colored arrows represent a trans-splicing event (i.e., a capped SL leader sequence is spliced with the 5′ end of a transcript, producing the mature capped 5′ end of a transcript). The annotation information of four genes (T24H10.5, T24H10.6, RNAz-513084, and T24H10.3) is shown at the top of the figure. In C. elegans and other organisms, some mRNAs gain a common 5′ capped splice leader (SL) by a process called trans-splicing, in which splicing occurs between two RNA molecules rather than one molecule as in common splicing reactions. Approximately 70% of genes are subjected to this process while the remaining 30% are not and obtain a cap structure directly at their 5′ ends. With respect to the CAGE method, this becomes a serious obstacle given that most sequences contain a common 5′ SL.

FIG. 3 is a schematic representation of the drh-3 (D2005.5) genomic locus overlayed with reads obtained from Solexa sequencing. The details of the figures are as explained for FIG. 2. This particular locus also harbors the genes D2005.3, D2005.8, D2005.4.1, D2005.4.2, and D2005.7.

FIG. 4 is a schematic representation of annotated csr-1 transcripts overlayed with reads obtained from Solexa sequencing. The details of the figures are as explained for FIG. 2. csr-1 has two annotated transcripts.

FIG. 5 is a schematic representation of the annotated pgl-1 transcripts overlayed with reads obtained from Solexa sequencing. The details of the figures are as explained for FIG. 2. pgl1 has three annotated transcripts. The right portion of the figure depicts another gene with two annotated transcripts.

FIG. 6 is a schematic representation of the annotated msp-76 transcript overlayed with reads obtained from Solexa sequencing. The details of the figures are as explained for FIG. 2. msp-76 has one annotated transcript.

FIG. 7 is a schematic representation of annotated K02E2.6 transcripts overlayed with reads obtained from Solexa sequencing. The details of the figures are as explained for FIG. 2. K02E2.6 has two annotated transcripts.

FIG. 8 is a schematic representation of annotated F55C9.3 transcripts overlayed with reads obtained from Solexa sequencing. The details of the figures are as explained for FIG. 2. F55C9.3 has two annotated transcripts.

FIG. 9 (panel above horizontal black line) is a schematic representation of annotated pri-lin4 transcript overlayed with reads obtained from Solexa sequencing. The details of the figure are as explained for FIG. 2. pri-lin-4 is processed to form the pre-lin-4 transcript, which is further processed to form mature lin-4. Both pre-lin-4 and mature lin-4 are annotated at the top of FIG. 9. The panel below the horizontal black line represents small RNA-seq data from gravid adults. The three boxes in the legend represent: cyan, RNA mapped to the Watson strand corresponding to non-21U; purple, RNA mapped to the Crick strand corresponding to non-21U; red, 21U (likely miRNA/21U-RNA) mapped to either the Watson or Crick strand. Most miRNAs begin with U, and some are 21nt or degraded to 21nt. Accordingly, in the 21U-RNA gene loci, red arrows represent 21U-RNA and in miRNA loci, the red arrows represent either miRNA or miRNA degraded to 21 nt. “21U” represents a small RNA of 21nt starting with a 5′U, although not necessarily a 21U-RNA gene. By way of example, 22U, 21G, and 23A would be referred to as “non-21U”.

FIG. 10 (panel above horizontal black line) is a schematic representation of annotated pri-mir-42-43-44-45 cluster overlayed with reads obtained from Solexa sequencing. The details of the figures are as explained for FIG. 2. The various pre-miRNA transcripts are processed to form mature miRNA transcripts. The panel below the horizontal black line represents small RNA-seq data from gravid adults. The three boxes in the legend are as described for FIG. 9.

FIG. 11 is a schematic representation of annotated pri-mir-229-64-65-66 cluster overlayed with reads obtained from Solexa sequencing. The details of the figures are as explained for FIG. 2. The various pre-miRNA transcripts are processed to form mature miRNA transcripts. The panel below the horizontal black line represents small RNA-seq data from gravid adults. The three boxes in the legend are as described for FIG. 9.

FIG. 12 is a schematic representation of annotated pri/pre-21ur-3338 transcripts overlayed with reads obtained from Solexa sequencing. The details of the figures are as explained for FIG. 2. The panel below the horizontal black line represents small RNA-seq data from gravid adults. The three boxes in the legend are as described for FIG. 9.

FIG. 13 is a schematic representation of various other annotated pri-pre-21U-RNA transcripts. The details of the figures are as explained for FIG. 12. For each panel, reads above the dotted black line correspond to CapSeq reads, whereas the read below the line correspond to small RNA-seq reads. The panel below the horizontal black line represents small RNA-seq data from gravid adults. The three boxes in the legend are as described for FIG. 9.

FIG. 14 depicts flowcharts illustrating the CapSeq (left) and CIP-TAP (right) protocols.

FIG. 15 depicts additional capped RNA analyses for mRNAs and miRNAs. Motif analyses of trans-splice sites (upper), upstream antisense csRNAs (middle) and antisense 22G-RNAs (lower). ‘A’ corresponds to either the first nt of the trans-splice acceptor site or the 5′ nt of small RNA read.

FIG. 16 depicts a comparative analysis of RNA-seq protocols that enrich long- and short-capped RNAs (CapSeq and CIP-TAP respectively) and a protocol (CIP-PNK) that clones uncapped short RNAs such as siRNA, piRNA and miRNA species. (A and D) Histograms representing the start sites of mapped reads, as indicated, at a typical protein-coding locus, rps-4 (A), and at a miRNA cluster (D). The height of each histogram bar is proportional to the number of reads sharing the same 5′ nt and the scale (log 2) is shown. Candidate pre-mRNA (A) and pri-miRNA (D) 5′ ends and csRNAs are indicated. Trans-splicing at some genes including rps-4 results in removal of the 5′ UTR of the pre-mRNA, called an “outron”, and the addition of a “Spliced leader”. The major trans-splice site for rps-4 is off the scale as indicated by a break in the bar, and the total number of SL-containing reads is indicated. Two minor trans-splice sites flank the major trans-splice site as indicated by triangles below the CapSeq reads. The outron is indicated by a line below the CapSeq reads; dashes indicate the variable 5′ end of the outron. The blue bars beneath the rps-4 coding sequences in the CIP-TAP and CIP-PNK samples correspond to antisense 22G-RNAs. The asterisks in (D) indicate reads corresponding to miRNA star-strands. (B) Schematic representation of the nucleotide composition around candidate TS sites (the +1 position) identified by CapSeq and CIP-TAP reads (here only sense csRNAs). The nucleotide height (in bits) represents the log 2 ratio of the frequency observed relative to the expected frequency based on genomic nt composition. The enriched YR motif is indicated. (C) Pie charts indicating the relative composition of small RNAs recovered in the CIP-TAP and CIP-PNK samples.

FIG. 17 depicts additional capped RNA analyses for mRNAs and miRNAs. Small RNA reads mapped to rRNA (top) and tRNA (bottom). Genome browser views of CIP-PNK and CIP-TAP reads mapping to a rRNA repeat and a tRNA locus are shown. The size distribution of all rRNA or tRNA CIP-TAP reads is also shown.

FIG. 18 depicts additional capped RNA analyses for mRNAs and miRNAs. (A) Positive correlation of csRNA level and long capped RNA level. The expression levels of csRNA and long capped RNA derived from the same TS sites were compared. (B) Size distribution of sense (upper) or anti-sense (middle) csRNAs and 22G-RNAs (lower).

FIG. 19 depicts additional analysis of 21U-RNA loci. (A) Long capped RNA loci preferentially map 2nt upstream mature 21U-RNAs. Long capped RNA reads that overlap with mature annotated 21U-RNAs were identified, and the relative distance between the 5′ end of the long capped RNA and mature 21U-RNA (x-axis) was plotted against the number of such cases. The negative number represents the position of the 1^{st} nt of the long capped RNA upstream of the 5′ end of mature 21U-RNAs. (B) Size distribution of csRNAs derived from 21U-like loci. (C) Distribution of distance between the 5′ ends of paired 21U-RNAs. (D) YR motif analysis of paired 21U-RNAs. Left, analysis of 1130 pairs each of which has two 21U-RNAs separated by 1nt; right, analysis of 1171 pairs each of which has two 21U-RNAs separated by at least 3 nt.

FIG. 20 depicts an analysis of annotated 21U-RNA loci. (A) Cumulative analysis of the 5′ ends of unique CIP-TAP (orange) and CIP-PNK (blue) sequences with respect to the YRNT motif of a consensus 21U-RNA locus. The scale (log 2) is shown. The red segment starting with U indicates the mature 21U-RNA. (B) Graph showing the length distribution of CIP-TAP/csRNA reads (orange) and 21U-RNA/CIP-PNK reads (blue) mapping to 21U-RNA loci. (C) Graph of csRNA levels plotted against corresponding 21U-RNA levels for each locus. The points in red indicate previously annotated 21A/G/C piRNAs. Points near the X-axis (indicated under the brackets) include 22G-RNAs previously mis-annotated as piRNAs.

FIG. 21 depicts an analysis of 21U-like loci. (A) Genome browser view of a piRNA cluster region from LG1V. The 21ur-747 locus and a nearby 21U-like locus are enlarged beneath the line. The bars indicate the number of reads (linear scale is provided) sharing the corresponding 5′ nt from CIP-TAP (orange) and CIP-PNK (blue), relative to the YRNU motif of 21ur-747 (yellow) and YRNA motif of the 21U-like locus (gray). (B) Schematic representation of the nucleotide composition at canonical 21U-RNA loci (top) and 21U-like loci (bottom). The nucleotide height (in bits) represents the log 2 ratio of the frequency observed relative to the expected frequency based on genomic nt composition. The upstream and TS-site (YR) motifs are indicated. The observed 5′ end of mature 21U-RNAs is indicated by the arrow at the +3 position.

FIG. 22 depicts an analysis of Type-2 21U-RNA loci. (A and B) Comparative analysis of reads from RNA-seq protocols (as indicated). The histograms represent the frequency of reads sharing the same 5′ end at the rps-4 locus (A), and at a non-annotated locus on LGX (B). A log 2 scale is provided for each set of histograms. Mature 21U-RNA reads are enriched in the “oxidized” vs “control” (A) and “PRG-1 1P” vs “Input” (A and B) samples as indicated. Closed triangles point out the position of mature 21U-RNAs enriched in the PRG-1 IP. In (B), the red bars correspond to WAGO-dependent 22G-RNAs likely targeting non-annotated transcripts (dashed arrows). (C) Enlarged regions indicating the precise positions of corresponding CIP-TAP (orange) and PRG-1 IP (blue) reads relative to the YRNU motif indicated below the sequence. Note that one of the TS sites for rps-4 is “RR” rather than “YR”. The open triangles above the CIP-TAP bars point to the likely precursor of the mature 21U-RNAs, which are indicated by the closed triangles below the PRG-1 IP bars.

FIG. 23 depicts a multiple sequence alignment for 6 related 21U-RNA loci on X. (A) Alignment of six homologous 21U-RNA producing loci using CLUSTALW program. Shaded are the 21U-RNA sequences obtained in PRG-1 IP. ‘*’ indicates the identical position. (B) 21U-RNA genomic loci with ‘position’ as the start site, ‘reads’ as the PRG-1 IP read number, and ‘score’ as 21U-RNA motif score. Column 7 and 8 are for existence of the −2 csRNA reads from CIP-TAP and −2 CapSeq reads.

FIG. 24 shows the results of CapSeq analysis of mouse testis RNA. (A and B) Browser views of representative protein-coding and piRNA cluster regions are shown. The histograms (log 2 scale) represent the frequency of reads sharing the same 5′ ends from CapSeq or Milli IP (Robine et al., Curr Biol, 19: 2066-2076, 2009) as indicated. Bidirectional reads were observed around the TS sites of Gpatch4. (C) Schematic representation of the nucleotide composition around candidate TS sites (YR). The nucleotide height represents the log 2 ratio of the frequency observed relative to the expected frequency based on genomic nt composition.

FIG. 25 depicts a model for the biogenesis of 21U-RNA. Arrows indicate TS sites of Type 1 and Type 2 piRNA loci. Many Type 2 piRNA loci correspond to protein-coding genes, which generate both sense and anti-sense csRNAs as well as longer capped sense RNAs. Both Type 1 and Type 2 loci express csRNAs that are processed into piRNAs by removal of the cap and 2 nt. A csRNA with +3U is required for efficiently processing or loading onto PRG-1 or to form a stable Piwi/piRNA complex.

### DETAILED DESCRIPTION

### Definitions

Before further description of the invention, certain definitions are included here:

As used herein, “nucleic acid” refers to DNA, RNA and derivatives thereof. The terms “DNA” and “RNA” refer to deoxyribonucleic acid and ribonucleic acid, respectively. The term “mRNA” refers to messenger RNA. The term rRNA refers to ribosomal RNA. The term “tRNA” refers to transfer RNA.

“polyA+RNA” refers to RNA comprising a poly(A) tail which consists of multiple adenosine monophosphates; in other words, it is a stretch of RNA that has only adenine bases at the 3′ end of an RNA molecule. “polyA-RNA” refers to RNA molecules lacking a polyA tail.

The term “cap” with respect to RNAs refers to the cap found on the 5′ end of an mRNA molecule which consists of a guanine nucleotide connected to the mRNA via an unusual 5′ to 5′ triphosphate linkage. This guanosine is methylated on the 7 position directly after capping in vivo by a methyl transferase. It is also referred to as a 7-methylguanylate cap, abbreviated m⁷G or m⁷Gppp. Capped RNA refers to an RNA comprising a 5′ cap and is also referred to as Gppp-RNA.

As used herein, the term MicroRNA (miRNA) refers to RNA molecules that are processed from small hairpin RNA (shRNA) precursors that are produced from miRNA genes. miRNAs are 21-23 nucleotides in length and through the RNA-induced silencing complex they target and silence mRNAs containing imperfectly complementary sequence. Animal miRNAs are initially transcribed as part of one arm of an 80 nucleotide RNA stem-loop that in turn forms part of a several hundred nucleotides long miRNA precursor termed a primary miRNA (pri-miRNA). Animal miRNAs are initially transcribed as pri-miRNA by DNA-dependent RNA polymerase II, then processed by Drosha as pre-miRNA, a stem-loop structure molecule, and finally cut by Dicer to form mature miRNA.

The term Piwi-interacting RNAs (piRNA) refers to small RNA species that are processed from single-stranded precursor RNAs. They are 21-35 nucleotides in length and form complexes with the piwi protein. Pri-piRNAs are long primary transcripts of piRNA molecules.

As used herein, the term “21U” refers to a 21 nt transcript starting with U (i.e., not limited to the 21U-RNA genes depicted in the Figures). However, “21U-RNA” or a gene name starting with “21ur” followed by a suffix is intended to refer to a 21U-RNA gene.

Where a method disclosed herein refers to “amplifying” a nucleic acid, the term “amplifying” refers to a process in which the nucleic acid is exposed to at least one round of extension, replication, or transcription in order to increase (e.g., exponentially increase) the number of copies (including complimentary copies) of the nucleic acid. The process can be iterative including multiple rounds of extension, replication, or transcription. Various nucleic acid amplification techniques are known in the art, such as PCR amplification or rolling circle amplification.

A “primer” as used herein refers to a nucleic acid that is capable of hybridizing to a complimentary nucleic acid sequence in order to facilitate enzymatic extension, replication or transcription.

As used herein the term “transcriptional start site” refers to the site of an mRNA molecule at which transcription begins.

As used herein the term “exposed alpha-phosphate” refers to the 5′ phosphate which was linked to the guanine nucleotide in the cap structure.

As used herein, the term “ligated RNAs” refers to RNAs which have been ligated with at 5′ phosphoryl-terminus through the formation of a 3′→5′ phosphodiester bond, with hydrolysis of ATP to AMP and PPᵢ.

As used herein, the term “RT-PCR” refers to Reverse transcription polymerase chain reaction. RT-PCR is a variant of polymerase chain reaction (PCR), a laboratory technique commonly used in molecular biology to generate many copies of a DNA sequence. In RT-PCR an RNA strand is first reverse transcribed into its DNA complement (complementary DNA, or cDNA) using the enzyme reverse transcriptase, and the resulting cDNA is amplified using traditional PCR or real-time PCR.

As used herein, the term PCR refers to a method of amplifying nucleic acid molecules which relies on thermal cycling, consisting of cycles of repeated heating and cooling of the reaction for DNA melting and enzymatic replication of the DNA. Primers (short DNA fragments) containing sequences complementary to the target region along with a DNA polymerase are key components to enable selective and repeated amplification. As PCR progresses, the DNA generated is itself used as a template for replication, setting in motion a chain reaction in which the DNA template is exponentially amplified.

A cDNA library is a combination of cloned cDNA (complementary DNA) fragments inserted into a collection of host cells, which together constitute some portion of the transcriptome of the organism. A cDNA library, in the context of the present invention, can also refer to the product obtained from reverse transcription of a RNA mixture comprising RNA molecules comprising transcriptional start sites, wherein the RNA molecules comprise monophosphorylated 5′ termini or hydroxyl 5′ termini, wherein the mixture is substantially free of 28S, 16S, and 5.8S ribosomal RNA, RNA with triphosphorylated 5′ termini, and 5′ m7G capped RNA.

As used herein, the term “random primers” refers to short segments of single-stranded DNA (ssDNA) called oligonucleotides, or oligos for short. These oligos are only 8 nucleotides long (octamers) and they consist of every possible combination of bases which means there must be 4⁸=65,536 different combinations in the mixture. Because every possible octamer is present, these primers can bind to any section of DNA.

As used herein, the term “size selection” refers to subjecting a population of nucleic acid molecules to a process that allows for selection of molecules having a desired size range. One such process is gel electrophoresis, in which smaller molecules move through the gel more quickly than larger molecules. The inclusion of reference markers allows for selection of molecules of a specific size range.

The term “deep sequencing” refers to a method of sequencing a plurality of nucleic acids in parallel. See e.g., Bentley et al., Nature, 456:53-59, 2008. Sequencing depth refers to the total number of all the sequences reads or base pairs represented in a single sequencing experiment or series of experiments.

As used herein, the term “tag” refers to a non-target nucleic acid component that provides a means of addressing a nucleic acid fragment to which it is joined. For example, in preferred embodiments, a tag comprises a nucleotide sequence that allows for identifying, recognition, and/or molecular or biochemical manipulation of the DNA to which the tag is attached (e.g., by providing a site for annealing an oligonucleotide, such as a primer for extension by a DNA polymerase, or an oligonucleotide for capture or for a ligation reaction). The process of adding the tag to a DNA molecule can also be referred to as “tagging” and DNA that undergoes tagging or that contains a tag is referred to as “tagged” (e.g., “tagged DNA”). A “sequencing tag domain” or a “sequencing tag” refers to a tag domain that exhibits a sequence for the purpose of facilitating sequencing of the DNA fragment on which the tag is present.

As used herein, the term “read” refers to a sequence of nucleotides within a cDNA (i.e, a short subsequence of a cDNA sequence), where said cDNA is a copy of all or a portion of an RNA molecule enriched according to the methods of the invention for rapid enzymatic enhancement of capped RNAs. Exemplary reads are of cDNA sequences generated according to the CapSeq methods of the invention. In exemplary embodiments, a “read” comprises between about 50 and about 125 nucleotides, preferably 100 nucleotides or fewer, e.g., 20, 30, 40, 50, 60 70, 80, 90 or 100 nucleotides. In exemplary embodiments, a “read” comprises about 50-100 nucleotides, for example, about 70-90 nucleotides, e.g., 70-80 or 80-90 nucleotides. A read can be a sequence derived directly from a cDNA or can result from analysis and/or modification, e.g., trimming, of a cDNA sequence. A “read” generated according to the methods of the invention for rapid enzymatic enhancement of capped RNAs or CapSeq protocols of the invention can also be referred to as a “5′ sequence tag.”

### I. Methods of Enriching for Capped RNAs

A. Starting RNA Samples

Starting RNA samples for enrichment of capped sequences can be obtained from a population of cells. Such starting RNA samples comprise a population of RNA molecules, i.e., a plurality of RNA molecules. Exemplary cells from which starting RNA samples can be obtained include prokaryotic and eukaryotic cells and include, for example, bacterial cells, animal cells, fungal cells and plant cells. In one embodiment, the cell is a mammalian cell, e.g., a human cell. In one embodiment, the cell is obtained from an organ or an organism. In another embodiment, the starting material can be an organism (e.g., C. elegans) or lysate thereof. The cell may be from a clinical sample, e.g., may be obtained from a subject that suffers from a disease or disorder.

The present invention is not limited by the nature of the sample. Samples include, but are not limited to, cell lysates, provided that nucleases that can affect nucleic acids of interest are removed or inhibited in said lysates, mixtures of nucleic acids (e.g., unpurified, partially purified, etc.), environmental samples, etc. Samples can comprise nucleic acids from a single-cell organism or, if an organism comprises multiple cells, from one or more cells. Samples comprising nucleic acids from multiple cells can comprise cells from one or more types of cells, including cells from different organisms, and/or different cells from the same organism. The present invention finds use with prokaryotic nucleic acid molecules, eukaryotic nucleic acid molecules, or with a mixture of both eukaryotic and prokaryotic nucleic acid molecules. Prokaryotic mRNAs lack caps.

In some preferred embodiments of the present invention, the sample comprises (or contains) RNA. In one embodiment, the starting RNA sample comprises total cellular RNA (e.g., structural RNA (such as rRNA and tRNA) as well as mRNA) and includes both polyA+ and polyA− RNA. RNA may be obtained from a cell using techniques known in the art. Typically, the cell is lysed and a starting RNA sample is obtained using known RNA isolation techniques. Exemplary methods include, for example, a MasterPure RNA Purification Kit, an ArrayPure Nano-Scale RNA Purification Kit, or a MasterPure Yeast RNA Purification Kit (all of which are from EPICENTRE), or using a kit from another commercial source, or using a “home-brew” method known in the art. Alternatively, in some embodiments a sample of the invention that comprises or contains RNA can contain a subfraction of total RNA obtained by any method known in the art, such as, but without limitation, a subfraction based on size (e.g., by purification on an agarose or polyacrylamide gel, or by column purification, including by HPLC), or a subfraction obtained by salt precipitation (e.g., using precipitation with 0.5-2.5 M LiCl (Barlow et al., Biochem. Biophys. Res. Comm. 13: 61, 1963); Cathala et al, DNA 2: 329, 1983) or 2.5 M ammonium acetate). In some embodiments, a sample comprising RNA can also contain DNA.

One preferred method of the invention comprises a method for enriching for an RNA having a 5′-cap in a biological sample comprising prokaryotic RNA, eukaryotic RNA or both prokaryotic and eukaryotic RNA and at least one undesired nucleic acid, e.g., structural RNA.

In different embodiments of this method, the RNA having a 5′-cap is selected from the group consisting of: (i) prokaryotic mRNA; (ii) eukaryotic mRNA, including polyadenylated and non-polyadenylated eukaryotic mRNA; (iii) a mixture of both prokaryotic and eukaryotic mRNA; (iv) eukaryotic snRNA; (v) eukaryotic pre-micro RNA; and (vi) prokaryotic or eukaryotic primary RNA transcripts of unknown function.

The instant enzymatic method of enriching for capped RNA requires only small amounts of RNA, e.g., 5000 ng or less of starting RNA. In one embodiment, a starting RNA sample comprises 5000 ng or less RNA, 2500 ng or less RNA, 1000 ng or less RNA, 750 ng or less RNA, 500 ng or less RNA, 250 ng or less RNA, or 100 ng or less RNA.

The instant enzymatic method can also be used to enrich for capped RNAs in a degraded preparation. In one embodiment, a starting RNA population comprises degraded RNA.

In one embodiment, a starting RNA population for use in the instant methods comprises mRNA as well as other capped but less abundant pri-miRNA and/or pri-piRNAs or other non-coding regulatory RNAs.

Starting RNA samples may or may not undergo further manipulation steps prior to the enzymatic enrichment of capped RNAs set forth below. For example, if a large amount of degraded RNA is present in the starting RNA sample, the starting RNA sample can be treated with a polynucleotide kinase prior to enzymatic enrichment for capped RNAs to phosphorylate the 5′ end of degraded RNA. This phosphorylation will render RNA degradation fragments with 5′ OH groups sensitive to 5′ monophosphate-dependent exonuclease.

B. Enzymatic Enrichment of Capped RNAs

The abundance of structural RNAs in the cell creates a challenge for monitoring the expression of Pol II RNA transcripts. Roughly 95% to 98% of cellular transcripts are structural RNAs (rRNA and tRNA) that can result in a high level of noise in RNA-seq experiments. These structural transcripts are 5′-monophosphorylated, and most are sensitive to enzymatic treatment with 5′-to-3′ nucleases, such as Terminator™ exonuclease. Nevertheless, 5′ to 3′ exonuclease treatments cannot completely remove a large molar excess (relative to mRNA) of 5S RNA, tRNA and other partially-degraded rRNA fragments. The presence of these structural RNA fragments has necessitated the use of tedious, inefficient and costly hybridization-based methods to enrich for mRNAs prior to cDNA library construction (Vivancos et al., Genome Res, 20: 989-999, 2010; Wang et al., Nat Rev Genet, 10: 57-63, 2009).

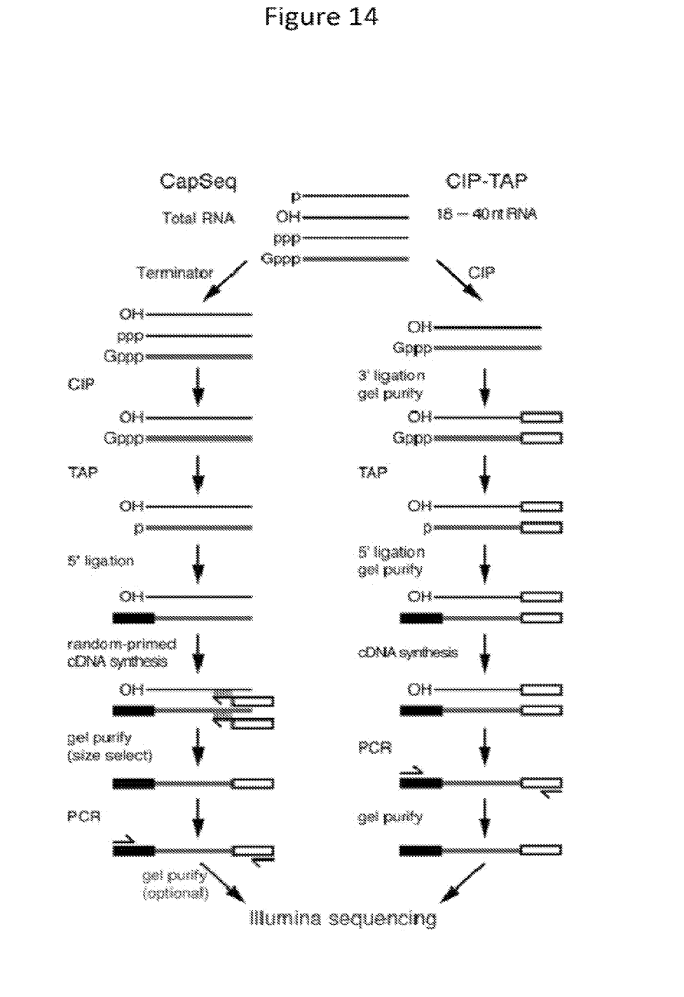

The present invention features a purely enzymatic approach that facilitates cloning the 5′ ends of Pol II transcripts from small quantities of tissue. The presence of a 5′ phosphate is required for ligation mediated by RNA ligase. Removal of the phosphate from 5S RNA, tRNA and partially degraded rRNA, for example, after 5′ monophosphate-dependent (or 5′ to 3′) exonuclease, e.g., Terminator, treatment can prevent cloning of such RNAs during library construction. Therefore, phosphatase, e.g., calf intestine phosphatase (CIP) was used to remove 5′ phosphates from RNA samples, following digestion with 5′ monophosphate-dependent (or 5′ to 3′) exonuclease (FIG. 14). To remove genomic DNA contamination, phosphatase-treatment was carried out together with DNase I (see Experimental Procedures following Example 18). Pol II products are protected from both exonuclease and phosphatase by a 5′ cap structure, but also lack a 5′ phosphate necessary for cloning. Therefore, RNA samples were treated with a decapping enzyme, e.g., tobacco acid pyrophosphatase (TAP) to remove the 5′ cap, exposing a 5′-monophosphate on Pol II transcripts.

The basic steps of the protocol for enzymatic enrichment of capped RNAs are set forth below. As is set forth herein, it will be understood that additional steps may be performed before or after these steps. In addition, it is noted that manufacturers of enzymes that may be used in this method provide suggested concentrations of enzymes and reaction conditions (including, e.g., appropriate buffers and incubation temperatures and times). It will be understood that while the appended Examples set forth the specific experimental conditions used to demonstrate that the instant method enriches for several different types of capped RNAs, the specific enzymes and reaction conditions used may be varied by the skilled artisan, e.g., based on manufacturers' instructions.

1. 5′ Monophosphate-Dependent Exonuclease

In one embodiment, a starting RNA sample is contacted with a 5′ monophosphate-dependent exonuclease to obtain a population of 5′ monophosphate-dependent exonuclease resistant RNAs depleted of 28S, 16S, and 5.8S rRNA.

One exemplary such 5′ monophosphate-dependent exonuclease is the terminator exonuclease commercially available from Epicenter. Another exemplary such nuclease is XRN-1 which is commercially available from NEB.

One preferred method of the invention comprises a method for enriching for an RNA having a 5′-cap in a biological sample comprising prokaryotic RNA, eukaryotic RNA or both prokaryotic and eukaryotic RNA and at least one undesired nucleic acid, the method comprising treating the sample with purified 5′ exoribonuclease under conditions in which the 5′ exoribonuclease is active and for sufficient time so that the undesired nucleic acid is digested and the sample is enriched for RNA having a 5′-cap. The enzymatic reaction can be carried out until the starting sample is substantially depleted of 28S, 16S, and 5.8S rRNA. This can be readily determined by one of ordinary skill in the art by testing aliquots of reaction mixture for the presence of the molecules to be depleted. Enzyme activity of a 5′ exonuclease can be measured using a number of different methods. Without limitation, suitable methods that can be used for assaying activity and determining relative activity using RNA substrates with a 5′-triphosphate, a 5′-cap, or a 5′-monophosphate are described by Stevens and Poole (J. Biol. Chem. 270: 16063, 1995).

The 5′ monophosphate-dependent exonuclease reaction conditions can be optimized by titrating enzyme or substrate RNA, or by performing time course experiments. The time course for digestion can be performed using total RNA as starting material, followed by visualization on a 5% denaturing PAGE gel with SYBR gold or ethidium bromide staining. In the event that the reaction has reached completion, 26S and 18S rRNAs should no longer be visible on the gel. To ensure that capped RNAs are intact, a radio-labeled 5′ capped RNA made by in vitro transcription using methods known in the art can be added to the digestion mixture and monitored using a PAGE gel. Alternatively, the reaction could be monitored using real-time PCR. In this case, cDNA can be produced using random primers in a reverse transcriptase reaction, followed by real-time PCR to compare the amounts of rRNA and mRNA.

2. Phosphatase

The 5′ monophosphate-dependent exonuclease resistant RNAs are subsequently contacted with at least one phosphatase to obtain a population of phosphatase-resistant RNAs depleted of RNAs having exposed 5′ phosphate groups.

One exemplary such phosphatase is Alkaline Phosphatase, e.g., calf intestinal phosphatase (CIP) which catalyzes the removal of 5′ phosphate groups from DNA, RNA, ribo- and deoxyribonucleoside triphosphates. CIP is commercially available from NEB, Promega, Invitrogen, and other sources, such as Thermo Scientific and Affymetrix.

This step removes all exposed phosphates. Other phosphatases that can be employed include, e.g., bacterial alkaline phosphatase and HK phosphatase, which are also commercially available. Other substitutes include APex™ heat-labile alkaline phosphatase (Epicenter) and TSAP alkaline phosphatase (Promega).

The enzymatic reaction can be carried out until the sample being treated is substantially depleted of exposed phosphates, e.g. including 5S RNA, tRNA, partially degraded rRNAs and triphosphorylated RNA like pre-tRNAs. Phosphatases are very robust enzymes and it is rare for the reaction not to go to completion.

The phosphatase treatment step can be monitored by adding a 5′ radio-labeled substrate to the reaction, followed by monitoring with PAGE gel analysis using art-recognized methods.

In one embodiment, during phosphatase digestion, DNase I may optionally be added to remove contaminated genomic DNA. Although this step is not critical, it can increase the quality of the resulting library.

3. Decapping Enzyme

The phosphatase-treated RNAs are subsequently contacted with a decapping enzyme. One such decapping enzyme, Tobacco Acid Pyrophosphatase (TAP), hydrolyzes the phosphoric acid anhydride bonds in the triphosphate bridge of the cap structure found in most eukaryotic mRNA, releasing the cap nucleoside and generating a 5′-monophosphorylated terminus on the RNA molecule. Similarly, TAP digests the triphosphate group at the 5′ end of prokaryotic transcripts, generating an RNA molecule with a 5′-monophosphorylated terminus. TAP is commercially available from many sources (e.g., Epicentre). Other enzymes that catalyze cap processing to GDP and monophosphates can also be substituted for TAP, e.g., human Dcp2 and yeast Dcp1.

The enzymatic reaction can be carried out until the RNAs are substantially enriched for the presence of a 5′-monophosphorylated terminus on the RNA molecules. This can be readily determined by one of ordinary skill in the art by testing aliquots of reaction mixture for the presence of the desired terminus or for removal of caps.

The decapping step can be monitored by adding a 5′ radiolabeled capped substrate made by in vitro transcription to the reaction, followed by sample treatment with TAP and CIP simultaneously. If the reaction has proceeded to completion, the labeled phosphate will be released from the RNA. This can be easily quantified using a PAGE gel with art-recognized methods.

This three step enzymatic process results in a population of 5′ monophosphorylated RNAs that are substantially enriched for (formerly) capped RNAs. The resulting population may optionally be subject to further manipulation steps, e.g., as set forth below.

C. Ligation or Dephosphorylation

In one embodiment, the population of de-capped RNAs having an exposed alpha-phosphate obtained using the enzymatic protocol set forth herein is contacted with an RNA ligase to obtain a population of ligated RNAs. In one embodiment, the ligase is a T4 RNA ligase. Ligases (e.g., T4 ligases) are commercially available from many resources, e.g., Takara, Ambion, Invitrogen, or NEB.

In another embodiment, the population of RNAs having an exposed alpha-phosphate obtained using the enzymatic protocol set forth herein is dephosphorylated by contacting the population with a phosphatase, e.g., APex™ Heat-Labile Alkaline Phosphatase, for end-labeling.

D. cDNA Libraries

In one embodiment, the population of ligated RNAs is subjected to RT PCR using random primers to obtain a cDNA library. Methods for using RT-PCR to obtain cDNA libraries are well known in the art. In one embodiment, the cDNA library may be size selected, e.g., using gel electrophoresis, to obtain nucleic acid molecules of a desired size range.

In one embodiment, the cDNA library may be amplified by PCR using techniques well known in the art. It is also noted that amplification-free protocols have been developed so that amplification is not required prior to sequencing. For example, commercially available single-molecule sequencing technologies, e.g., from Helicos and Pacific Biosciences do not require PCR amplification before sequencing. The Helicos system can even directly sequence RNAs without cDNA library construction. Therefore, in one embodiment, RNAs may be directly sequenced.

In an exemplary embodiment, the enriched RNA populations can be used to generate libraries, referred to herein as “CapSeq libraries.” In this embodiment, a 5′-adapter is ligated and first-strand cDNA is synthesized using a 3′-adapter linked to a random octamer, thus avoiding a second ligation step. Second-strand cDNA is amplified from the first strand cDNA using linear PCR with a primer mapped to the 5′ linker, and the first- and second-strand cDNAs are size-fractionated. Finally, libraries are amplified using PCR and subject to deep-sequencing. This procedure can typically be performed with 0.5-2 μg of total RNA, and the entire process can easily be completed within e.g., about 2 days.

This “CapSeq” protocol provides a convenient approach for 5′-oriented RNA sequencing. The protocol can be performed on small quantities of total RNA without the need for affinity- or hybridization-based purification steps.

E. Sequencing

In one embodiment, a population of molecules obtained using the instant methods, e.g., RNA or cDNA derived therefrom, may be sequenced. The RNAs obtained using the instant cap-enrichment process are particularly well suited for analysis of the transcriptome and, in one embodiment, RNA or cDNA molecules synthesized from these cap-enriched RNAs can be sequenced using known methods.

Illustrative non-limiting examples of nucleic acid sequencing techniques include, but are not limited to, chain terminator (Sanger) sequencing and dye terminator sequencing. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296, 2009; each herein incorporated by reference in their entirety). Most current methods describe the use of next-generation sequencing technology for de novo sequencing of whole genomes to determine the primary nucleic acid sequence of an organism. In addition, the instant methods are particularly well suited to targeted re-sequencing (deep sequencing) and allow for sensitive mutation detection within a population of wild-type sequence. Recent publications describing the use of bar code primer sequences permit the simultaneous sequencing of multiple samples during a typical sequencing run including, for example: Margulies et al., Nature, 437: 376-80, 2005; Mikkelsen et al., Nature, 448: 553-60, 2007; McLaughlin et al., ASHG Annual Meeting, 2007; Shendure et al., Science, 309: 1728-32, 2005; Harris et al., Science, 320: 106-9, 2008; Simen et al., 16th International HIV Drug Resistance Workshop, Barbados, 2007; Thomas et al., Nature Med., 12: 852-855, 2006; Mitsuya et al., J. Vir., 82: 10747-10755, 2008; Binladen et al., PLoS ONE, 2: e197, 2007; and Hoffmann et al., Nuc. Acids Res., 35: e91, 2007, all of which are herein incorporated by reference.

Next-generation sequencing (NGS) methods share the common feature of massively parallel, high-throughput strategies, with the goal of lower costs in comparison to older sequencing methods. These methods are particularly well suited to deep sequencing. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A—addition facilitates addition of T—overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al., J Am Chem Soc. 2006 Feb. 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined.

HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety) is the first commercialized single-molecule sequencing platform. This method does not require clonal amplification. Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run. Other emerging single molecule sequencing methods real-time sequencing by synthesis using a VisiGen platform (Voelkerding et al., Clinical Chem., 55: 641-658, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition. Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10.times.10.sup.-21 L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In one embodiment, libraries of tagged DNA fragments can be made from target DNA for use in, e.g., deep sequencing and amplification methods using transposon compositions. Exemplary such methods are known in the art (see, e.g., US application 20100120098. For example, linear ssDNA fragments or tagged circular ssDNA fragments (and amplification products thereof) from target DNA comprising any dsDNA of interest (including double-stranded cDNA prepared from RNA) for analysis.

### II. Uses

A. Identification of 5′ Ends

In an exemplary aspect, the instant methods can be used to effectively clone the 5′ ends of as of yet unannotated transcripts (e.g., pri-lin-4 as demonstrated in the instant Examples). Thus, the instant methods effectively identify 5′ ends and allow analysis of the transcriptome to map 5′ start sites. In addition, as will be recognized by one of ordinary skill in the art, by anchoring at the 5′ end and using gene specific primers, the instant methods can be used to determine, e.g., which start sites correlate with a given isoform of a molecule. Moreover, whether the newly identified 5′ ends actually correspond to transcripts of the gene being studied (e.g., pri-lin-4) can be assessed with art-recognized techniques, e.g., Northern blot analysis to confirm the size of the transcript. In the event of low expression of the transcript, another option is RT-PCR, followed by nested PCR with gene-specific primers. The latter is the routine process of 5′ RACE or 3′RACE followed by normal sequencing to obtain the ends of a gene.

B. Identifying Argonaute (AGO)-Associated Small RNAs

The methods of the invention can be used to explore the expression and biogenesis of small RNAs in a model organism, in particular, small RNAs believed to be involved in gene regulation. Of particular interest are genes that associate with Argonaute (AGO) proteins, such proteins playing a known role in sequence-directed gene regulation by association with a variety of classes of small RNAs.

The Argonaute (AGO) proteins associate with small RNAs to form sequence-directed gene regulatory complexes that are deeply conserved in eukaryotes (Hutvagner et al., Nat Rev Mol Cell Biol, 9: 22-32, 2008). Most organisms encode multiple functionally-distinct AGO family members. These AGOs are loaded with a diversity of small RNA cofactors, produced through a similarly diverse repertoire of small-RNA biogenesis mechanisms (Siomi et al., Nature, 457: 396-404, 2009). AGO-associated small RNAs include micro (mi) RNAs and short-interfering (si) RNAs that are processed from doublestranded (ds) RNA precursors by the RNase III-related enzyme Dicer (Bernstein et al., Nature, 409: 363-366, 2001).

In some organisms, AGO-associated small-RNA species are produced, independent of Dicer, by RNA-dependent RNA Polymerase (RdRP) (Gu et al., Mol Cell., 36: 231-244, 2009; Pak et al., Science, 315: 241-244, 2007; Sijen et al., Science, 315: 244-247, 2007). Still others, such as the piRNAs of animals, are produced, at least in part, through Pol II transcription (Cecere et al., Mol Cell, 47(5): 734-45, 2012; Saito et al., Nature, 461: 1296-1299, 2009).

1. Piwi-Interacting RNAs

piRNAs are Dicer-independent small RNAs that interact with AGOs related to Drosophila Piwi (Aravin et al., Nature, 442: 203-207, 2006; Girard et al., Nature, 442: 199-202, 2006; Grivna et al., Genes Dev, 20: 1709-1714, 2006; Lau et al., Science, 313: 363-367, 2006; Ruby et al., Cell, 127: 1193-1207, 2006).

Many piRNA species originate from large genomic clusters and direct Piwi-dependent transposon silencing, heterochromatin modification and germ cell maintenance (Aravin et al., Science, 318: 761-764, 2007a; Batista et al., Mol Cell., 31: 67-78, 2008; Brennecke et al., Cell, 128: 1089-1103, 2007; Das et al., Mol Cell., 31: 79-90, 2008; Lin, Science, 316: 397, 2007). In flies and mammals, transposon-directed piRNAs typically map to both strands and are produced by a “ping-pong” amplification cycle, whereby sense piRNAs direct Piwi-dependent cleavage of a primary transcript to generate the 5′ ends of antisense piRNAs and vice versa (Aravin et al., Science, 316: 744-747, 2007b; Brennecke et al., Cell, 128: 1089-1103, 2007; Gunawardane et al., Science, 315: 1587-1590, 2007; Houwing et al., Cell, 129: 69-82, 2007). Recent work suggests that Piwi-bound precursor piRNAs are trimmed by a 3′-to-5′exonuclease and then methylated on the 2′-OH of the 3′ end residue of the mature piRNA (Kawaoka et al., Mol Cell., 43: 1015-1022, 2011). In mice an abundant class of piRNAs, patchytene piRNAs, originates from large genomic clusters (Aravin et al., Nature, 442: 203-207, 2006). These piRNAs are not generated by the ping-pong cycle, but instead appear to be processed directly from a single-strand precursor by an unknown mechanism.

The C. elegans piRNAs, known as 21U-RNAs, are an abundant class of germline-expressed small RNAs that interact with the PIWI ortholog PRG-1 (Batista et al., Mol Cell., 31: 67-78, 2008; Das et al., Mol Cell., 31: 79-90, 2008; Ruby et al., Cell, 127: 1193-1207, 2006). These Piwi-interacting (pi) RNAs are a class of germline-expressed small RNAs that have been linked to epigenetic programming in metazoan. Similar to mammalian pachytene piRNAs, 21U-RNAs are diverse in sequence and the overwhelming majority lack perfectly-complementary RNA targets. The C. elegans piRNAs (21U-RNAs) are defined by more than 15,000 genomically-encoded species. Unlike mammalian piRNAs, however, 21U-RNAs do not appear to be processed from long RNA precursors. Instead, they derive from individual gene-like loci that are dispersed within two large clusters on chromosome IV (Cecere et al., Mol Cell., 47(5): 734-45, 2012; Ruby et al., Cell, 127: 1193-1207, 2006). Within these clusters, more than 15,000 distinct 21U-RNAs are expressed from both strands and reside within introns and intergenic regions, but are rarely found in coding regions (Batista et al., Mol Cell., 31: 67-78, 2008; Ruby et al., Cell, 127: 1193-1207, 2006). The presence of a conserved 8 nucleotide (nt) motif and A/T-rich region upstream of each 21URNA led Ruby et al. (Cell, 127: 1193-1207, 2006) to suggest that 21U-RNAs are independently expressed loci. Consistent with this idea, a recent study identified Forkhead-family transcription factors that associate with the 8 nt motif and whose activity was correlated with 21U-RNA expression (Cecere et al., Mol Cell., 47(5): 734-45, 2012).

The methods of the invention are particularly useful in understanding the biogenesis and expression of piRNAs as these small RNAs are suggested to play a role in gene regulation. Examples 10-18 demonstrate the use of the methods of the invention (methods that enrich the 5′ ends of Pol II transcripts) to understand the origin of C. elegans 21U-RNAs. Examples 10-18 demonstrate that a species of capped-short (cs) RNA is frequently expressed bidirectionally at Pol II loci in C. elegans. Interestingly, at annotated 21U-RNA loci, csRNAs originate precisely 2 nt upstream of the mature piRNA species, suggesting that csRNAs are piRNA precursors. In addition, it is shown that csRNAs associated with TS sites genome-wide define a second class of 21U-RNA loci, and nearly double the number of piRNA species available for genome surveillance. Examples 10-18 demonstrate that the methods of the invention have general utility in TS site identification and 5′ anchored RNA-expression profiling.

C. Identification of TS Sites

To illustrate the general utility of this approach it is demonstrated herein that CapSeq can be used to identify TS sites of genes expressed, e.g., genes in mouse testes, including candidate TS sites for primary miRNA transcripts and piRNA clusters. In order to identify TS sites (or candidate TS sites, one or a population of 5′ sequence tags generated using the methods of the invention cap be mapped to genes within a database of interest.

5′ sequence tags of the invention can be instrumental in understanding gene regulation, determination of transcriptional start sites, understanding the biogenesis of short non-coding RNAs, etc. 5′ sequence tags can be subject to bioinformational analysis and organized into databases representative of a variety of cell types, species, developmental stages, etc.).

In general, a 5′ sequence tag results from quick sequencing of a cloned cDNA. The cDNAs used for 5′ sequence tag generation are typically individual clones from a cDNA library generated using the rapid enzymatic enhancement of capped RNAs or CapSeq protocols of the invention. The resulting cDNA sequence is not necessarily a perfect copy (or complement) of the RNA species.

5′ sequence tags (or “reads”) can be mapped to specific chromosome locations using physical mapping techniques, such as radiation hybrid mapping, Happy mapping, or FISH. Alternatively, if the genome of the organism that originated the 5′ sequence tag has been sequenced, one can align the 5′ sequence tag sequence to that genome using a computer. Reads from a cDNA library can be limited, for example, by removing reads mapping to genes or RNAs not of interest. For example, reads mapping to structural RNAs can be removed from a sample or population of reads intended to be the subject of further analysis.

In this respect, 5′ sequence tag are a valuable tool to identify predicted transcription start (TS) sites throughout the genome, which leads to the prediction and/or understanding of gene transcripts, their regulation, and ultimately their function. Moreover, the situation in which the 5′ sequence tags are obtained (tissue, organ, organism, cell type, disease state, developmental state, etc.) gives information on the conditions in which the corresponding transcript is acting. Ultimately, 5′ sequence tag (or portions thereof) can be used to design probes and/or primers based on the nucleotide sequence contained therein, for example, in better understanding several important aspect of gene regulation.

Transcription start (TS) sites identified by mapping 5′ sequence tags of the invention can be considered “candidate” TS sites, and subject to further analysis, for example, bioinformational analysis, sequence analysis, comparison with data generated using other comparable or compatible sequencing approaches, etc. In exemplary embodiments of the invention, candidate TS sites can be mapped to sequences in databases including, but not limited to miRNA databases, whole genome databases, non-coding RNA databases, etc.

D. Clinical and Related Uses

The protocols of the invention are particularly useful for analyzing clinical samples, e.g., for gene expression analysis, because Capseq is rapid and uses very little starting material, which is often limiting when using clinical samples, e.g., tissue, cell, body fluid samples and the like. The methods of the invention also provide added benefit based in the fact that the methods are liquid-based. Thus the skilled artisan can process multiple samples in a parallel fashion.

The protocols of the invention are also particularly well-suited for processing samples (biological samples) in which the nucleic acid, e.g., RNA, is partially degraded. This is often the case when using, for example, certain biopsy samples or clinical samples. Using the methods of the invention, analysis of degraded RNA is possible. By contrast, other art-recognized methods are not suited for use with degraded RNA samples.

The protocols of the invention are also particularly suited for detecting alternative promoter usage or mRNA 5′ ends from cancer samples, embryonic stem cells, and the like.

In essence, the methods of the invention can replace the art-recognized CAGE methodology and can also be used as a common RNA-seq way to compare gene levels (e.g., by comparing the tag portions of reads of the invention).

Comparing to other art-recognized protocol, RNA-seq (poly A purification) is biased for poly A containing RNA while using Ribo-minus requires the use of different kits to remove rRNA for different organisms. By contrast, CapSeq can clone all mRNA and is not subject to species difference.

### III. Compositions

The invention also pertains to compositions made using the enzymatic method of enriching for capped RNAs described herein.

In one embodiment, the invention pertains to an RNA mixture comprising RNA molecules comprising transcriptional start sites, wherein the RNA molecules comprise monophosphorylated 5′ termini or hydroxyl 5′ termini, wherein the mixture is substantially free of ribosomal RNA, RNA with triphosphorylated 5′ termini, and 5′ m7G capped RNA.

Because the instant method does not involve a poly A+ selection step, in one embodiment, the mixture comprises RNAs substantially free of poly-A at the 3′ terminus.

In a further embodiment, the mixture is substantially free of RNA with poly-A at the 3′ terminus.

In one embodiment, the mixture comprises RNA with a size of less than 200 nucleotides. In one embodiment, the mixture comprises RNA with a size of between about 50 nucleotides and about 200 nucleotides. As used in this context, the term about refers to +/−5 nucleotides. For example, in one embodiment, the mixture comprises RNA with a size of between 45 and 205 nucleotides. In another embodiment, the mixture comprises RNA with a size of between about 75 and 200 nucleotides. In another embodiment, the mixture comprises RNA with a size of between about 75 and 175 nucleotides. In another embodiment, the mixture comprises RNA with a size of between about 75 and 150 nucleotides. In another embodiment, the mixture comprises RNA with a size of between about 75 and 125 nucleotides. In another embodiment, the mixture comprises RNA with a size of between about 75 and 100 nucleotides. In another embodiment, the mixture comprises RNA with a size of between about 50 and 175 nucleotides. In another embodiment, the mixture comprises RNA with a size of between about 50 and 175 nucleotides. In another embodiment, the mixture comprises RNA with a size of between about 50 and 150 nucleotides. In another embodiment, the mixture comprises RNA with a size of between about 50 and 125 nucleotides. In another embodiment, the mixture comprises RNA with a size of between about 50 and 100 nucleotides. In another embodiment, the mixture comprises RNA with a size of between about 50 and 75 nucleotides.

In one embodiment, the RNA mixture comprises a substantially pure population of RNA molecules having a size of between about 130 nucleotides and 170 nucleotides.

### IV. Kits

The present invention also provides kits or systems for performing any of the methods of the invention, including any of the steps of said methods.

For example, in one embodiment, the present invention provides a kit comprising: a) a first component comprising a 5′ monophosphate-dependent exonuclease, a second component comprising a phosphatase, and a third component comprising a decapping enzyme. In one embodiment, the kit further comprises a control sample. In one embodiment, the kit further comprises instructions for use of the kit to enrich for capped RNAs.

All publications, patents, and patent applications cited herein, whether supra or infra, are hereby incorporated by reference in their entirety. **************

The above disclosure generally describes the present disclosure, which is further exemplified by the following examples. These specific examples are described solely for purposes of illustration, and are not intended to limit the scope of this disclosure. Although specific targets, terms, and values have been employed herein, such targets, terms, and values will likewise be understood as exemplary and non-limiting to the scope of this disclosure.

### Example 1

### An Enzymatic Method for Quantitative Profiling of RNA 5′ Ends

Conventional RNAseq strategies often require large amounts of RNA (e.g., on the order of at least 5 to 10 μg of total RNA, but often much higher amounts), as well as positive or negative oligo-based selection steps. Although commercially available kits claim that 1 μg or less RNA can be used for these purposes, these amounts are on the border of the detection limit, putting into question the quality of the resulting cDNA library. Such enrichment steps often require purification columns, which can be inefficient and bias the population of enriched products. The present invention provides for a fast, efficient, and sensitive enzymatic method for eliminating contaminating RNA sequences and enriching for RNAs that contain 5′ cap structures.

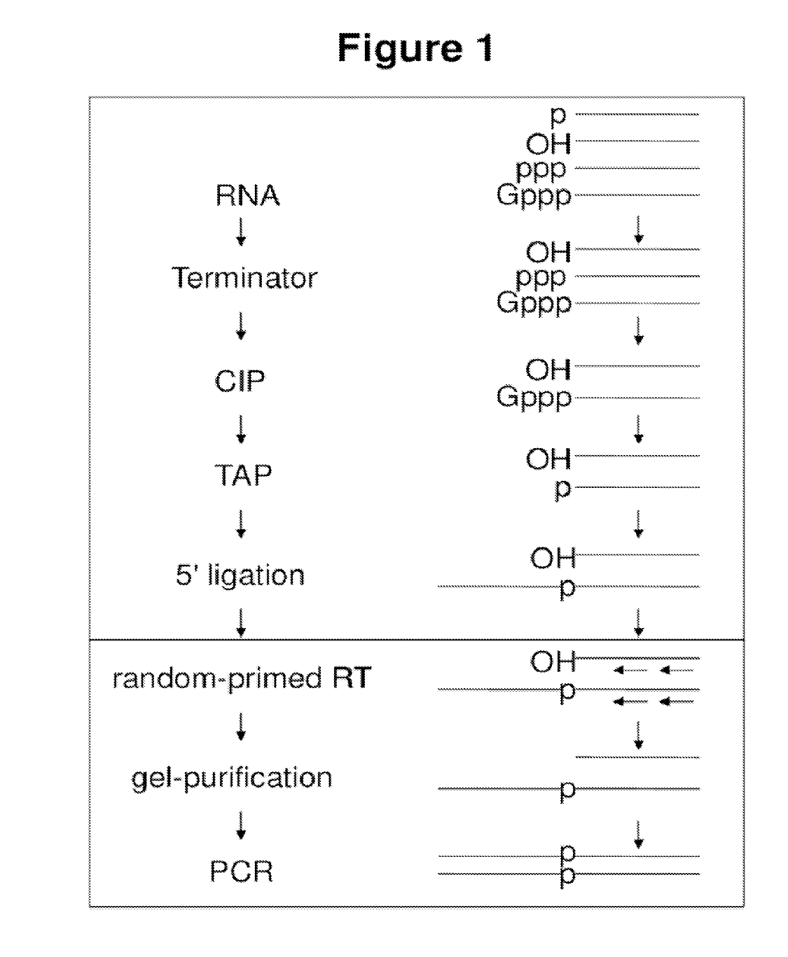

The method, in brief, entails purification of total RNA from a target cell population. As shown in FIG. 1, the total RNA population is a mixture of various RNA species, i.e., those with 5′ ends characterized by monophosphate (e.g., rRNAs), —OH (e.g., degraded RNA), triphosphate (e.g., pre-tRNA), and capped triphosphates. The first step involves removing contaminating rRNA species using the a 5′-phosphate-dependent exonuclease (e.g., “Terminator”; Epicentre, Madison, Wis.). The second step involves using a phosphatase (e.g., CIP; NEB) to remove triphosphates of RNA species characterized by triphosphate 5′ ends, thereby rendering these RNA species unamenable to cloning. The third step involves processing 5′ capped RNAs with a decapping enzyme (e.g., tobacco acid pyrophosphatase (TAP), an enzyme that hydrolyzes the phosphoric acid anhydride bonds in the triphosphate bridge of the cap structure). This releases the cap nucleoside and generates a 5′-monophospohorylated terminus, allowing for selective and effective enrichment and cloning of 5′ capped RNAs. The monophosphate end-containing RNA species can then be ligated to a linker (e.g., a Solexa or other linker) using RNA ligase in a 5′ ligation reaction. Following linker ligation, random primers can be used to reverse transcribe the 5′ monophosphate end-linker containing RNA species and 5′-OH containing RNA species. This may be followed by gel purification for size selection, and PCR using linker-specific primers to enrich for cDNA derived from capped RNA.

### Example 2

### Materials and Methods

### Chemicals, Enzymes, and Oligos

Terminator (Epicentre; TER51020), CIP (NEB; MO290S), T4 RNA ligase (Takara 2050A), SUPERaseIn™ (Ambion; AM2696), TAP (Epicentre; T19050), DNase I (Ambion; AM2222), Superscript III (Invitrogen; 18080, with 10× buffer without Mg²⁺), ExTaq (Takara; RR001B), Taq (Roche), pCR 2.1 TOPO (Invitrogen; K4500-02), RNase A (Ambion; AM2270), RNase H (Ambion; AM2292), Phase lock gel heavy (5PRIME), spin-x 0.45 μm (Costar; 19442-758), 10 bp DNA marker (Invitrogen 10821-015), phenol/chloroform pH 6-8 (for DNA, pH 7-8 is better), glycoblue (Ambion; AM9515), glycogen (Ambion; AM9510). With respect to oligos, the reverse transcription oligo (RT oligo) was 5′-CAGAAGACGGCATACGANNNNNNNN-3′, the solexa 3 oligo was 5′-CAAGCAGAAGACGGCATACGA, the CMo13279 oligo was 5′-GTTCTACAGTCCGACGATC-3′, the CMo13278 oligo was 5′-AATGATACGGCGACCACCGACAGGTTCAGAGTTCTACAGTCCGACGATC-3′, the M13 forward primer was 5′-GTAAAACGACGGCCAG-3′, the M13 reverse primer was 5′-CAGGAAACAGCTATGAC-3′, and the T7 primer was 5′-TAATACGACTCACTATAGGG-3′.

The 5′ linker was a DNA/RNA hybrid oligo (RNA underscored, otherwise DNA):

TCTACrArGrUrCrCrGrArCrGrArUrC-barcode
barcodeA-rTrGrArC
barcodeB-rCrArGrT
barcodeC-rGrCrTrG
barcodeD-rArTrCrA

### Precipitation

Precipitation involved adding 1/10^{th} volume of sodium acetate and 20 μg glycogen or 30 μg glycoblue. To this mixture was added 1-1.5× isopropanol or 3-4× ethanol. The resulting mixture was chilled at −20° C. for 30 min and centrifuged @ 20,000 g for 15 min at 4° C.

### Phase Lock

Phase lock involved centrifuging the sample at 13,000×g for 2 minutes. This was followed by mixing phenol/chloroform with equal volume of RNA/DNA (at least 100 μL each). The mixture was mixed by finger tapping, followed by centrifugation at 13,000×g for 4 minutes. The salt concentration was no more than 0.5M.

### Gel Elution

Gels were made with Biorad cassette 345-9902 and the Biorad criterion system was used to run the PAGE gels. Gels were cut to be in small pieces, and cracked in 1.5 mL siliconized tubes by grinding the gel against the wall. RNA/DNA elution buffer (0.8 mL; 10 mM Tris-Cl pH 7.5, 1 mM EDTA, and 0.3M NaCl) was added to elute overnight. The spin-x column was used to remove gel. The resulting product was precipitated as described above with isopropanol without additional salt.

### Example 3

### Detailed Procedure

### RNA Isolation

RNA can be isolated from desired tissue or whole organisms (e.g., C. elegans) using art-recognized techniques or commercially available kits (e.g., Qiagen).

### Terminator Treatment

Terminator™ 5′-phosphate-dependent exonuclease (Epicentre, Madison, Wis.) treatment involved setting up a 20 μL reaction with the following components:

| | |
|---|---|
| Buffer A | 2 μL (stock 10X) |
| SuperaseIN | 1 μL (stock 20U/μL) |
| BSA | 1 μL (stock 1 μg/μL) |
| RNA | final concentration of 0.1 μg/μL |
| Terminator | 2 μL (stock 1U/μL) |
| Water | to 20 μL |

This mixture was incubated at 30° C. for 2 hours.

### CIP Treatment

The following components were added to the Terminator treatment mixture from above.

| | |
|---|---|
| DNase I buffer | 10 μL (stock 10X) |
| SuperaseIN | 1 μL (stock 20U/μL) |
| DNase I | 2 μL (2U/μL, Ambion) |
| CIP | 2 μL (10U/μL, NEB) |
| Water | 65 μL |

This mixture was incubated at 37° C. for 30 minutes, followed by phenol/chloroform/glycoblue precipitation using art-recognized techniques.

### TAP Treatment

The following components were added to the precipitated mixture from the CIP treatment above.

| | |
|---|---|
| TAP buffer | 1 μL (stock 10X) |
| SuperaseIN | 0.5 μL (stock 20U/μL) |
| TAP enzyme | 0.25 μL (stock 10U/μL) |
| Water | 8.25 μL |

This mixture was incubated at 37° C. for 1 hour, followed by phenol/chloroform extraction without glycoblue (since glycoblue was added in the previous step).

### 5′ Ligation

The following components were added to the pellet obtained from the precipitate after TAP treatment:

| | |
|---|---|
| Buffer (w/ATP) | 1 μL (stock 10X) |
| SuperaseIN | 0.5 μL (20U/μL) |
| BSA | 1 μL (stock 1 μg/μL) |
| T4 RNA ligase | 0.5 μL (stock 40U/μL) |
| 5′ linker | 0.25 μL (stock 200 μM) |
| DMSO | 1 μL (stock 100%) |
| Water | 5.75 μL |

This mixture was incubated at 15° C. for 6 hours, followed by 4° C. overnight. The next day, the mixture was subjected to phenol/chloroform extraction without glycoblue.

The 5′ linker barcodes were as follows: embryonic (barcodeA), L1 developmental stage (barcodeB), L3 developmental stage (barcodeC), and YA developmental stage (barcodeD).

### Reverse Transcription

The following components were added to the pellet obtained from the precipitate after the 5′ ligation step:

| | |
|---|---|
| RNA | pellet |
| RT oligo | 1 μL (stock 50 μM) |
| dNTP | 1 μL (stock 10 mM) |
| Water | to 11 μL |

This mixture was incubated at 65° C. for 5 minutes, followed by incubation on ice for 2 minutes. The following was added to this mixture:

| | |
|---|---|
| RT buffer | 4 μL (stock 5X) |
| DTT | 2 μL (stock 100 mM) |
| SuperaseIN | 0.5 μL (20U/μL) |
| SuperScriptIII | 0.5 μL (stock 200U/μL) |

This mixture was incubated at 15° C. for 10 minutes, 25° C. for 10 minutes, 50° C. for 50 minutes, and then 85° C. for 5 minutes. Following this, 1 μL of RNase A and 1 μL of RNase H were added to the mixture, which was subsequently incubated at 37° C. for 20 minutes.

Linear PCR to Amplify cDNA

The following components were added to the mixture from the RT reaction above:

| | |
|---|---|
| PCR buffer | 10 μL (stock 10X) |
| 13279 primer | 0.5 μL (stock 10 μM) |
| dNTPs | 8 μL (stock 2.5 mM) |
| ExTaq | 0.5 μL (stock 2.5U/μL) |
| Water | to 100 μL |

The PCR conditions were as follows:

One cycle: 94° C. for 60 seconds

12 cycles: 94° C. for 20 seconds
52° C. for 20 seconds
72° C. for 30 seconds

This mixture was phenol/chloroform extracted without addition of glycoblue. This was followed by 15% PAGE (with 7M urea) gel purification to obtain PCR products of 130-170 nucleotides (total RNA containing 5S and 5.8S rRNAs were used as markers).

### Testing PCR

Purified linear PCR products from the step above were used for a test PCR reaction. This step, although optional, can be carried out to identify the optimal PCR cycles for amplification. Such optimization greatly increases the quality of the cDNA library. PCR products at different cycles are sampled to determine when enough product is obtained without over-cycling.

First, gel purified PCR products were precipitated with glycoblue and resuspended in 20 μL of water. The test PCR reaction was set up as follows:

| | |
|---|---|
| PCR buffer | 5 μL(stock 10X) |
| CMo13279 primer | 0.5 μL (stock 10 μM) |
| Solexa 3 primer | 0.5 μL (stock 10 μM) |
| cDNA | 5 μL |
| dNTPs | 5 μL (stock 2.5 mM) |
| ExTaq | 0.5 μL (stock 2.5U/μL) |
| Water | 34 μL |

The PCR conditions were as follows:

One cycle: 94° C. for 20 seconds

15 cycles: 94° C. for 20 seconds
50° C. for 20 seconds
72° C. for 30 seconds
   Following this PCR reaction, 5 μL of 10 μM solexa 3 primer and 5 μL of 10 μM CMo13278 primer were added. CMo13278 contains the full-size sequence (total of about 50 nucleotides) required for Solexa sequencing, while CMo13279 only contains a portion of the sequence, thus reducing primer dimers. PCR products were sampled at 18, 21, 24, and 27 cycles, followed by running on an 8% gel to determine the optimal cycle number.

### Production PCR and Gel Purification

The above PCR reaction was carried out in two tubes for each sample, followed by purification of PCR products using 8% PAGE without urea. A 10 bp marker was used to locate the position of PCR products of the correct size range (i.e., 130-170 nucleotides).

### Quantification, Dilution, TA Cloning, and Transformation

Samples from the gel purification above were resuspended in 15 μL Tris-Cl (pH 7.5) one at a time to avoid denaturing, as DNA denatures when totally dried. The resuspended samples were subjected to TA cloning as follows:

| | |
|---|---|
| PCR product | 2 μL |
| TA cloning buffer | 0.45 μL (stock 10X) |
| Taq polymerase | 0.1 μL |
| dNTPs | 0.45 μL (stock 2.5 mM) |
| Water | 1.5 μL |

This mixture was incubated at 72° C. for 15 minutes. Following this, 1 μL of salt buffer and 0.5 μL of enzyme was added. This mixture was incubated at room temperature for 30 minutes, followed by transformation with TOP10 competent cells. Transformed cells were streaked onto ampicillin and β-galactosidase containing plates.

### Colony PCR, PCR Purification, and Manual Sequencing

Colonies of transformed bacteria on the streaked plates were subjected to colony PCR as follows:

| | |
|---|---|
| PCR buffer | 2 μL (stock 10X) |
| M13F primer | 0.4 μL (stock 10 μM) |
| M13R primer | 0.4 μL (stock 10 μM) |
| Colony | |
| dNTPs | 0.2 μL (stock 25 mM) |
| Taq polymerase | 0.2 μL (stock 5U/μL) |

PCR conditions were as follows:

One cycle: 94° C. for 120 seconds

15 cycles: 94° C. for 20 seconds
50° C. for 20 seconds
72° C. for 30 seconds
   PCR was followed by PCR purification and econo sequencing. The econo sequencing mix was as follows: 1 μL of T7 primer (1 μM stock), 3 μL of template, and 6 μL of water, and was carried out at the CFAR facility at University of Massachusetts Medical School.

### Solexa Sequencing

Alternative to manual sequencing, the purified PCR products were subjected to Solexa sequencing (Illumina) using Solexa sequencing tags in accordance with the manufacturer's instructions. The sequence obtained was 75 nucleotides long from single end sequencing.

### Example 4

### Comparison of Annotated mRNA Transcripts with Sequencing Reads

RNA was extracted from C. elegans from the following developmental stages: embryonic, L1, L3, and young adult (YA). RNA from each of these stages were processed using the method as described in Examples 1-3, followed by deep sequencing with Illumina Solexa sequencing technology.

C. elegans were grown at 20° C. on E. coli strain OP50 as a food source and harvested at different developmental stages, as indicated. Worms were then dounced with phenol (pH6.7-7) with a metal douncer to expose RNA. The water phase of the extract was precipitated with 0.3M sodium acetate (NaAc) pH 5.2 and 1 volume of isopropanol at −20° C. for at least 20 minutes. The RNA was precipitated by centrifugation at 20,000×g for 15 minutes at 4° C., washed once with 70% cold ethanol, and dissolved in water. To clean the RNA further, another equal volume phenol extraction was carried out as described above.

Genome information (WS215) was downloaded from the publicly available WormBase database. The mapping was performed using bowtie in which only 1 mismatch at most for each 20 nt was allowed. That is, e.g., if the read is 72 nucleotides long, the number of mismatches cannot exceed 3. Before matching, the splice leader (SL) was searched for each read, and removed if present, using a custom PERL script. Given barcoding issues (i.e., a certain percentage of barcodes are shorter than expected) and issues related to removing mutated SL sequences (some SL's are larger than others), the resulting reads could have 3′ ends off by 1-2nt than expected. Accordingly, matching reads beginning at the same position on the same genome strand are combined and the end of the combined reads is represented by the end of the longest reads among them using a custom PERL script. Each sample was normalized to 5 million mRNA sequences and visualized using a generic genome browser.

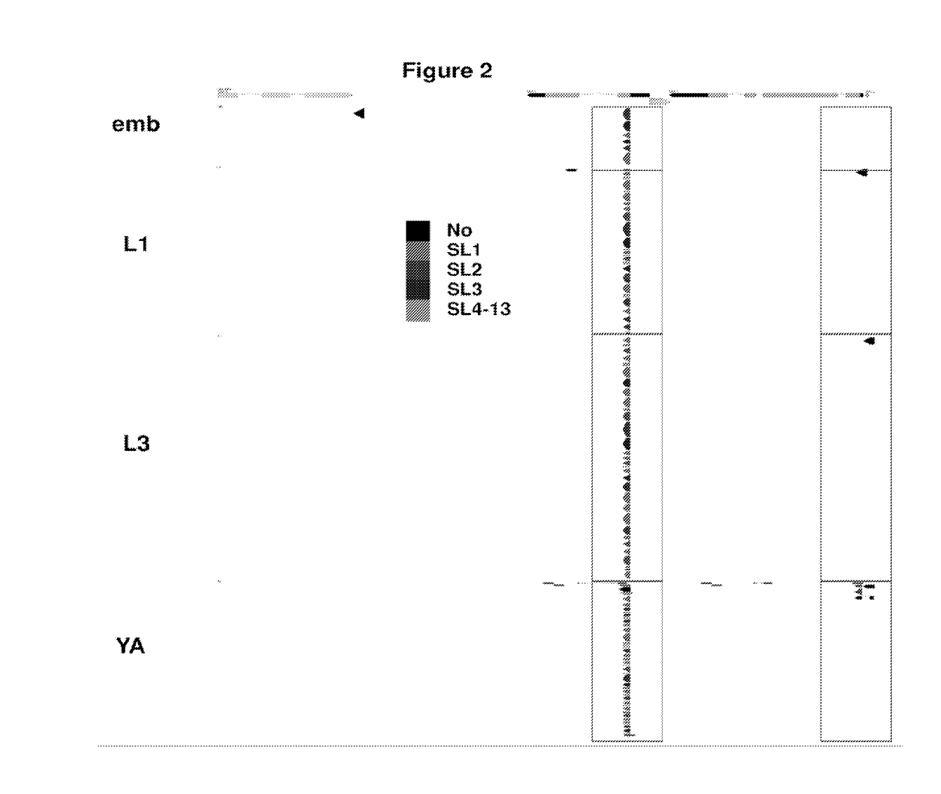

FIG. 2 displays the 5′ end of transcripts obtained by the method set forth in the Examples and Solexa sequencing, and overlays the sequencing reads onto the genomic structure of four annotated transcripts (T24H10.5, T24H10.6, RNAz-513084, and T24H10.3). Each arrow in the Figure represents the determined transcriptional start site. The height of an arrow represents the number of reads on a log scale. The black arrow indicates that no trans-splicing has occurred. Colored arrows represent trans-splicing events, in which an SL leader sequence is spliced with the 5′ end of a transcript, thereby producing the mature 5′ end of a transcript. The sequence reads in FIG. 2 suggest that the T24H10.6 gene transcript is annotated incorrectly, i.e., the arrows representing sequence reads do not begin from the 5′ most end of the annotated transcript.

### Example 5

### Comparison of Annotated Drh-3 Transcripts with Sequencing Reads

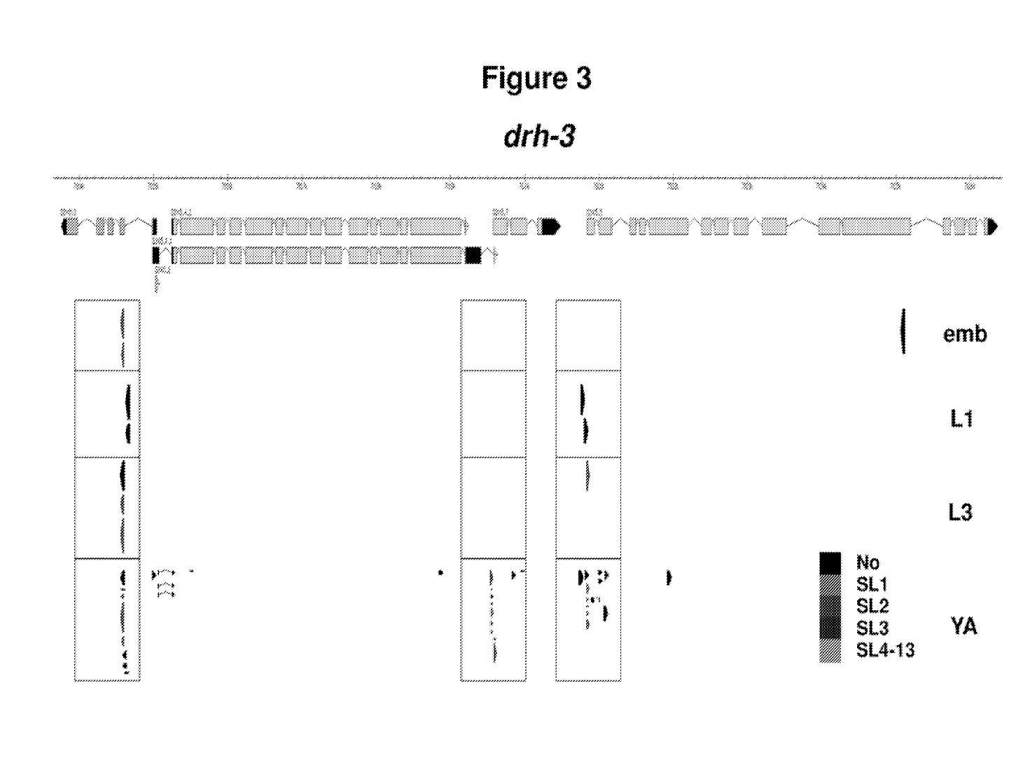

Using the procedure described in Example 4, the 5′ ends of drh-3 transcripts obtained were compared with annotated drh-3 (D2005.5) transcripts. drh-3 encodes a RNA helicase-like protein which shares similarity to dicer-related helicases. As shown in FIG. 3, this particular genomic locus has 6 distinct transcripts, including 5 mRNA transcripts. As mentioned in Example 4, each arrow represents the transcriptional start site, and the height of the arrows represents the number of reads on a log scale. The black arrow indicates that no trans-splicing has occurred, while colored arrows represent trans-splicing events. As can be appreciated from FIG. 3, gene D2005.3 has a major isoform (not annotated in the C. elegans genome browser) whose 5′ end detected by the instant method resides at or just upstream of the second predicted exon. Indeed there is no experimental evidence in the instant method data set for the predicted longer isoform suggesting that this locus is incorrectly annotated. With respect to the remaining 5 genes, sequence reads from this data set provide experimental evidence that several of the annotations for these genes are correct, i.e., arrows are located at the predicted 5′ end of the annotated sequences (see, e.g., D2005.5). The data set detects alternative (non-annotated) 5′ ends for D2005.7, several of these are transpliced to SL2,3, n, a hallmark of co-transcriptional processing as part of a polycistronic transcript that may include D2005.4, as well as a slightly off-set SL1 transpliced isoform that may reflect transcription from its own promoter. The shortest annotation, D2005.8 (a 21U-RNA gene; 21ur-12756), is a small RNA whose mature form lacks a cap. Accordingly, the reads surrounding it lack the same 5′ end.

### Example 6

### Comparison of Annotated csr-1, pgl-1, and msp-76 Transcripts with Sequence Reads

Using the procedure described in Examples 4 and 5, the 5′ ends of csr-1, pgl-1, and msp-76 transcripts obtained were compared with the annotated csr-1 and pgl-1 transcripts.

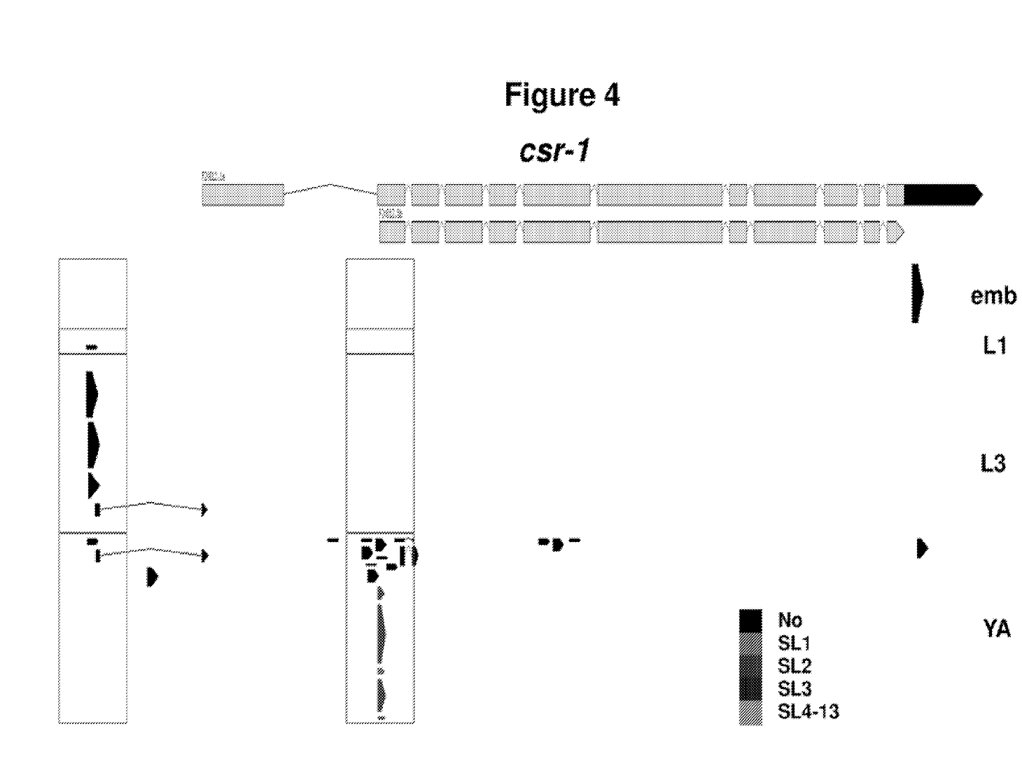

As shown in FIG. 4, csr-1 has two different transcripts, a long form (F20D12.1a) and short form (F20D12.1b). The sequence reads demonstrate that the short form is predominant in the YA developmental stage, and absent in the embryonic, L1, and L3 developmental stages. In addition the longer isoform appears likely to initiate further upstream from the annotated end. Potential internal start sites (may also represent degradation) are seen in exon 6 and in the 3′ UTR. Further experimental validation can readily determine if these potential start sites are truly capped RNAs from this locus.

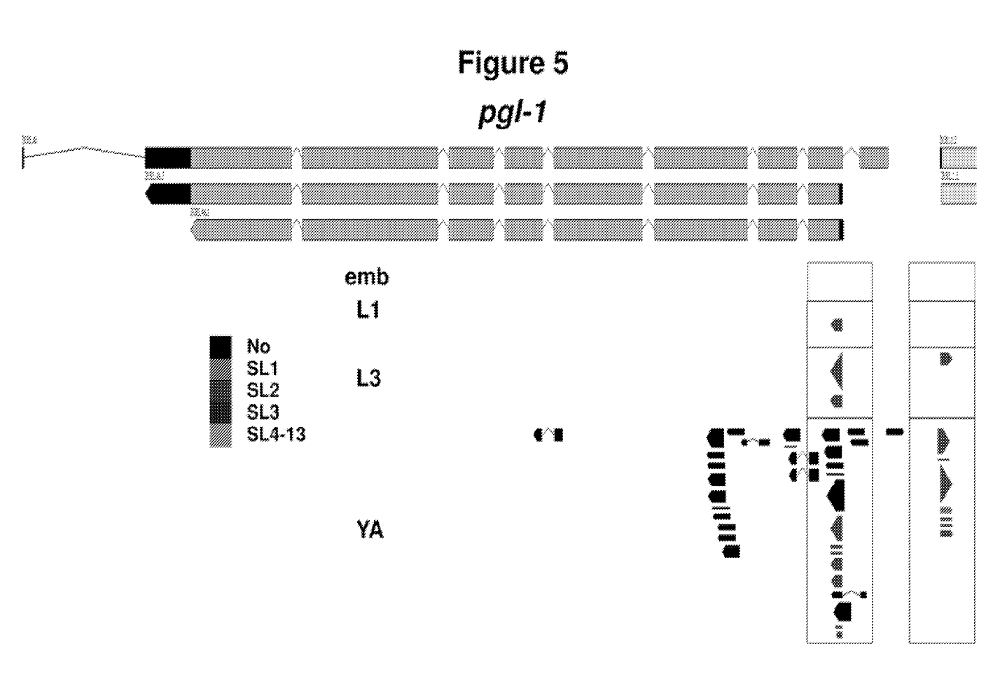

FIG. 5 shows annotation data of two different genes. Sequence reads demonstrate that the short form of pgl-1 (i.e., ZX381.4a.1 and/or ZX381.4a.2) is predominant over the longer form (ZX381.4b). With respect to the other gene (i.e., ZX381.1.1 and ZX381.1.2), both predicted transcripts are experimentally validated by the instant method.

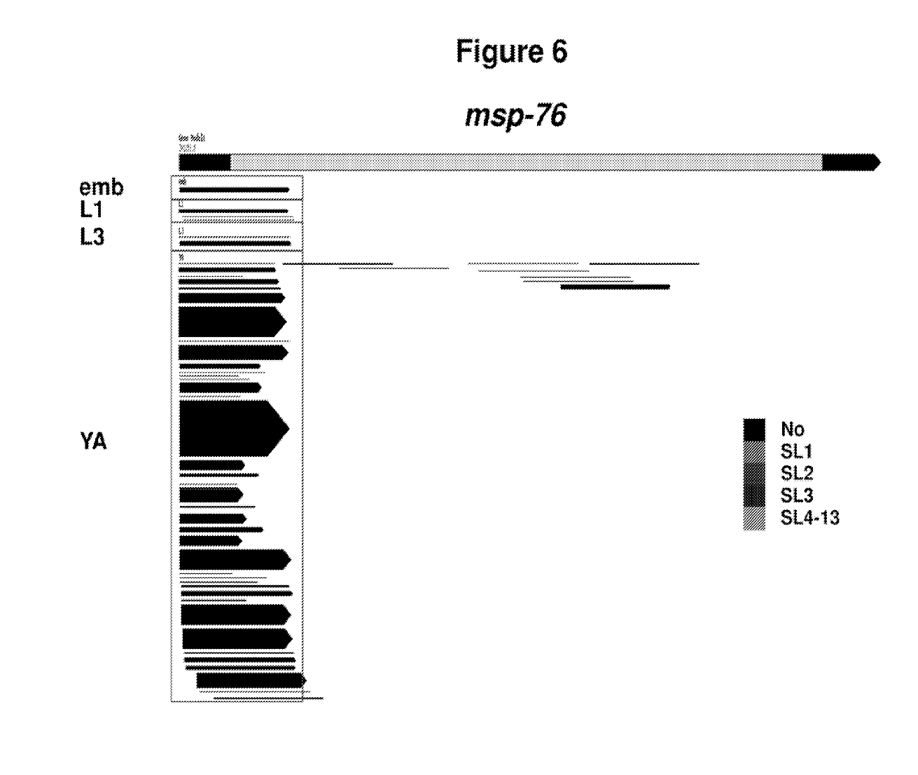

FIG. 6 shows annotation data for msp-76, a major sperm gene. Sequence reads demonstrate that msp-76 is highly expressed in the YA developmental stage, when sperm is present. This strongly supports the notion that sequencing reads can provide information on developmental expression of genes. The black bars that overlap with the intragenic region of msp-76 have read numbers that are about 1000 fold less than arrows at the 5′ end, and thus may have resulted from RNA degradation.

### Example 7

### Comparison of Annotated Small RNA Transcripts with Sequence Reads

Using the procedure described in Examples 4 and 5, the 5′ ends of transcripts for the small RNA genes K02E2.6 (i.e., K02E2.6.1 and K02E2.6.2) and F55C9.3 obtained were compared with the respective annotated sequences.

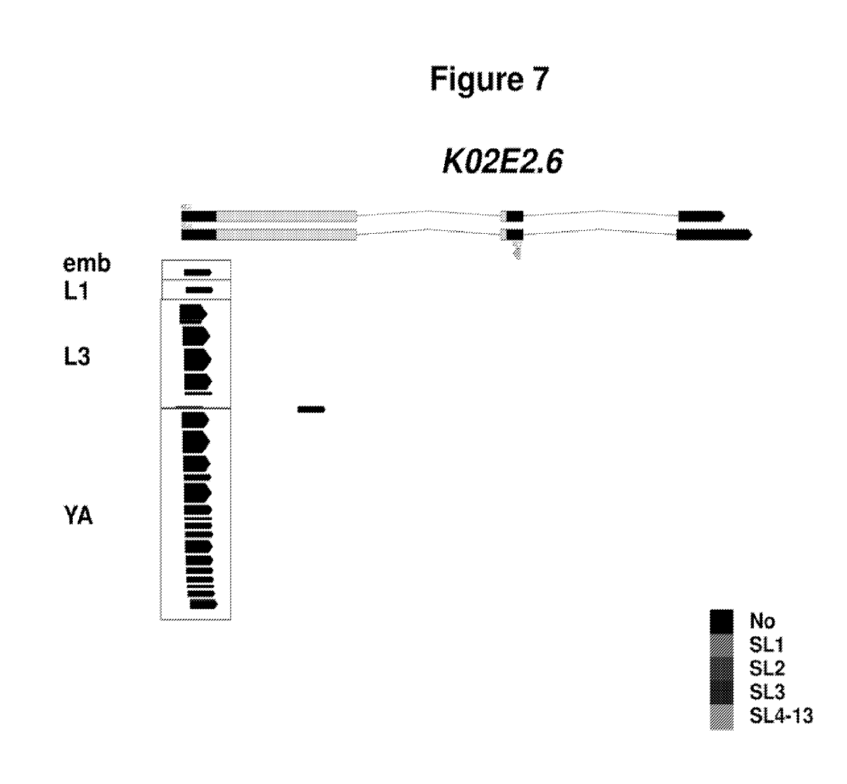

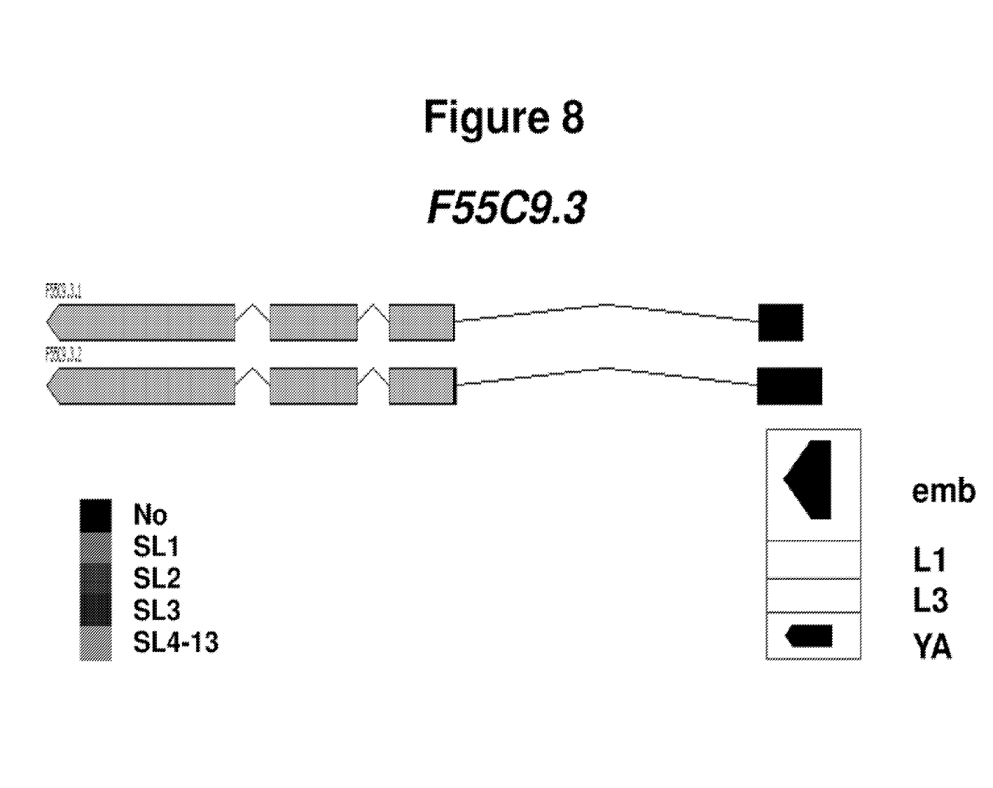

FIG. 7 shows annotation data for two transcripts of the K02E2.6 gene (i.e., K02E2.6.1 and K02E2.6.2). Sequence reads demonstrate that the annotation for the 5′ ends of these two transcripts is likely to be correct. FIG. 8 shows annotation data for two transcripts of the F55C9.3 gene (i.e., F55C9.3.1 and F55C9.3.2). Sequence reads demonstrate that of the two transcripts, the longer form is predominant, and highly expressed during the embryonic stage.

### Example 8

### Comparison of the Annotated pri-lin-4 Transcript and pri-mir-42-43-44-45 and pri-mir-229-64-65-66 Cluster Sequences with Sequence Reads

Using the procedure described in Examples 4 and 5, the 5′ ends of transcripts for pri-lin-4 and the pri-mir-42-43-44-45 and pri-mir-229-64-65-66 clusters obtained were compared with the respective annotated sequences.

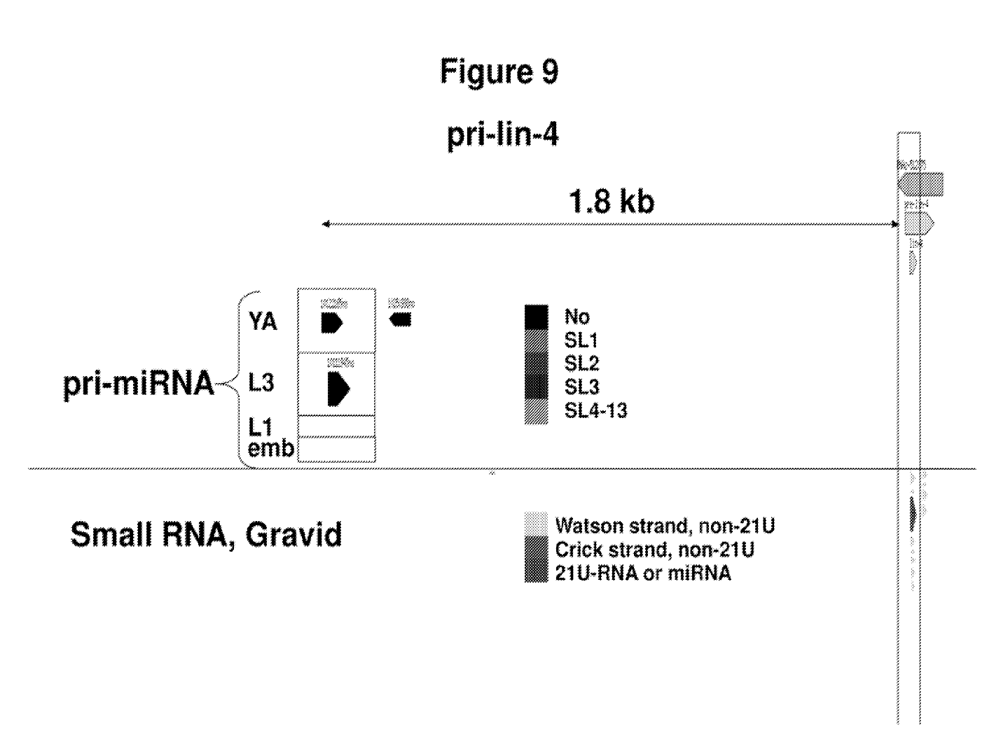

FIG. 9 shows annotation data for the transcripts of precursor (pre)-lin-4 miRNA and primary (pri)-lin-4 miRNA. Although there was no preexisting annotation information for the pri-lin-4 transcript, sequencing reads revealed the presence of a transcript with a 5′ end approximately 1.8 kb upstream of pre-lin-4 transcripts. Sequencing reads corresponding to the mature lin-4 miRNA transcripts are found in the gravid developmental stage. These data suggest that the instant methods can effectively clone the 5′ ends of as of yet unannotated transcripts (e.g., pri-lin-4). Whether the newly identified 5′ ends actually correspond to transcripts of the gene being studied (in this case, pri-lin-4) can be assessed with art-recognized techniques, e.g., Northern blot analysis to confirm the size of the transcript. In the event of low expression of the transcript, another option is RT-PCR, followed by nested PCR with gene-specific primers. The latter is the routine process of 5′ RACE or 3′RACE followed by normal sequencing to obtain the ends of a gene.

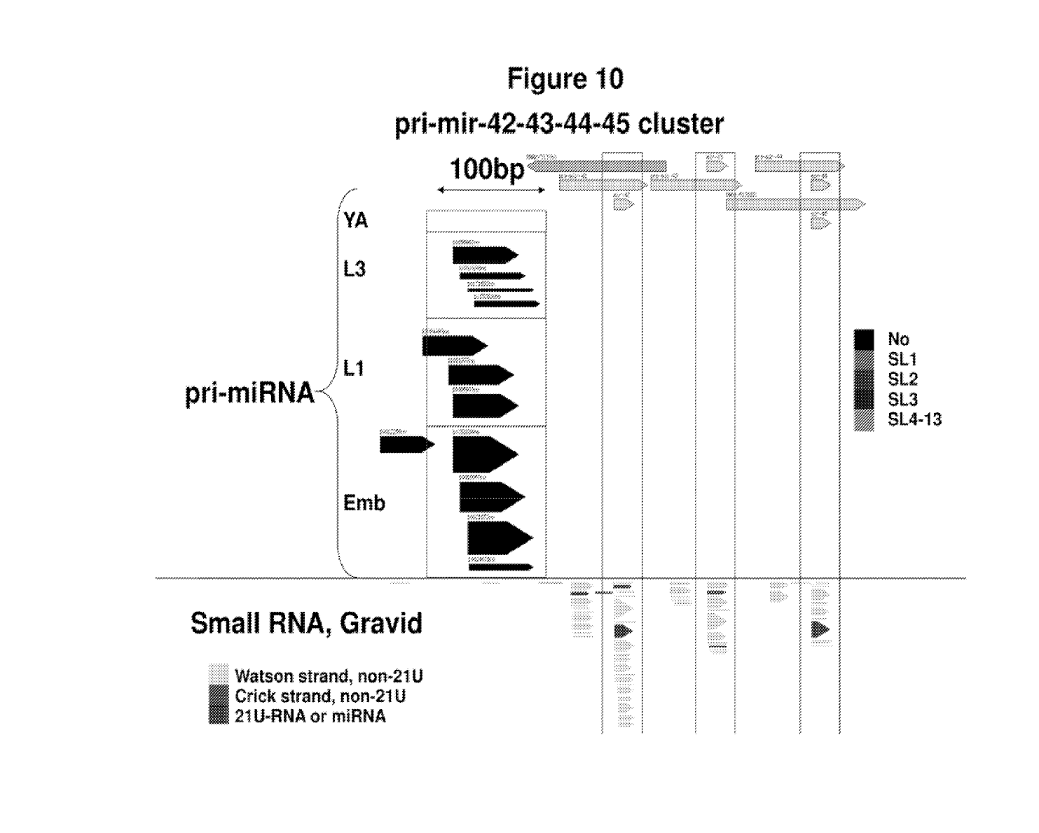

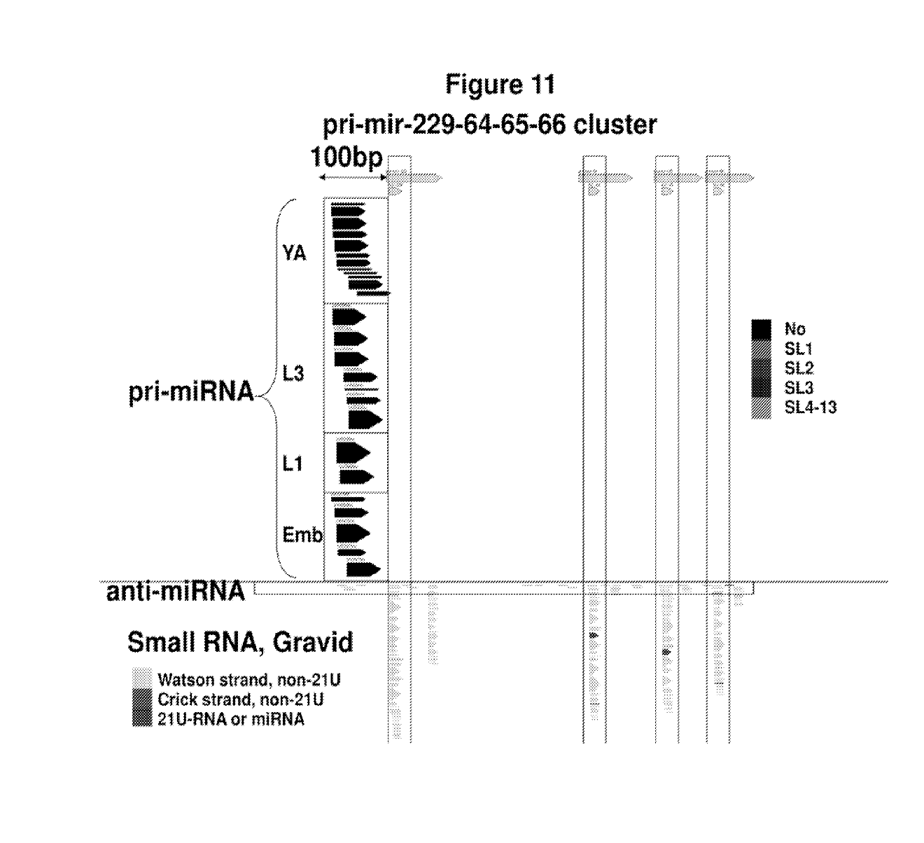

Similar results were seen for the pri-mir-42-43-44-45 and pri-mir-229-64-65-66 clusters (FIGS. 10 and 11, respectively). In these two pri-mir clusters, putative novel 5′ ends of the respectively pri-mirs are revealed by sequencing reads. These 5′ ends lie approximately 1 kb upstream of the pre-mir-42 transcript for the pri-mir-42-43-44-45 cluster, and approximately 100 bp upstream of the pre-mir-229 transcript for the pri-mir-229-64-65-66 cluster.

Besides these examples, the instant method detected potential pri-mRNAs for most of the well annotated miRNAs in C. elegans.

### Example 9

### Comparison of Annotated pri/pre-21ur-3338 Transcripts with Sequence Reads

Using the procedure described in Examples 4 and 5, the 5′ ends of transcripts for pri/pre-21 ur-3338 obtained were compared with the respective annotated sequences.

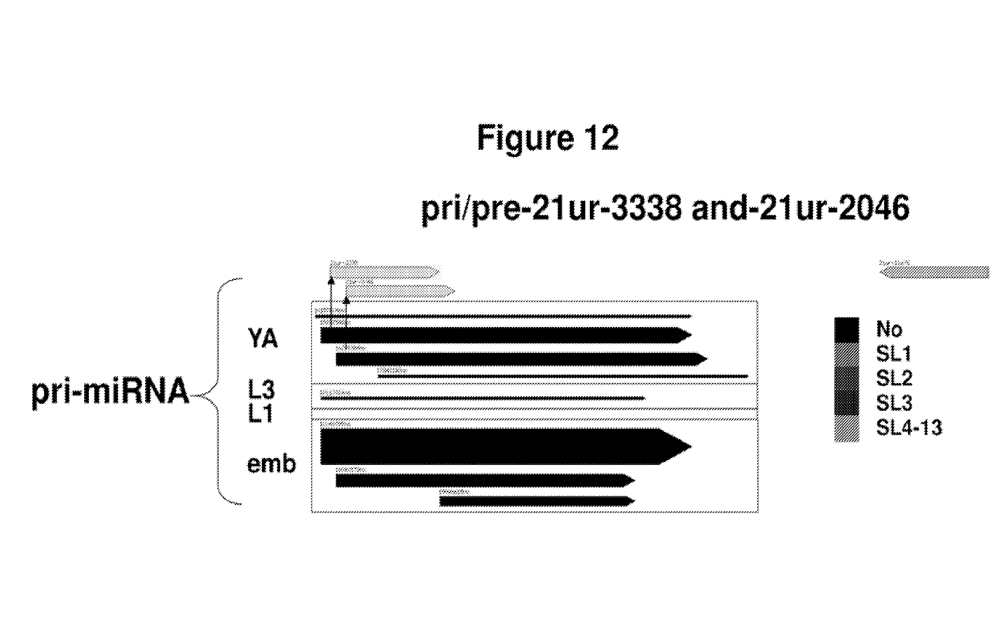

FIG. 12 shows that two major long precursor sequences were obtained the 5′ end of each precursor is aligned 2 nucleotides upstream of the annotated mature 21 ur-3338 and 21ur-2046 transcripts, respectively.

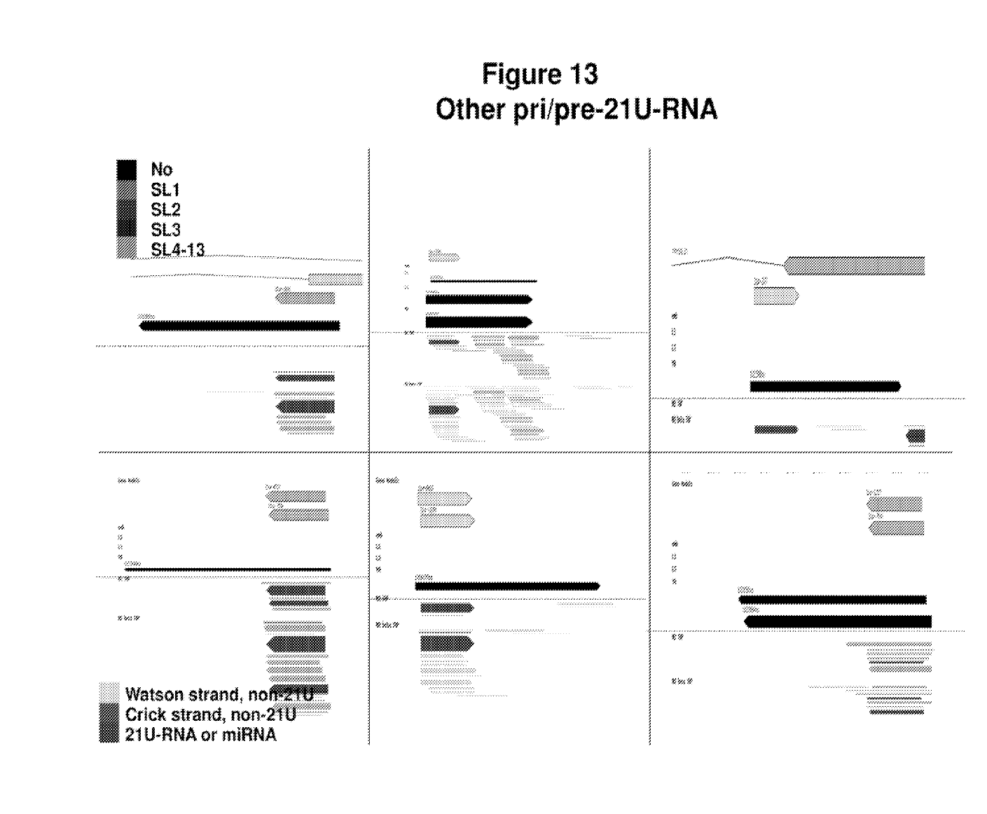

FIG. 13 shows another six cases. In about 80 of 90 cases in which a precursor is detected, the long precursors always align 2 nucleotides upstream of a known annotated 21U-RNA gene.

### Examples 10-18

In order to further explore the expression and biogenesis of 21U-RNAs, the CapSeq protocol was used to enrich 5′sequence tags of approximately 70 to 90 nucleotides in length for RNA Polymerase II (Pol II) transcripts. In addition, a previously-described enzymatic treatment (referred to here as CIP-TAP cloning; Affymetrix/Cold Spring Harbor Laboratory ENCODE Transcriptome Project, 2009) was used to enrich for capped-small (cs) RNAs like those previously found associated with promoter regions of Pol II genes in a variety of organisms (Haussecker et al., Nat Struct Mol Biol, 15: 714-721, 2008; Nechaev et al., Science, 327: 335-338, 2010; Seila et al., Cell Cycle, 8: 2557-2564, 2009; Taft et al., Cell Cycle, 8: 2332-2338, 2009). Together the data in the following Examples define candidate TS sites throughout the genome, including TS sites for rare transcripts such as primary-miRNAs (pri-miRNAs) and for pre-mRNAs associated with thousands of transpliced mRNAs. CapSeq reads usually mapped sense relative to annotated genes and often appeared to coincide with the 5′ends of the corresponding mature Pol II products. In contrast, csRNAs were frequently bidirectional upstream of protein-coding genes, with antisense-oriented csRNAs positioned ˜150 bps upstream of the sense-oriented csRNAs.

### Example 10

### Fidelity of the CapSeq Protocol

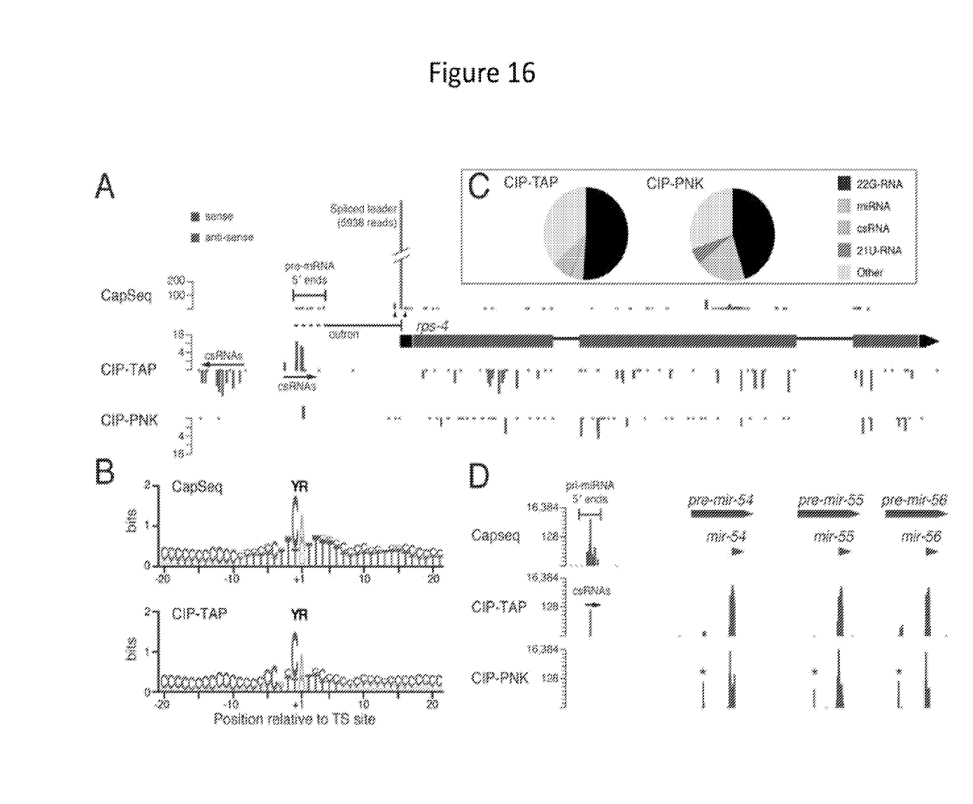

Using the CapSeq approach, we generated 5 libraries from 3 different developmental stages (L1, L3, and adult) and obtained ˜61 million reads that mapped to the C. elegans genome, including 46 million that mapped to non-structural RNAs. Visual inspection using the genome browser software “Gbrowse” revealed that most CapSeq reads are indeed enriched at the 5′ ends of genes transcribed by RNA Pol II (FIG. 16A, CapSeq panel).

To estimate the fidelity with which CapSeq defines the actual 5′ ends of transcripts, as opposed to internally truncated RNAs, the degradation rate of the spliced leader SL1, a 22 nt 5′ leader sequence trans-spliced to many C. elegans genes, was analyzed (Allen et al., Genome Res, 21: 255-264, 2011). For this analysis, five highly-expressed SL1 trans-spliced genes were chosen: alh-8, rps-24, rps-3, rps-29 and rps-15. Complete or partial SL1 reads appended with sequences that perfectly matched to the first 30 nt of each gene were analyzed. For technical and computational reasons, considered sequences with at least the last 3 nts of SL1 were considered, and sequences missing only the first nt of SL1 were excluded (see Experimental Procedures following Example 18). It was found that the average frequency of cloning a 5′-truncated product at each position (nt 3-20, relative to the last nt) of SL1 was approximately 1 in 15,000 per nucleotide relative to intact SL1 reads.

As an additional and more general approach to place an upper limit on the frequency of cloning degradation products by CapSeq, reads were considered that map to all 6,967 trans-spliced transcripts both annotated by WormBase and confirmed by the above data set. Next were compared the number of non-SL reads that start at each position of the transcript to the total number of SL-containing reads. This analysis revealed that, for every SL read detected, a non-SL read (potential degradation product) occurred at an average rate of 1 in 17,000 at each position along a transcript. Together, these data indicate that CapSeq strongly enriches for cloning the 5′-most nt of capped transcripts.

### Example 11

### Identification of New Trans-Splice Sites

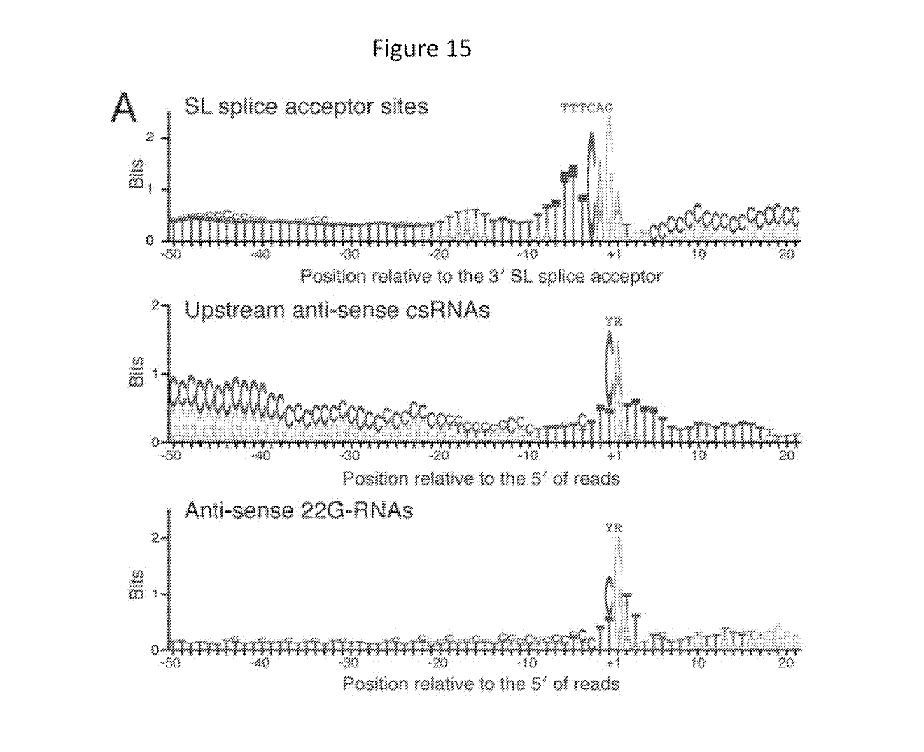

A total of 16,784 unique SL trans-splice sites (FIG. 15) were identified in the genome, including 11,073 (70%) of the 15,759 SL sites annotated in the WS215 genome.

Analysis of C. elegans CapSeq and CIP-TAP data, containing lists of trans-splice sites, transcription start sites, sense csRNAs derived from protein coding genes, and antisense csRNAs derived from protein-coding genes was conducted. Also analyzed were lists of the transcription start sites for pri-miRNAs.

Among 5,711 new trans-splice sites, 4,186 were associated with protein coding genes and mapped to splice acceptor sites within 500 nt upstream of annotated 5′ ends of transcripts (31%), to annotated exon-exon cis-splice-acceptor sites (37%), and to previously non-annotated splice-acceptor sites within annotated exons (25%) or annotated introns (6%). In addition to identifying new trans-splice sites, the analysis also revealed the relative abundance of alternative SL usage for each locus (Table 1A). For example, it was found that 25% of all unique trans-splice sites identified in this study use multiple spliced leaders.

### Example 12

### Identification of RNA Pol II TS Sites

The transcription start (TS) sites for C. elegans genes are poorly mapped. Most gene annotations in WormBase simply indicate the 5′ end of the trans-spliced exon, or the position of the AUG codon. The 5′ ends of many non-SL CapSeq reads mapped near, but did not coincide precisely with, the 5′ ends of annotated transcripts (FIG. 16A). It was hypothesized that the 5′ ends of these reads could represent TS sites. Consistent with this idea, it was noticed that these reads exhibit a strong bias for a 2 nt motif of pyrimidine (Y) purine (R) or “YR”, in which R represents the first nt (+1) in the CapSeq read and Y represents the adjacent 5′ nt (FIG. 16B). In addition, the YR motif is part of an extended consensus yYRyyy (lower case indicates weaker preference), with a strong preference for A as the R at position +1 and a slight preference for T at positions flanking the YR. This YR motif resembles the initiator element required for RNA Pol II transcription initiation in mammals, plants and flies (de Hoon et al., Biotechniques 44: 627-628, 630, 632, 2008; Juven-Gershon et al., Curr Opin Cell Biol, 20: 253-259, 2008; Smale et al., Cell, 57: 103-113, 1989).

Using the CapSeq protocol, it was found that, for CapSeq reads detected at a frequency of one read per 10 million total reads or greater, approximately 60% of the corresponding genomic loci exhibited a YR motif. This prevalence of the YR motif is significantly higher (P-value=0) than the 25% probability of a YR motif occurring by chance in the genome. The percentage of candidate TS sites with YR motifs increased to 80% when the cutoff was increased from 1 read per 10 million to 100 reads per 10 million, suggesting that reads lacking a YR motif represent either degradation-derived reads or TS sites that are less-frequently utilized. Using a cutoff of one CapSeq read per 10 million total reads and a requirement for a YR motif, the CapSeq data predicted approximately 64,000 candidate TS sites genome wide.

In order to pair candidate TS sites with existing annotations, CapSeq reads within a 1000 nt window upstream of gene annotations were considered. This distance was chosen as a conservative upper limit to allow for the possibility of non-annotated 5′ exon sequence or long distances between the TS site and the first splice acceptor site required for trans-splicing. For most genes the 3′ limit was arbitrarily set at a distance of 200 nt downstream of the annotated 5′ end. However, in order to reduce the chance of scoring degradation products as TS sites, the 3′ limit was reduced to 100 nt for very abundantly transcribed genes whose total read counts exceeded 1000 reads per ten million. Using these criteria, candidate TS sites could be assigned to more than 50% of annotations in WS215, including 52% of annotated protein-coding genes (10,667 genes), 15% of annotated pseudogenes (226), 54% of annotated non-coding RNAs (137), 74% of snoRNA loci (102), and 37% of snRNA genes (42). It was found that a typical gene has multiple candidate TS sites separated by several to sometimes hundreds of nucleotides (FIG. 16A, CapSeq panel), suggesting there is an inherent flexibility in transcription initiation mediated by RNA Pol II at these promoters and/or that multiple promoters exist at many of these loci.

19,828 candidate TS sites were also identified that could not be readily paired with annotations based on the above criteria. These defined 12,457 clusters where candidate TS sites were found to reside within a 100 nt interval typical of annotated Pol II genes. The majority of these (84%) were separated from other annotations or from each other by greater than 1 kb. These findings suggest that there are many as yet non-annotated Pol II loci in the C. elegans genome and/or that many of the existing annotations are separated from their actual 5′ ends by greater than the arbitrarily set 1 kb limit used for this analysis.

### Example 13

### Capped Small RNAs are Enriched Around TS Sites in C. elegans

Recent studies have identified csRNAs associated with promoters and TS sites (Haussecker et al., Nat Struct Mol Biol, 15: 714-721, 2008; Nechaev et al., Science, 327: 335-338, 2010; Seila et al., Cell Cycle, 8: 2557-2564, 2009; Taft et al., Cell Cycle, 8: 2332-2338, 2009). To identify csRNAs in C. elegans, an 18-40 nt size fraction was gel-purified from whole RNA. In order to select against the recovery of abundant un-capped small RNA species, including 22G-RNAs, miRNAs and mature piRNAs, the sample was treated with CIP to remove 5′ mono- or tri-phosphates, thus making them inaccessible to 5′ ligation (FIG. 14). The RNA was ligated with a linker at the 3′ end and gel-purified. Three fourths of the sample was then treated with TAP to decap csRNAs, thus exposing a 5′ monophosphate for 5′ ligation. The remaining one fourth of the sample was treated with polynucleotide kinase (PNK) to add a 5′-phosphate onto non-capped small RNA species. The CIP-TAP and CIP-PNK samples were then ligated with a 5′ linker, gel-purified, reverse-transcribed, and PCR-amplified.

As expected, deep-sequencing of the CIP-PNK sample revealed abundant 22G-RNAs, miRNAs and piRNAs, but very few small RNAs that mapped to Pol II promoters (FIG. 16A). In contrast, small RNAs upstream of Pol II loci were dramatically enriched in the CIP-TAP sample (FIG. 16A). After normalization to total non-structural reads that match the genome, candidate csRNA reads mapping within 1000 nt upstream of WS215 5′ end annotations (including 21U-RNA annotations, see below) were enriched 60-fold in the CIP-TAP sample relative to the CEPPNK sample (FIG. 16C). In contrast, miRNAs and mature 21U-RNAs were depleted ˜4-fold and 17-fold respectively in the CIP-TAP sample. The relative rate at which 22G-RNAs were recovered did not change significantly between the CIP-TAP and CIP-PNK samples. It was found that 42% of the sense-oriented csRNA reads identified in the CIP-TAP sample corresponded exactly to the 5′ ends of candidate TS sites identified in the CapSeq analysis (FIG. 16A), supporting the idea that CIP-TAP treatment enriches for C elegans TS-site-associated csRNAs.

Interestingly, bidirectional, divergent csRNAs were observed upstream of many genes (FIG. 16A).

At bidirectional loci, the antisense csRNAs were located an average of ˜150 nt upstream of the sense csRNAs. In contrast, long-capped RNA reads from CapSeq were almost exclusively collinear with the downstream gene (FIG. 16A). Altogether detected 73,984 reads were detected defining 10,296 distinct sense csRNA loci, and 24,691 reads defining 4,114 antisense csRNA loci. However, because the C. elegans genome is very compact and divergent genes are common, the above analysis may overestimate the number of antisense csRNAs. The sequencing depth of the CapSeq libraries was at least 10-fold greater than that of the CIP-TAP library. Therefore, the presence of numerous antisense-oriented csRNAs in the absence of corresponding longer CapSeq reads suggests that csRNAs are not directly processed or degraded from longer Pol II products (FIG. 16A).

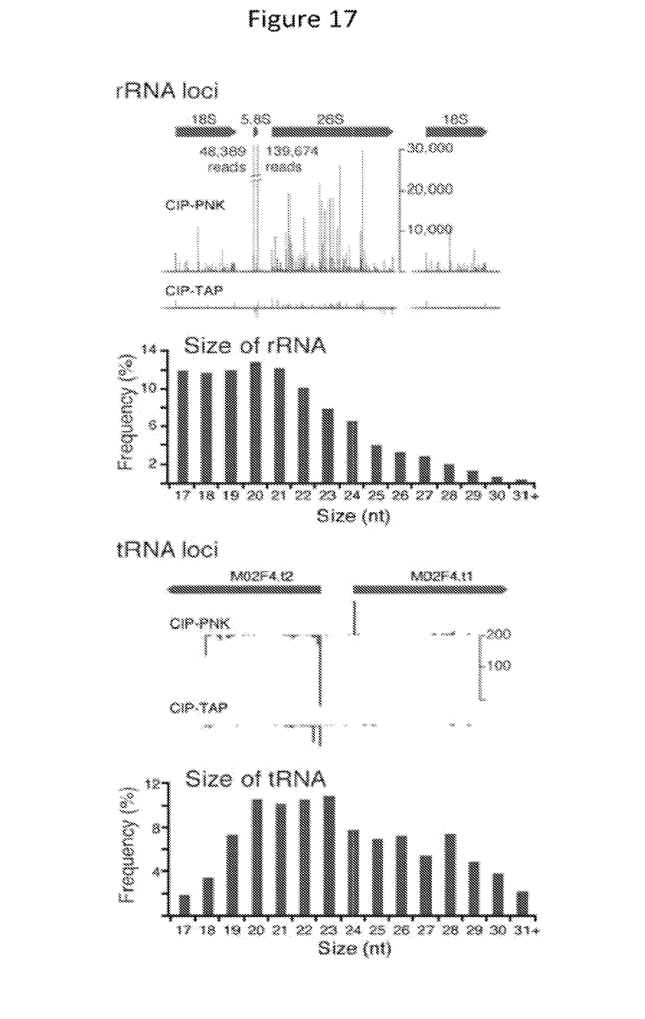

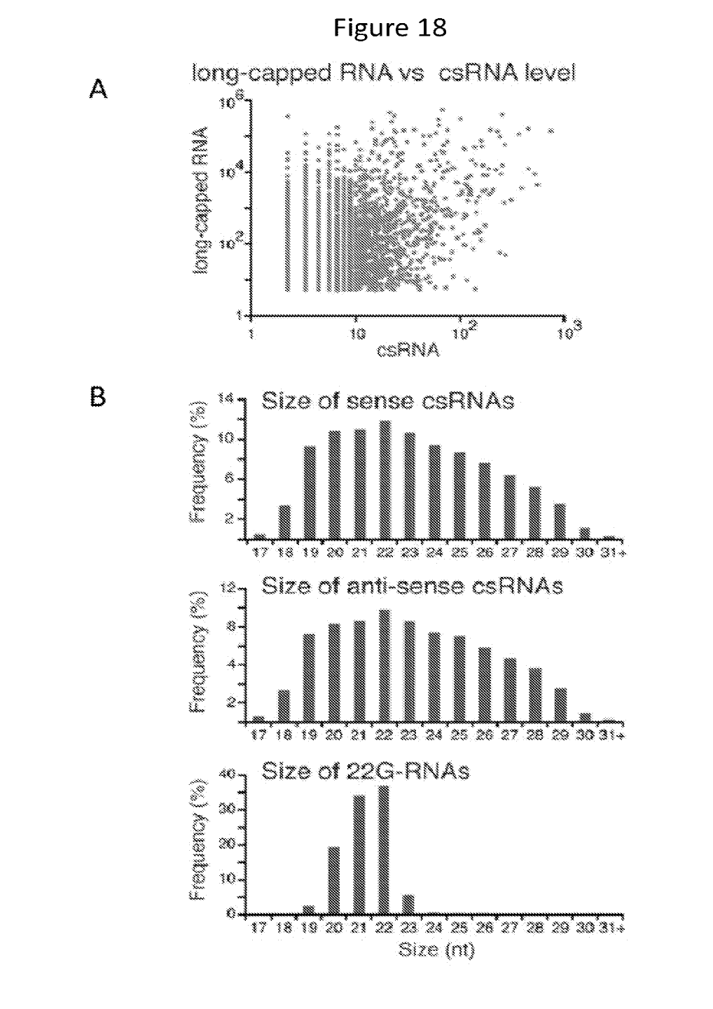

As with long capped RNAs cloned by CapSeq, csRNAs appeared to be specific to Pol II promoters, as they were not significantly enriched at Pol I or Pol III promoters (FIG. 17). At each locus, the 5′ ends of sense csRNAs were often coincident with the 5′ ends of long capped RNAs detected by CapSeq (FIG. 16A). In addition, the csRNAs and corresponding CapSeq reads were roughly proportional in abundance (FIG. 18A). However, csRNAs varied in length from 18 to 30 nts (FIG. 18B). Analysis of the nucleotide composition flanking the 5′ ends revealed that csRNAs, including upstream antisense csRNAs, exhibit a YR motif (FIG. 16B and FIG. 15). Together these findings suggest that csRNAs are associated with active C. elegans promoters and are likely to be independently transcribed by RNA Pol II.

The abundant 22G-RNAs of C. elegans are synthesized by RNA-dependent RNA polymerases (RdRP), utilizing the mature mRNA as a template (CIP-PNK sample in FIG. 16A; Claycomb et al., Mol Cell., 36: 231-244, 2009; Gu et al., Mol Cell. 36, 231-244, 2009). Interestingly, an analysis of the nucleotide composition flanking the 5′ ends of the 22G-RNA sequences revealed that RdRPs initiate at a YR motif (FIGS. 15 and 18B), in which R at position +1 strongly prefers a G. Moreover, we found that the extended motif yYRNyy was also associated with RdRP initiation. These findings suggest that Pol II and RdRPs initiate transcription at a similar motif but prefer distinct initiating purines in C. elegans.

### Example 14

### Identification of Primary miRNA TS Sites

miRNAs are sequentially processed from primary transcripts (pri-miRNAs) synthesized by Pol II. Drosha processes pri-miRNAs into stem-loop precursors (pre-miRNAs) that are exported to the cytoplasm and processed by Dicer into mature miRNAs (Hutvagner et al., Nat Rev Mol Cell Biol, 9: 22-32, 2008). To identify candidate TS sites for miRNA genes, both csRNAs and CapSeq reads mapping upstream of annotated miRNAs were analyzed. Because many miRNAs are co-expressed in a single primary transcript (Lau et al., Science, 294: 858-862, 2001), there are only about 100 unique miRNA loci annotated in the C. elegans genome. At least 1 candidate TS site was identified for 64 of the 100 annotated miRNA loci corresponding to 83 individual mature miRNAs.

It was found that CapSeq and csRNA reads that mapped upstream of the pre-miRNAs frequently shared the same 5′ end and, as with other Pol II loci, were often clustered within a short interval (FIG. 16D). Evidence was found for only a single group of TS sites upstream of each miR cluster, including the mir-54-56, mir-35-41 and mir-229/64-66 clusters (FIG. 16D), indicating that each cluster is co-expressed, as previously suggested (Lau et al., Science, 294: 858-862, 2001). Pri-miRNAs were rarely trans-spliced; a total of five SL-containing reads were associated with pri-miRNAs, and all of these were spliced to pri-let-7. These five SL-containing reads mapped ˜30 nt upstream of the Drosha-processed pre-let-7 RNA, while 20 non-SL reads mapped approximately 200 nt further upstream. Interestingly, it was found that some pri-miRNAs were expressed at levels comparable to the pre-mRNAs of common protein-coding genes, a finding that differs from previously published RNA-Seq data for miRNAs. Thus miRNAs appear to be conventional Pol II genes that are rarely trans-spliced.

TABLE 1
Comparison of CapSeq and published RNA-seq
reads that correspond to miRNAs. Each annotated
gene locus was extended upstream to include the capped
RNA loci, and then RNAs from CapSeq or RNA-seq were
mapped to each gene, as shown in column 2 and 3 respectively.
The relative ratio RNA-seq/CapSeq for each gene is shown in column 4.
miRNA levels in CapSeq vs. RNA-seq

| | | | |
|---|---|---|---|
| Gene | Capseq | RNA-seq | Ratio |

Protein Coding

| | | | |
|---|---|---|---|
| kin-31 | 3611 | 2021 | 1.79 |
| glc-1 | 176 | 1222 | 0.14 |
| rpl-12 | 217042 | 68267 | 3.18 |
| glh-1 | 10016 | 14267 | 0.70 |
| cel-1 | 1944 | 1621 | 1.20 |

miRNAs

| | | | |
|---|---|---|---|
| mir-35 | 7105 | 1 | 7105 |
| mir-61 | 911 | 0 | >1000 |
| mir-54 | 3890 | 7 | 556 |
| mir-58 | 2891 | 17 | 170 |
| mir-229 | 1548 | 45 | 34 |

### Example 15

### 21U-RNAs are Likely to be Individually Transcribed and Initiate 2 nt Upstream of the 5′ U

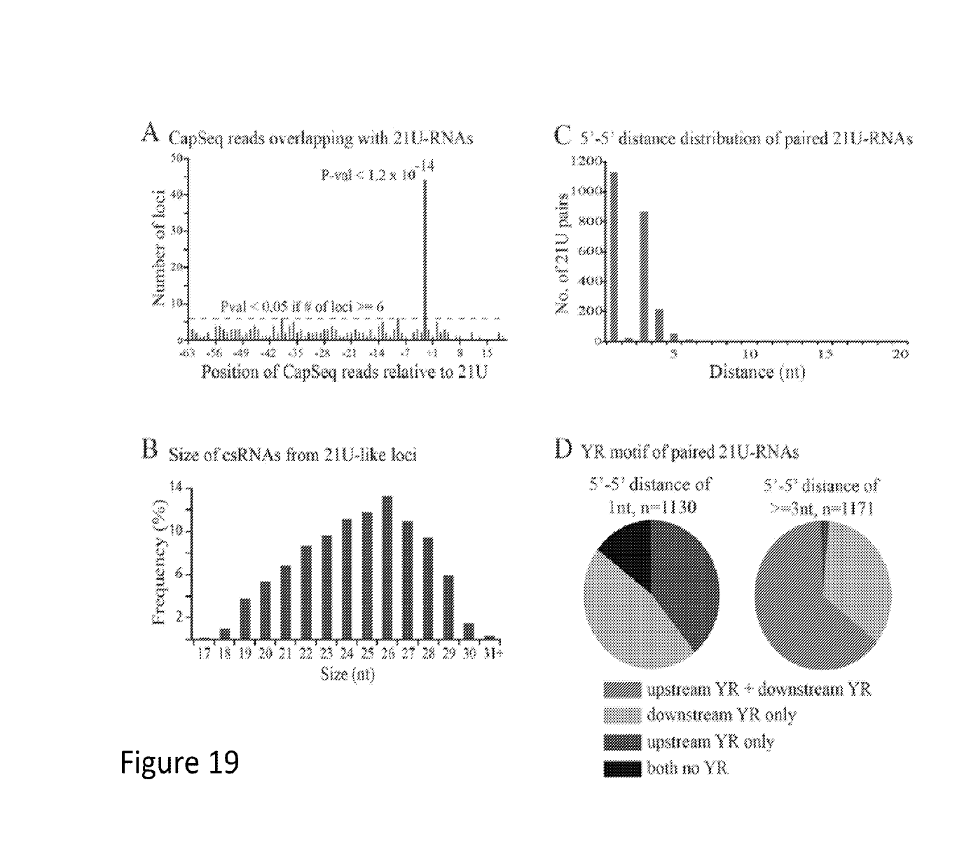

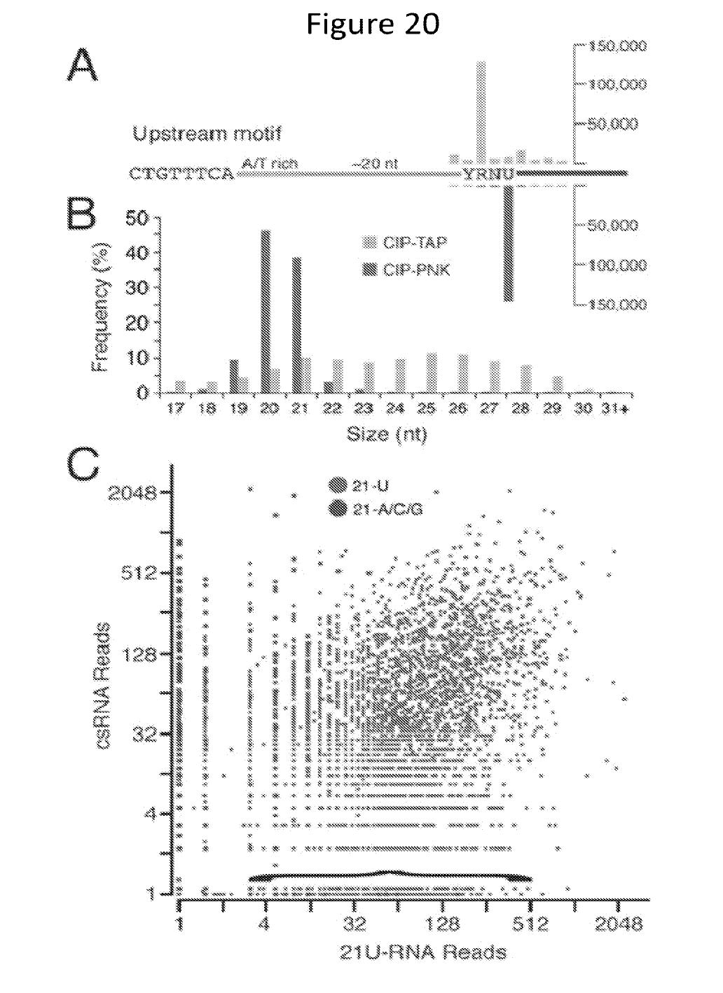

To identify potential precursor 21U-RNA transcripts (pre-21Us), a search was conducted for CapSeq and csRNA reads mapping to the 9079 unique, non-overlapping 21U-RNAs annotated in WS215. Strikingly, it was found that capped-short RNA reads identified by CIP-TAP were much more strongly correlated with 21U-RNAs than were capped long RNA reads identified by CapSeq. For example, CapSeq reads mapping to only 217 annotated 21U-RNA loci were identified, whereas candidate csRNAs mapped to approximately 6,000 of the 9,079 21U-RNA loci. Interestingly, a very strong bias for the mature 21U-RNA species to map 2 nt downstream of CapSeq loci was observed (44/217, p-value<1.2E-14; FIG. 19A and Table 2C) and csRNA loci (4,600/6,000, p-value=0; FIG. 20A).

Table 2C 21U-RNA related analyses. 21U-like RNAs, new 21U-RNAs, and 21U-RNAs with −2 CapSeq reads

Reads with 5′ ends that align 2 nt upstream of mature 21U-RNAs (−2 csRNAs) were enriched approximately 60-fold by CIP-TAP cloning relative to the CIP-PNK cloning method, and peaked at 25-26 nt (FIG. 20B). Moreover, for each locus, it was observed that the level of −2 csRNAs cloned by CIP-TAP was correlated with the level of mature 21U-RNAs recovered by CIP-PNK (FIG. 20C). Some loci that were annotated as highly-expressed 21U-RNA loci failed to produce detectable csRNAs (FIG. 20C, points lying just above the X axis). Visual inspection of several of these loci using Gbrowse revealed that they are likely to be derived from degraded 22G-RNAs that were mis-annotated as 21U-RNAs. Other loci with abundant csRNAs, but lacking mature 21U-RNA reads (FIG. 20C, points along the Y axis), define a set of “21U-like” loci previously annotated as PRG-1 associated RNAs and are discussed below.

Next, experiments were conducted to determine if partially overlapping 21U-RNA species are processed from a single or from multiple independent transcripts that share a promoter. Of approximately 6,000 annotated 21U-RNAs that partially overlap, a subset of 2,301 distinct 21U-RNA pairs were analyzed (FIGS. 19C and D; see Experimental Procedures at the end of Example 18). For 500 (22%) pairs, −2 csRNA reads were identified corresponding to both mature 21U-RNAs. For an additional 1,129 pairs (49%), −2 csRNA reads were detected that were associated with only one of the mature 21U-RNAs, corresponding to either the 21U-RNA encoded upstream (515 pairs, 22%) or downstream (614 pairs, 27%). Since the CIP-TAP data is far from saturated, these frequencies are clearly consistent with independent transcription of each pre-21U-RNA transcript produced at these tandem loci.

When the 2,301 21U-RNA pairs were analyzed for the presence of YR motifs, it was found that, for about half of the pairs (1,130), it is impossible for YR motifs to exist for both sister 21U-RNAs because the mature 21U-RNA 5′ ends are separated by only 1 nt (FIG. 19D). For these tandem 21U-RNAs, a YR motif was similarly associated with either the 21U-RNA encoded upstream (442 pairs, 39%) or downstream (523 pairs, 46%; FIG. 19D left panel). Importantly, this situation allowed for evaluation of the levels of both csRNAs and mature 21U-RNAs associated with a YR motif. Regardless of their arrangement, it was found that csRNAs were 10-fold more abundant for the YR-containing sister than for the non-YR-containing sister (paired t-test, p-value<0.0001). Similarly, the corresponding mature 21U-RNAs were approximately 10-fold more abundant for the YR-containing sister than for the non-YR-containing sister. Taken together, these findings strongly support the idea that, even within tandem 21U-RNA loci, the level of 21U-RNAs was positively correlated with that of the associated −2 csRNA. Thus the YRNT motif described by Ruby et al. (Cell, 127: 1193-1207, 2006) is a TS site for 21U-RNAs, where R (usually an A) is the +1 nt of a pre-21U and the +3 nt becomes the 5′ U of the corresponding mature 21U-RNA (FIG. 20A). These findings also suggest that the YR motif is strongly correlated with, but not essential, for Pol II transcription initiation.

### Example 16

### A+3 U is Required for piRNA Production or Stability

While analyzing the CIP-TAP data, it was noticed that there were many abundant csRNA-producing loci within the 21U-RNA clusters on LGIV for which mature 21U-RNAs were not detected. Altogether, 2,309 csRNA-producing loci were identified that fail to produce mature 21U-RNAs.

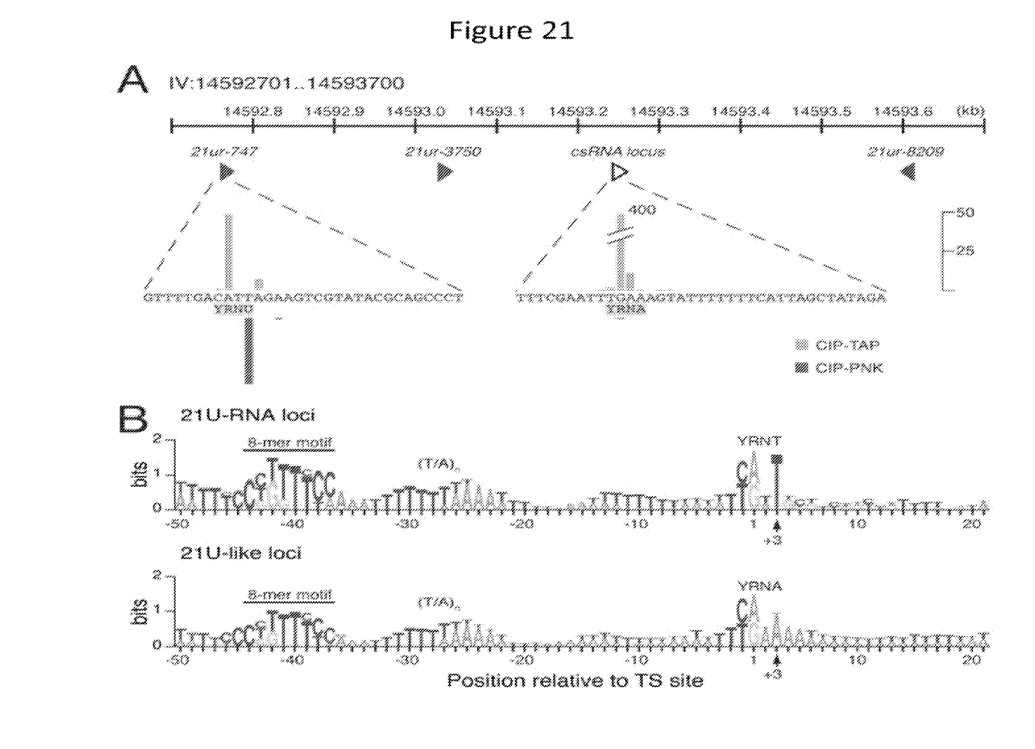

The csRNA reads obtained from these loci were similar in both size and abundance to those derived from canonical 21U-RNA loci (FIG. 21A and FIG. 19B). Furthermore, most of these loci (65%) exhibited the upstream 8 nt motif typical of canonical 21U-RNA loci with an adjusted motif score greater than 7 (Ruby et al., Cell, 127: 1193-1207, 2006). Interestingly, it was found that the csRNAs produced at these loci lack a +3U. The majority (−60%) contained a YRNA rather than the canonical YRNT motif (FIG. 21B). Previous studies identified approximately four hundred PRG-1-associated 21nt-RNAs (−3% of annotated 21Us; Batista et al., Mol Cell., 31: 67-78, 2008; Ruby et al., Cell, 127: 1193-1207, 2006). It was noted that ˜60% of these previously detected 21nt-RNAs exhibited corresponding csRNAs. Further examination of these 21U-like loci revealed that the mature 21nt-RNAs were on average at least 10-fold less abundant relative to their corresponding csRNAs (FIG. 21C, red) than were 21U-RNAs from canonical 21U-RNA loci (FIG. 21C, green). Taken together, these findings suggest that 21U-like loci express csRNAs at normal levels, but that the mature piRNAs are either inefficiently processed or unstable.

Given the large number of 21U-like loci, it was reasoned that polymorphisms in C. elegans wild-isolates might convert the +3 residue to a U at one or more of these loci. Consistent with this possibility two 21U-RNAs (IV+17159702-17159722 and IV+1590356315903583) were identified that were cloned from JU1580 (Felix et al., PLoS Biol, 9: e1000586, 2011) and CB4856 respectively, but not from N2, and that mapped to a 21U-like loci. In both cases, independent deep-sequencing data confirmed that the wild-isolates contain SNPs in the corresponding 21U-like loci that change the +3 residue of the csRNA to a U. Together, these findings indicate that a U at position +3 of a csRNA is important for 21U-RNA processing and/or stability.

### Example 17

### Capped Small RNAs Throughout the Genome are Processed into 21U-RNAs

In previous studies, the computational identification of 21U-RNAs required both the YRNT motif, which we now recognize as a transcription start site, as well as a larger motif containing a conserved 8 nt consensus (CTGTTTCA) positioned 40 nt upstream (FIG. 21B). This latter feature is absent upstream of the majority of csRNAs throughout the genome. Nevertheless, we noted that many csRNAs lacking the 8 nt motif exhibit a YRNT motif (FIG. 16B and FIG. 15), and thus contain a U at position +3. It was questioned whether this subset of csRNAs, which are associated with protein-coding and other Pol II RNA transcripts, might be processed into 21U-RNAs and loaded onto the Piwi Argonaute PRG-1. Indeed, a number of annotated 21URNAs coincide with csRNAs proximal to protein coding genes on chromosomes other than chromosome IV. To investigate this further, piRNAs enriched by PRG-1 immunoprecipitation (IP) relative to the input sample were deep sequenced. The new IP deep-sequencing data was consistent with the previously published and unpublished PRG-1 IP deep-sequencing data (Batista et al., Mol Cell., 31: 67-78, 2008). However, the CIP-PNK cloning used in the previous study generated much more noise from degraded mRNAs than did the TAP cloning used here (See Experimental Procedures at the end of Example 18). Altogether, 12,183 new 21U-RNA species were identified. For ˜30% of these corresponding −2 csRNAs were identified that were enriched in the CIP-TAP data sets.

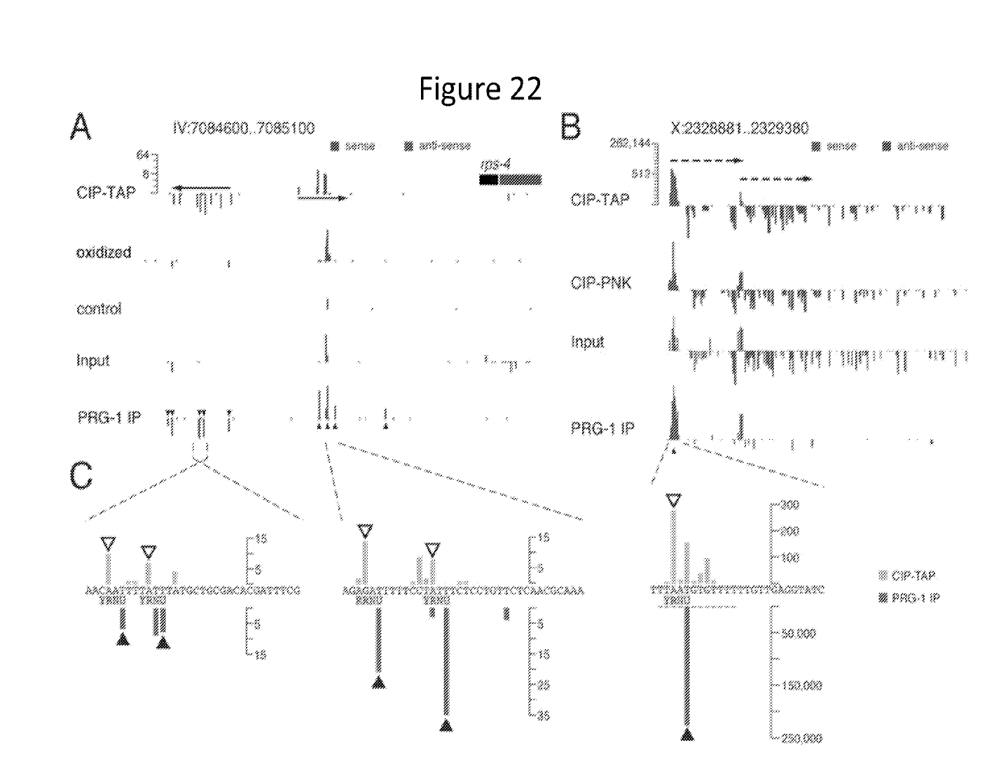

Although the majority of the newly defined 21U-RNAs were derived from atypical loci, it was found that they exhibit the same 2′-O-methyl modification found on the 3′ ends canonical 21U-RNA species, as they were enriched in a previously published 3′ end oxidization experiment (FIG. 22A; Vasale et al., Proc Natl Acad Sci USA., 107, (8):3582-7, 2010). Furthermore, like canonical 21U-RNAs, these new 21U-RNAs were only expressed in the germline, consistent with the germline-specific expression of PRG-1. Soma-specific loci, such as the gut-specific gene vit-1 that produced abundant csRNAs with a +3U did not give rise to 21U-RNAs.

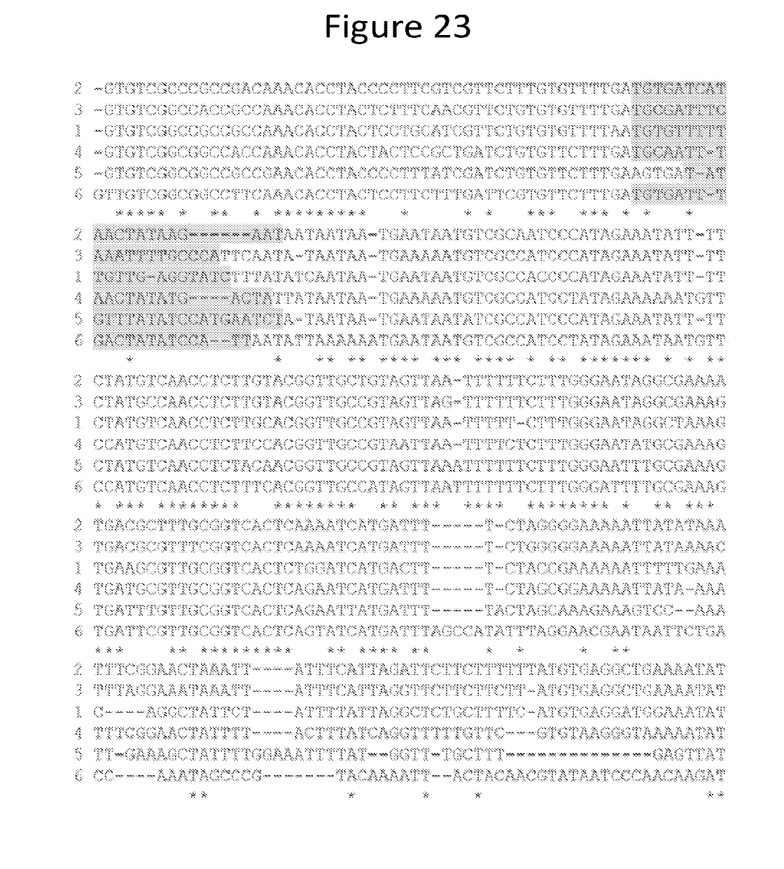

These newly identified 21U-species nearly double the total number of Piwi-associated small RNAs in C. elegans, and include several extremely abundant 21U-RNAs. The single most abundant 21U-RNA derives from a highly expressed csRNA locus on the X chromosome (FIGS. 22B and 22C). This X-locus is intriguing in that it has 5 homologs, also on X, all of which produce 21U-RNA reads which are not themselves conserved, and yet share extensive sequence identity both upstream and downstream (FIG. 23A). The trans-spliced leader (SL1) locus and several snRNA loci also produced very abundant 21U-RNA species. However, the majority of these atypical 21U-RNA loci produce relatively low levels of mature 21U-RNAs (Table 2B), and it was estimated that, collectively, this type of 21U-RNA species accounts for approximately 5% of total 21U-RNA levels (see Experimental Procedures at the end of Example 18).

### Example 18

### Mouse CapSeq

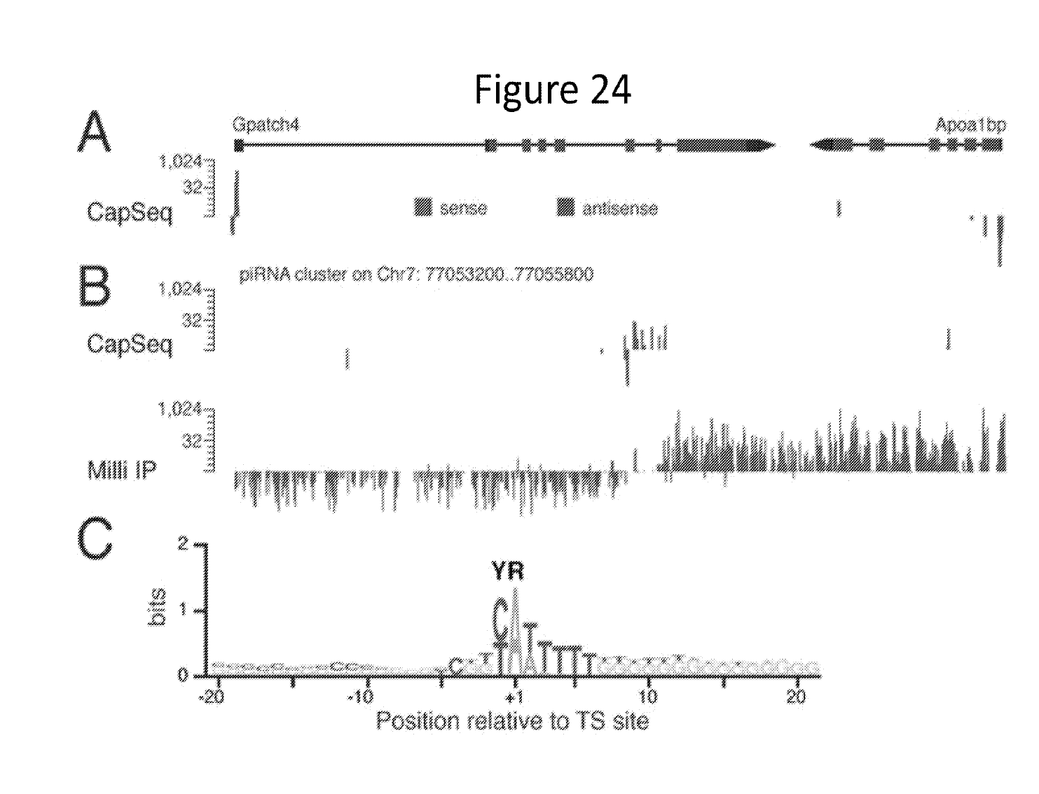

To confirm that CapSeq can identify TS sites from other species, a pilot study was performed using mouse testis RNA. As shown in FIG. 24A, it was found that CapSeq reads were strongly biased for the 5′ end of annotated genes. By searching for reads upstream of annotated miRNA loci, candidate TS sites were identified for hundreds of primary mouse miRNA genes (Table 2).

TABLE 2
Prediction of TS sites for mouse pri-
miRNAs. Predicted TS sites for mouse pri-miRNAs.
# The start sites of pri-miRNAs were predicted using CapSeq
# Predicted are the start sites for 134 individual miRNAs
# All coordinates are referred to Watson strand
# Lines starting with ‘>’ are the annotated pre-miRNAs
# For the annotated coordinates, ‘start’ and ‘end’ should be swiched if ‘strand’ is ‘−’
# For the mapped reads, ‘start’ is always the start site of a pri-miRNA.
# Format of the list:

| >miRNA | chromosome | strand | start | |
|---|---|---|---|---|
| chromosome | strand | start | reads | end |
| >miR-692-2 | 11 | + | 45643281 | 45643389 |
| 11 | + | 45642611 | 6 | |
| >miR-669a-1 | 2 | + | 10400938 | 10401034 |
| 2 | + | 10400329 | 64 | |
| >miR-199a-2 | 1 | + | 164147945 | 164148054 |
| 1 | + | 164147627 | 16 | |
| >miR-760 | 3 | − | 121996503 | 121996621 |
| 3 | − | 121996891 | 28 | |
| 3 | − | 121996892 | 13 | |
| 3 | − | 121996895 | 15 | |
| 3 | − | 121996889 | 5 | |
| 3 | − | 121996890 | 25 | |
| 3 | − | 121996888 | 20 | |
| >miR-669a-5 | 2 | + | 10422966 | 10423052 |
| 2 | + | 10422353 | 62 | |
| >miR-467a-5 | 2 | + | 10400425 | 10400507 |
| 2 | + | 10400329 | 64 | |
| >miR-1945 | 16 | − | 11254461 | 11254538 |
| 16 | − | 11254458 | 13 | |
| 16 | − | 11254463 | 6 | |
| 16 | − | 11254518 | 4 | |
| 16 | − | 11254460 | 19 | |
| 16 | − | 11254469 | 27 | |
| 16 | − | 11254456 | 5 | |
| 16 | − | 11254482 | 6 | |
| 16 | − | 11254477 | 5 | |
| >miR-5123 | 4 | − | 40797089 | 40797171 |
| 4 | − | 40797561 | 6 | |
| >miR-5133 | 9 | − | 61970325 | 61970401 |
| 9 | − | 61970333 | 5 | |
| 9 | − | 61970691 | 6 | |
| 9 | − | 61970329 | 7 | |
| >miR-466b-8 | 2 | + | 10422737 | 10422822 |
| 2 | + | 10422353 | 62 | |
| >miR-669a-9 | 2 | + | 10422966 | 10423052 |
| 2 | + | 10422353 | 62 | |
| >miR-669a-8 | 2 | + | 10422966 | 10423052 |
| 2 | + | 10422353 | 62 | |
| >miR-669a-12 | 2 | + | 10400942 | 10401028 |
| 2 | + | 10400329 | 64 | |
| >miR-467a-1 | 2 | + | 10400430 | 10400502 |
| 2 | + | 10400329 | 64 | |
| >miR-292 | 7 | + | 3219190 | 3219271 |
| 7 | + | 3218354 | 5 | |
| 7 | + | 3218787 | 5 | |
| >miR-5102 | 7 | + | 137977951 | 137978037 |
| 7 | + | 137977878 | 6 | |
| >miR-467a-4 | 2 | + | 10422449 | 10422531 |
| 2 | + | 10422353 | 62 | |
| >miR-291b | 7 | + | 3219483 | 3219561 |
| 7 | + | 3218787 | 5 | |
| >miR-195 | 11 | + | 70048544 | 70048637 |
| 11 | + | 70047701 | 5 | |
| >miR-467a-9 | 2 | + | 10422449 | 10422531 |
| 2 | + | 10422353 | 62 | |
| >miR-99b | 17 | + | 17967152 | 17967221 |
| 17 | + | 17966810 | 9 | |
| 17 | + | 17967083 | 5 | |
| 17 | + | 17967147 | 15 | |
| 17 | + | 17966856 | 5 | |
| >miR-466b-8 | 2 | + | 10400713 | 10400798 |
| 2 | + | 10400329 | 64 | |
| >miR-302d | 3 | + | 127248542 | 127248607 |
| 3 | + | 127247972 | 8 | |
| >miR-5125 | 17 | + | 23960258 | 23960336 |
| 17 | + | 23959697 | 5 | |
| 17 | + | 23959702 | 6 | |
| >miR-1948 | 18 | + | 12873320 | 12873404 |
| 18 | + | 12872645 | 5 | |
| >miR-669a-7 | 2 | + | 10400942 | 10401028 |
| 2 | + | 10400329 | 64 | |
| >miR-423 | 11 | − | 76891566 | 76891674 |
| 11 | − | 76891826 | 9 | |
| >miR-3960 | 2 | − | 32568420 | 32568492 |
| 2 | − | 32568989 | 25 | |
| 2 | − | 32569070 | 5 | |
| 2 | − | 32568991 | 16 | |
| 2 | − | 32568993 | 6 | |
| 2 | − | 32568992 | 25 | |
| 2 | − | 32568987 | 28 | |
| >miR-466b-7 | 2 | + | 10400714 | 10400801 |
| 2 | + | 10400329 | 64 | |
| >miR-669a-11 | 2 | + | 10422966 | 10423052 |
| 2 | + | 10422353 | 62 | |
| >miR-669a-10 | 2 | + | 10400942 | 10401028 |
| 2 | + | 10400329 | 64 | |
| >miR-466b-6 | 2 | + | 10422736 | 10422825 |
| 2 | + | 10422353 | 62 | |
| >miR-3471-2 | 4 | 139144791 | 139144892 | |
| 4 | − | 139145094 | 5 | |
| >miR-669a-4 | 2 | + | 10422966 | 10423052 |
| 2 | + | 10422353 | 62 | |
| >miR-92b | 3 | − | 89031038 | 89031120 |
| 3 | − | 89031263 | 5 | |
| 3 | − | 89031278 | 5 | |
| 3 | − | 89031260 | 9 | |
| 3 | − | 89031222 | 5 | |
| 3 | − | 89031286 | 13 | |
| 3 | − | 89031268 | 9 | |
| >miR-302b | 3 | + | 127248146 | 127248219 |
| 3 | + | 127247972 | 8 | |
| >miR-5134 | 17 | − | 24371472 | 24371549 |
| 17 | − | 24372279 | 5 | |
| >miR-345 | 12 | + | 110075183 | 110075278 |
| 12 | + | 110074246 | 4 | |
| >miR-5620 | 7 | + | 7251602 | 7251657 |
| 7 | + | 7250933 | 6 | |
| >miR-184 | 9 | − | 89697098 | 89697166 |
| 9 | − | 89697762 | 6 | |
| >miR-1906-2 | X | − | 86004813 | 86004892 |
| X | − | 86005590 | 40 | |
| X | − | 86005585 | 72 | |
| X | − | 86005581 | 33 | |
| X | − | 86005588 | 43 | |
| X | − | 86005572 | 14 | |
| X | − | 86005570 | 78 | |
| X | − | 86005587 | 52 | |
| X | − | 86005586 | 68 | |
| X | − | 86005589 | 28 | |
| X | − | 86005580 | 10 | |
| X | − | 86005591 | 8 | |
| X | − | 86005583 | 87 | |
| X | − | 86005582 | 21 | |
| X | − | 86005584 | 95 | |
| >miR-669a-12 | 2 | + | 10422966 | 10423052 |
| 2 | + | 10422353 | 62 | |
| >miR-467a-4 | 2 | + | 10400425 | 10400507 |
| 2 | + | 10400329 | 64 | |
| >miR-1906-1 | X | − | 86004813 | 86004892 |
| X | − | 86005590 | 40 | |
| X | − | 86005585 | 72 | |
| X | − | 86005581 | 33 | |
| X | − | 86005588 | 43 | |
| X | − | 86005570 | 78 | |
| X | − | 86005587 | 52 | |
| X | − | 86005586 | 68 | |
| X | − | 86005589 | 28 | |
| X | − | 86005580 | 10 | |
| X | − | 86005591 | 8 | |
| X | − | 86005583 | 87 | |
| X | − | 86005582 | 21 | |
| X | − | 86005584 | 95 | |
| >miR-1907 | 15 | − | 50720571 | 50720660 |
| 15 | − | 50720798 | 6 | |
| 15 | − | 50721509 | 9 | |
| >miR-5112 | 18 | + | 82889673 | 82889732 |
| 18 | + | 82889249 | 9 | |
| >miR-290 | 7 | + | 3218627 | 3218709 |
| 7 | + | 3218354 | 5 | |
| >miR-466b-1 | 2 | + | 10422740 | 10422821 |
| 2 | + | 10422353 | 62 | |
| >miR-466o | 2 | + | 10394167 | 10394250 |
| 2 | + | 10393469 | 5 | |
| >miR-302a | 3 | + | 127248414 | 127248482 |
| 3 | + | 127247972 | 8 | |
| >miR-298 | 2 | − | 174093005 | 174093086 |
| 2 | − | 174093375 | 28 | |
| 2 | − | 174093379 | 6 | |
| 2 | − | 174093376 | 13 | |
| 2 | − | 174093377 | 25 | |
| >miR-296 | 2 | − | 174092548 | 174092626 |
| 2 | − | 174093375 | 28 | |
| 2 | − | 174093379 | 6 | |
| 2 | − | 174093376 | 13 | |
| 2 | − | 174093377 | 25 | |
| >miR-467a-8 | 2 | + | 10400425 | 10400507 |
| 2 | + | 10400329 | 64 | |
| >miR-466b-4 | 2 | + | 10422736 | 10422825 |
| 2 | + | 10422353 | 62 | |
| >miR-467a-2 | 2 | + | 10422449 | 10422531 |
| 2 | + | 10422353 | 62 | |
| >miR-3084 | 19 | − | 60850232 | 60850300 |
| 19 | − | 60850785 | 5 | |
| >miR-125a | 17 | + | 17967776 | 17967843 |
| 17 | + | 17967611 | 5 | |
| 17 | + | 17967584 | 5 | |
| 17 | + | 17966856 | 5 | |
| 17 | + | 17967667 | 23 | |
| 17 | + | 17967647 | 15 | |
| 17 | + | 17967083 | 5 | |
| 17 | + | 17967147 | 15 | |
| 17 | + | 17967631 | 48 | |
| 17 | + | 17967615 | 9 | |
| 17 | + | 17967637 | 7 | |
| 17 | + | 17967634 | 8 | |
| 17 | + | 17967677 | 6 | |
| 17 | + | 17967639 | 13 | |
| 17 | + | 17966810 | 9 | |
| 17 | + | 17967605 | 5 | |
| 17 | + | 17967617 | 17 | |
| 17 | + | 17967593 | 24 | |
| 17 | + | 17967601 | 11 | |
| 17 | + | 17967603 | 5 | |
| 17 | + | 17967609 | 28 | |
| 17 | + | 17967629 | 28 | |
| 17 | + | 17967623 | 16 | |
| 17 | + | 17967600 | 6 | |
| 17 | + | 17967621 | 5 | |
| 17 | + | 17967659 | 6 | |
| >miR-5123 | 4 | − | 40797045 | 40797127 |
| 4 | − | 40797561 | 6 | |
| >miR-466b-5 | 2 | + | 10400714 | 10400801 |
| 2 | + | 10400329 | 64 | |
| >miR-22 | 11 | + | 75277218 | 75277312 |
| 11 | + | 75276663 | 6 | |
| 11 | + | 75276675 | 5 | |
| 11 | + | 75276715 | 28 | |
| 11 | + | 75276674 | 23 | |
| 11 | + | 75276754 | 7 | |
| 11 | + | 75276669 | 41 | |
| 11 | + | 75276671 | 9 | |
| >miR-320 | 14 | + | 70843317 | 70843398 |
| 14 | + | 70843311 | 5 | |
| 14 | + | 70843303 | 11 | |
| >miR-466b-2 | 2 | + | 10422740 | 10422821 |
| 2 | + | 10422353 | 62 | |
| >miR-683-1 | 13 | − | 50639995 | 50640103 |
| 13 | − | 50640826 | 13 | |
| >miR-1893 | 18 | − | 6490562 | 6490644 |
| 18 | − | 6490860 | 13 | |
| 18 | − | 6490803 | 21 | |
| 18 | − | 6490808 | 8 | |
| 18 | − | 6490794 | 8 | |
| 18 | − | 6490768 | 16 | |
| 18 | − | 6490795 | 13 | |
| 18 | − | 6490809 | 8 | |
| >let-7e | 17 | + | 17967316 | 17967408 |
| 17 | + | 17966810 | 9 | |
| 17 | + | 17967083 | 5 | |
| 17 | + | 17967147 | 15 | |
| 17 | + | 17966856 | 5 | |
| >miR-27b | 13 | + | 63402020 | 63402092 |
| 13 | + | 63401099 | 5 | |
| >miR-669a-9 | 2 | + | 10400942 | 10401028 |
| 2 | + | 10400329 | 64 | |
| >miR-669a-7 | 2 | + | 10422966 | 10423052 |
| 2 | + | 10422353 | 62 | |
| >miR-466b-2 | 2 | + | 10400716 | 10400797 |
| 2 | + | 10400329 | 64 | |
| >miR-2861 | 2 | − | 32568327 | 32568408 |
| 2 | − | 32568989 | 25 | |
| 2 | − | 32569070 | 5 | |
| 2 | − | 32568991 | 16 | |
| 2 | − | 32568993 | 6 | |
| 2 | − | 32568992 | 25 | |
| 2 | − | 32568987 | 28 | |
| >miR-669a-2 | 2 | + | 10422960 | 10423056 |
| 2 | + | 10422353 | 62 | |
| >miR-1956 | 3 | + | 138189385 | 138189449 |
| 3 | + | 138189367 | 5 | |
| 3 | + | 138189372 | 22 | |
| 3 | + | 138189356 | 10 | |
| 3 | + | 138189362 | 5 | |
| 3 | + | 138189365 | 5 | |
| 3 | + | 138189358 | 7 | |
| 3 | + | 138188614 | 5 | |
| >miR-1967 | 8 | + | 12654654 | 1126546622 |
| 8 | + | 126545832 | 6 | |
| >miR-466f-3 | 2 | + | 10393580 | 10393673 |
| 2 | + | 10393469 | 5 | |
| >miR-302c | 3 | + | 127248281 | 127248348 |
| 3 | + | 127247972 | 8 | |
| >miR-468 | 6 | − | 81846593 | 81846670 |
| 6 | − | 81847112 | 9 | |
| >miR-351 | X | − | 50406432 | 50406530 |
| X | − | 50407340 | 6 | |
| >miR-191 | 9 | + | 108470650 | 108470723 |
| 9 | + | 108470521 | 7 | |
| 9 | + | 108470110 | 22 | |
| 9 | + | 108469816 | 13 | |
| 9 | + | 108469939 | 192 | |
| 9 | + | 108470058 | 5 | |
| 9 | + | 108470020 | 11 | |
| 9 | + | 108469864 | 544 | |
| 9 | + | 108469891 | 9 | |
| 9 | + | 108469824 | 5 | |
| 9 | + | 108469848 | 8 | |
| 9 | + | 108469822 | 47 | |
| 9 | + | 108470145 | 10 | |
| 9 | + | 108470090 | 5 | |
| 9 | + | 108469870 | 11 | |
| 9 | + | 108470116 | 6 | |
| 9 | + | 108469821 | 13 | |
| >miR-369 | 12 | + | 110981628 | 110981706 |
| 12 | + | 110980732 | 19 | |
| >miR-467a-82 | + | 10422449 | 10422531 | |
| 2 | + | 10422353 | 62 | |
| >miR-125b-1 | 9 | + | 41390009 | 41390085 |
| 9 | + | 41389424 | 14 | |
| 9 | + | 41389422 | 10 | |
| >miR-669a-11 | 2 | + | 10400942 | 10401028 |
| 2 | + | 10400329 | 64 | |
| >miR-466b-7 | 2 | + | 10422738 | 10422825 |
| 2 | + | 10422353 | 62 | |
| >miR-680-2 | 1 | + | 103188376 | 103188485 |
| 1 | + | 103188109 | 7 | |
| >miR-669a-4 | 2 | + | 10400942 | 10401028 |
| 2 | + | 10400329 | 64 | |
| >miR-130b | 16 | 17124154 | 17124235 | |
| 16 | − | 17125146 | 36 | |
| 16 | − | 17124278 | 8 | |
| 16 | − | 17125143 | 87 | |
| >miR-669a-2 | 2 | + | 10400936 | 10401032 |
| 2 | + | 10400329 | 64 | |
| >miR-449c | 13 | + | 113826191 | 113826299 |
| 13 | + | 113825388 | 8 | |
| 13 | + | 113825385 | 9 | |
| 13 | + | 113825579 | 6 | |
| >miR-3971 | 11 | + | 75364931 | 75365013 |
| 11 | + | 75364555 | 5 | |
| >miR-199a-1 | 9 | − | 21300939 | 21301008 |
| 9 | − | 21301027 | 13 | |
| >miR-669a-8 | 2 | + | 10400942 | 10401028 |
| 2 | + | 10400329 | 64 | |
| >miR-669a-10 | 2 | + | 10422966 | 10423052 |
| 2 | + | 10422353 | 62 | |
| >miR-467a-5 | 2 | + | 10422449 | 10422531 |
| 2 | + | 10422353 | 62 | |
| >miR-466e | 2 | + | 10400715 | 10400798 |
| 2 | + | 10400329 | 64 | |
| >miR-466b-1 | 2 | + | 10400716 | 10400797 |
| 2 | + | 10400329 | 64 | |
| >miR-5136 | 19 | | 8963189 | 8963264 |
| 19 | − | 8963251 | 5 | |
| 19 | − | 8963195 | 14 | |
| 19 | − | 8963567 | 7 | |
| 19 | − | 8963198 | 5 | |
| >miR-669a-6 | 2 | + | 10422966 | 10423052 |
| 2 | + | 10422353 | 62 | |
| >miR-297a-2 | 2 | + | 10393881 | 10393970 |
| 2 | + | 10393469 | 5 | |
| >miR-5103 | 1 | − | 34489966 | 34490044 |
| 1 | − | 34490045 | 5 | |
| >miR-154 | 12 | + | 110976643 | 110976708 |
| 12 | + | 110975744 | 5 | |
| >let-7c-2 | 15 | + | 85537033 | 85537127 |
| 15 | + | 85536424 | 8 | |
| >miR-466b-4 | 2 | + | 10400712 | 10400801 |
| 2 | + | 10400329 | 64 | |
| >miR-669a-3 | 2 | + | 10400930 | 10401038 |
| 2 | + | 10400329 | 64 | |
| >miR-1949 | 18 | + | 35714221 | 35714290 |
| 18 | + | 35714145 | 5 | |
| >miR-484 | 16 | + | 14159719 | 14159785 |
| 16 | + | 14159184 | 7 | |
| 16 | + | 14159413 | 7 | |
| >miR-291a | 7 | + | 3218920 | 3219001 |
| 7 | + | 3218354 | 5 | |
| 7 | + | 3218787 | 5 | |
| >miR-466b-5 | 2 | + | 10422738 | 10422825 |
| 2 | + | 10422353 | 62 | |
| >miR-467a-2 | 2 | + | 10400425 | 10400507 |
| 2 | + | 10400329 | 64 | |
| >miR-1894 | 17 | + | 36054834 | 36054914 |
| 17 | + | 36053931 | 127 | |
| 17 | + | 36054010 | 13 | |
| >miR-3091 | 2 | + | 179992241 | 179992316 |
| 2 | + | 179992122 | 8 | |
| >miR-467a-1 | 2 | + | 10422454 | 10422526 |
| 2 | + | 10422353 | 62 | |
| >miR-467a-9 | 2 | + | 10400425 | 10400507 |
| 2 | + | 10400329 | 64 | |
| >miR-503 | X | − | 50407161 | 50407231 |
| X | − | 50407340 | 6 | |
| >miR-669a-6 | 2 | + | 10400942 | 10401028 |
| 2 | + | 10400329 | 64 | |
| >miR-34c | 9 | − | 50911139 | 50911215 |
| 9 | − | 50911938 | 6 | |
| 9 | − | 50912014 | 6 | |
| 9 | − | 50911934 | 9 | |
| 9 | − | 50912015 | 6 | |
| 9 | − | 50912009 | 13 | |
| 9 | − | 50911935 | 6 | |
| 9 | − | 50912012 | 23 | |
| 9 | − | 50912002 | 5 | |
| 9 | − | 50911959 | 13 | |
| 9 | − | 50911937 | 22 | |
| 9 | − | 50911940 | 7 | |
| 9 | − | 50912011 | 26 | |
| 9 | − | 50912013 | 15 | |
| 9 | − | 50912010 | 15 | |
| >miR-683-2 | 13 | − | 50639995 | 50640103 |
| 13 | − | 50640826 | 13 | |
| >miR-5105 | 5 | + | 147072579 | 147072660 |
| 5 | + | 147071832 | 5 | |
| >miR-23b | 13 | + | 63401792 | 63401865 |
| 13 | + | 63401099 | 5 | |
| >miR-466b-6 | 2 | + | 10400712 | 10400801 |
| 2 | + | 10400329 | 64 | |
| >miR-3474 | 2 | + | 158464319 | 158464376 |
| 2 | + | 158463395 | 11 | |
| >miR-425 | 9 | + | 108471108 | 108471192 |
| 9 | + | 108470521 | 7 | |
| 9 | + | 108470708 | 15 | |
| 9 | + | 108470110 | 22 | |
| 9 | + | 108471104 | 5 | |
| 9 | + | 108470145 | 10 | |
| 9 | + | 108470116 | 6 | |
| >miR-34a | 4 | + | 149442563 | 149442664 |
| 4 | + | 149441805 | 14 | |
| 4 | + | 149441806 | 5 | |
| 4 | + | 149441818 | 5 | |
| >miR-669a-5 | 2 | + | 10400942 | 10401028 |
| 2 | + | 10400329 | 64 | |
| >miR-301b | 16 | 17124493 | 17124589 | |
| 16 | − | 17125146 | 36 | |
| 16 | − | 17125143 | 87 | |
| >miR-467a-7 | 2 | + | 10400425 | 10400507 |
| 2 | + | 10400329 | 64 | |
| >miR-330 | 7 | + | 19766814 | 19766911 |
| 7 | + | 19766553 | 7 | |
| 7 | + | 19766559 | 21 | |
| >miR-412 | 12 | + | 110981499 | 110981578 |
| 12 | + | 110980732 | 19 | |
| >miR-497 | 11 | + | 70048219 | 70048302 |
| 11 | + | 70047701 | 5 | |
| >miR-467a-7 | 2 | + | 10422449 | 10422531 |
| 2 | + | 10422353 | 62 | |
| >miR-669a-1 | 2 | + | 10422962 | 10423058 |
| 2 | + | 10422353 | 62 | |
| >miR-367 | 3 | + | 127248651 | 127248725 |
| 3 | + | 127247972 | 8 | |
| >miR-409 | 12 | + | 110981368 | 110981446 |
| 12 | + | 110980732 | 19 | |
| >miR-466e | 2 | + | 10422739 | 10422822 |
| 2 | + | 10422353 | 62 | |
| >miR-34b | 9 | − | 50911667 | 50911750 |
| 9 | − | 50911938 | 6 | |
| 9 | − | 50912014 | 6 | |
| 9 | − | 50911934 | 9 | |
| 9 | − | 50912015 | 6 | |
| 9 | − | 50912009 | 13 | |
| 9 | − | 50911935 | 6 | |
| 9 | − | 50912012 | 23 | |
| 9 | − | 50912002 | 5 | |
| 9 | − | 50911959 | 13 | |
| 9 | − | 50911937 | 22 | |
| 9 | − | 50911940 | 7 | |
| 9 | − | 50912011 | 26 | |
| 9 | − | 50912013 | 15 | |
| 9 | − | 50912010 | 15 | |

Reads mapping to mouse piRNA clusters were also analyzed (FIG. 24B). Consistent with previous reports, it was found that multiple mouse piRNAs appeared to share a TS site and to be processed from a longer precursor RNAs (FIG. 24B). This contrasts with the findings for C. elegans where unique candidate TS sites were frequently detected at 2 nt upstream of individual mature piRNA species. Finally, the motif surrounding candidate mouse TS sites were analyzed and a clear YR motif was observed within a broader motif of YRNyy, in which R (usually an A) corresponds to the predicted 5′ nt (FIG. 24C). Thus these data show that CapSeq is generally useful for identifying Pol II TS sites and that C. elegans TS sites are similar to mammalian TS sites.

The data in Examples 10-18 interestingly show that the majority of 21U-RNA loci produce csRNAs but do not produce longer transcripts defined by CapSeq reads. The majority of 21U-RNA-associated csRNAs are unidirectional and originate precisely 2 nucleotides upstream of the corresponding mature 21U-RNA species. These findings suggest that csRNAs are processed into piRNAs by removing the cap plus two nucleotides, and by trimming the 3′ end. These data show that the 8 nucleotide motif upstream of canonical 21U-RNAs is not required for 21U-RNA biogenesis. Rather, these findings suggest that any germline-expressed csRNA that contains a +3U can give rise to a piRNA that is loaded onto the Piwi Argonaute, including thousands of csRNAs associated with protein-coding and other Pol II loci. These findings reveal a role for promoter-associated csRNAs in piRNA biogenesis and uncover a species of piRNA associated with TS sites of Pol II genes throughout the genome, nearly doubling the repertoire of piRNA species in C. elegans.

### Discussion of Examples 10-18

### C. elegans piRNAs are Expressed Individually by Pol II as Capped Short RNAs

The above examples inform the skilled artisan on several important aspects relating to the expression of C. elegans piRNAs. The analysis employed two approaches, CapSeq and CIP-TAP, both of which enrich for the 5′ ends of Pol 11 transcripts. The CapSeq protocol, designed to select for long-capped RNAs, identified only ˜200 sequences that overlap annotated piRNAs, but detected thousands of candidate TS sites for other Pol II genes. The CIP-TAP protocol, designed to detect capped-small RNAs, identified thousands of candidate piRNA precursor transcripts that average 26 nt in length and initiate 2 nt upstream of the mature piRNA species. In addition, CIP-TAP identified csRNAs that were associated with many other Pol II promoters, where they were frequently oriented divergently, with the sense csRNA often corresponding to a major TS site for the corresponding longer transcript detected by CapSeq.

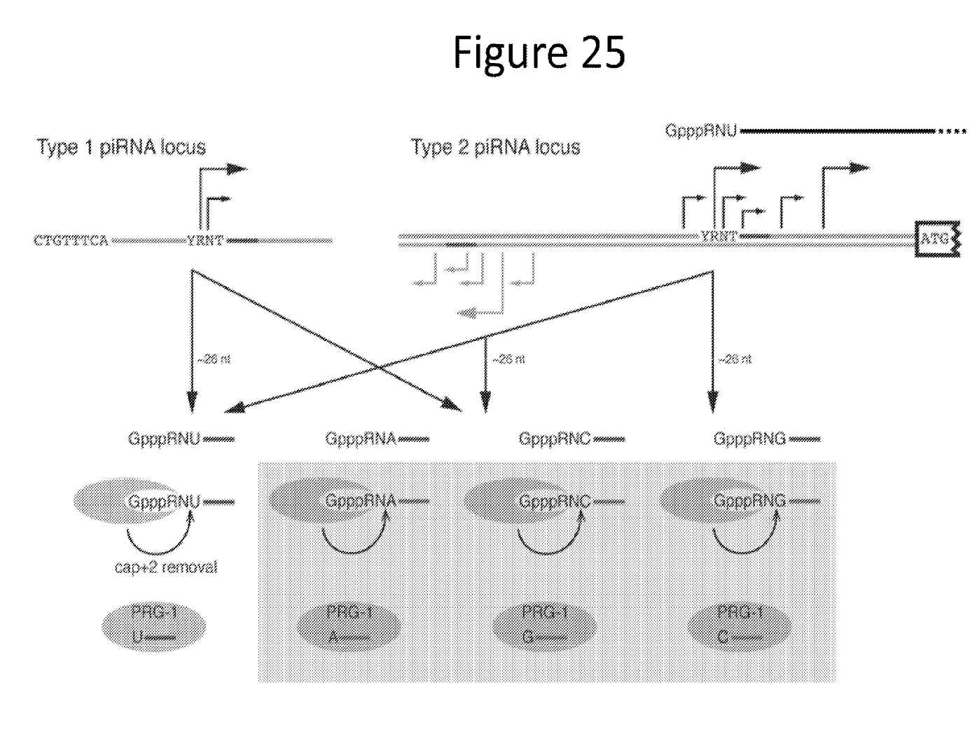

Strikingly, germline-expressed csRNAs genome-wide that contain a U at the +3 position were found to be processed into piRNAs (21U-RNAs) and to associate with the Piwi Argonaute PRG-1. These findings indicate that the U in the YRNU motif is important for 21U-RNA stability, processing, or Piwi Argonaute loading, and that the YR is important for efficient transcription initiation (See Model FIG. 25). Consistent with this idea, the distance between the conserved upstream 8 nt motif and the putative initiator element (YRNT) is similar to the distance between the TFIIB/TATA and the initiator elements of core TS sites described for other organisms (Juven-Gershon et al., Curr Opin Cell Biol, 20: 253-259, 2008). Based on these findings, it is proposed that C. elegans piRNAs can be divided into two categories (FIG. 25): Type 1 piRNAs, which correspond to the previously defined 21U-RNAs that share an 8 nt upstream motif and are clustered on chromosome IV (Batista et al., Mol Cell. 31: 67-78, 2008; Ruby et al., Cell, 127: 1193-1207, 2006); and Type 2 piRNAs, which need not have an 8 nt motif and are processed from csRNAs derived from the promoters of Pol II genes throughout the genome.

An Enzymatic Approach for 5′-End Anchored Transcription Profiling

Transcription profiling by deep sequencing has become an increasingly important tool for following gene expression. The CapSeq protocol described here facilitates transcription profiling by using a series of three enzymatic treatments that dramatically enrich for the 5′ ends of Pol II transcripts. Because this approach does not require affinity purification to remove structural RNA contaminants, it is usually performed on relatively small quantities of RNA, and aside from one gel purification for size selection, the entire procedure is carried out in a PCR tube. Importantly, the CapSeq procedure anchors clones at the 5′ cap of Pol II transcripts, and thus can clone RNAs with or without poly (A) tails. Thus CapSeq provides a quantitative way to profile a diversity of Pol II transcripts, while providing insights on alternative transcription-initiation sites, which may be of potential developmental significance.

The studies on C. elegans have enabled measurement of the fidelity with which CapSeq recovers the 5′ ends of transcripts. The presence of SL sequences on mRNA clones provides a convenient and objective marker for bona fide 5′ ends. By measuring the frequency of truncated clones lacking a full-length SL sequence, it was estimated that degraded mRNA clones were recovered in the CapSeq protocol at a frequency of less than one in 15,000 per position.

Genome-Wide Identification of Pol II TS Sites

The data described here provide the first systematic and comprehensive look at the TS sites of Pol II transcripts in C. elegans. The trans-splicing of SL sequences to the 5′ ends of many mature transcripts confounds the identification of TS sites in C. elegans. Consequently, only a handful of TS sites for C. elegans Pol II transcripts have previously been identified (Allen et al., Genome Res, 21: 255-264, 2011; Morton et al., RNA, 17: 327-337, 2011). By using CapSeq to clone capped transcripts from several different stages of development, candidate TS sites have been identified for approximately 50% of the annotated protein-coding genes in C. elegans. In addition, 5′ ends for other Pol II transcripts have been identified that are typically under-represented in poly(A)-selected RNA-seq studies, including snRNAs, snoRNAs, SL RNA precursors, and histone mRNAs. In keeping with predictions from previous studies (Allen et al., Genome Res, 21: 255-264, 2011), it was found that an overall 70% of annotated protein-encoding genes had trans-spliced forms. Because of the abundance of SL-containing reads, these findings provide a comprehensive measure of alternative spliced-leader usage for most genes and useful data for refining the prediction of SL splice-acceptor sites.

This analysis also enabled the identification of candidate TS sites for many miRNA primary transcripts. Altogether, by combining OP-TAP and CapSeq data, it was possible to predict TS sites for 60% of the annotated C. elegans miRNAs. Surprisingly, it found that expression levels, as inferred from read counts for pri-miRNAs, were comparable to that of many abundant protein-coding genes. In contrast, pri-miRNA transcripts were very rarely detected in data from a previous study that used poly(A) selection RNA-Seq protocols (Lamm et al., Genome Res, 21: 265-275, 2011), suggesting that either pri-miRNAs lose their poly(A) tail more rapidly than their 5′ cap, or perhaps lack a poly(A) tail entirely. It was concluded that CapSeq and CIP-TAP can be used to quantify the activity of a wide variety of Pol II genes. The approach described here can readily be extended to produce a comprehensive profile of C. elegans TS sites. Finally, sequencing of the mouse testes CapSeq library also revealed a strong enrichment for RNA 5′ ends and a YR motif around mouse TS sites of Pol II genes, including miRNAs and piRNAs. As such, it is predicted that CapSeq will be a valuable tool for the identification of Pol II TS sites, and for transcription profiling in a wide variety of organisms.

Capped Small RNAs are Associated with Promoters in C. elegans

csRNAs have thus been identified in C. elegans that map to TS sites of Pol II genes, including protein-coding, miRNA and other non-coding RNA genes. Like the longer reads recovered by CapSeq, csRNAs exhibit a consensus Pol II initiator element yYRyyy. Indeed the 5′ ends of csRNAs often coincide with the 5′ ends of CapSeq reads. However, unlike CapSeq reads, csRNAs are frequently bidirectional at promoters, with divergent csRNAs separated by an average of approximately 150 nt. This finding is consistent with the idea that many eukaryotic promoters are intrinsically bidirectional (Seila et al., Cell Cycle, 8: 2557-2564, 2009). In general, for csRNA and CapSeq reads that share a common 5′ end, the abundance of csRNA reads was proportional to the abundance of CapSeq reads, suggesting that csRNAs might be associated with Pol II initiation at active promoters. Despite their correlation with active gene expression, the above analysis suggests that csRNAs are relatively low abundance transcripts compared to other small RNAs. Based on the CIP-TAP cloning experiments, it is estimated that csRNAs represent less than 1% of the total small RNAs in adult C. elegans.

Capped-small RNAs that flank the TS sites of active promoters have been identified in mammals and Drosophila (Core et al., Science, 322: 1845-1848, 2008; Haussecker et al., Nat Struct Mol Biol, 15: 714-721, 2008; Seila et al., Science, 322: 1849-1851, 2008; Yamamoto et al., BMC Genomics, 8: 67, 2007). The above data suggest that csRNAs are most similar to PASRs, which were enriched using a CIP-TAP cloning method and had 5′ ends that frequently coincided with those of capped RNAs identified by CAGE (Affymetrix/Cold Spring Harbor Laboratory ENCODE Transcriptome Project, 2009; Kapranov et al., Science, 8, 316, 5830: 1484-8, 2007; Kapranov et al., Nature, 457, 7232:1028-32, 2009). Although the biogenesis and function of PASRs remains unknown, it has been speculated that PASRs might reflect Pol II pausing, or premature termination, or that they are processed from promoter-associated long-capped RNAs (Affymetrix/Cold Spring Harbor Laboratory ENCODE Transcriptome Project, 2009; Nechaev et al., Science, 327: 335-338, 2010). The above data suggest that csRNAs are independent transcripts. If csRNAs are derived from random 3′ degradation of long capped RNAs, it might have been expected to observe a broader size range continuing up to 32 nt (the largest size able to be sequenced in this experiment). The size of C. elegans csRNAs is similar to the estimated size of approximately 28 nts of nascent RNA that can be accommodated in the Pol II exit canal (Andrecka et al., Proc Nati Acad Sci USA, 105: 135-140, 2008; Chen et al., Proc Natl Acad Sci USA, 106: 127-132, 2009; Proudfoot et al., Cell, 108: 501-512, 2002). This size is also similar to that of csRNAs found associated with promoter-proximal pausing of Pol II, which occurs at many genes throughout metazoan genomes (Nechaev et al., Science, 327: 335-338, 2010; Rasmussen et al., J Mol Biol, 252: 522-535, 1995).

C. elegans piRNAs are Processed from Capped Small RNAs

Here it has been shown that csRNA loci genome wide can give rise to 21U-RNAs in approximate proportion to their abundance. The only requirement for 21U-RNA production was the presence of a U residue at the +3 position of the csRNA. These findings are consistent with a model in which csRNAs are precursors for 21U-RNA production (FIG. 25). The canonical 21U-RNA loci (Type 1 loci) appear to be specialized to produce csRNAs. The pattern of RNA expression at these loci was quite distinct from the pattern observed upstream of other Pol II transcripts. Type 1 21U-RNA loci typically produced abundant csRNAs and rarely, if ever, produced longer CapSeq reads (Table 3).

TABLE 3
The correlation of overall csRNAs with 21U-RNAs. ‘21U IV’,
all annotated 21U-RNAs in the two clusters on chromosome IV,
and ‘21U others’, all non-annotated 21U-RNAs outside the two
clusters above or on other chromosomes. For each group, mature 21U-
RNA, −2CapSeq (2nt upstream of mature 21U), and −2 csRNA were counted.
Analysis capped RNA reads of 21U-RNA loci

| | | −2 Reads | |
|---|---|---|---|
| | Mature 21U | | CIP- |
| | PRG-1 IP | Capseq | TAP |
| Chr IV | 2656058 | 523 | 174223 |
| Other | 167726 | 855855 | 47057 |

When multiple csRNAs were produced at 21U-RNA loci, they typically shared the same orientation and their 5′ ends were often separated by not more than 5 bp. In contrast, other Pol II loci, such as protein-coding genes, produced sense-oriented CapSeq reads at high abundance, and multiple relatively low abundance csRNAs that were often oriented in both directions, with the antisense csRNA divergently expressed about 150 nt upstream of the sense csRNAs (FIG. 25). These observations suggest that Type 1 21U-RNA loci somehow focus Pol II initiation and restrict elongation to promote csRNA biogenesis at the expense of longer transcripts. It is of interest to understand whether the upstream motif or other features of Type 1 21U-RNA loci govern their tendency to produce csRNAs but not longer Pol II transcripts.

### CONCLUSION

Recent studies have shown that PRG-1 and its piRNA cofactors provide an important first line of defense in a surveillance pathway that distinguishes self from non-self (Ashe et al., Cell, 150: 88-99, 2012; Lee et al., Cell, 150: 7887, 2012; Shirayama et al., Germline Cell, 150: 65-77, 2012). Importantly, PRG-1/piRNA complexes function in a context that does not require perfect base-pairing, greatly increasing the repertoire of potential target RNAs in C. elegans. The findings described here add to the amazing variety of piRNA biogenesis mechanisms, and identify a new type of piRNA that nearly doubles the number of piRNA species available for genome defense in C. elegans. The finding that promoter-associated small RNAs are processed and loaded onto an Argonaute also raises the intriguing possibility that Argonaute-small RNA pathways might regulate promoter activity directly.

### Experimental Protocols

### Examples 10-18

### Experimental Procedures

Worm Strains

The Bristol N2 strain of C. elegans was used in this study and cultured essentially as described (Brenner, Genetics, 77: 71-94, 1974).

RNA Cloning and Sequencing

RNA was extracted using TRI Reagent (MRC, Inc.) or phenol. For CapSeq, 0.5-2μ of total RNA was treated with Terminator exonuclease (Epicentre) to degrade rRNAs, calf intestine phosphatase (CIP, NEB) to remove 5′ phosphates, tobacco acid pyrophosphatase (TAP) to remove 5′ caps, and the resulting long-capped RNAs were ligated to a 5′ adapter. First strand cDNA was primed using a pool of random octamers containing a common 5′ sequence corresponding to the 3′ adapter oligo. The first strand cDNA was size selected and then amplified using Illumina adapter oligos. Details are provided below. Small RNA libraries were prepared essentially as described (Gu et al., Mol Cell., 36: 231-244, 2009), with some modifications. Briefly, gel-purified 18-40 nt small RNAs were dephosphorylated using CIP and then ligated to a 3′ adapter. The small RNAs were then treated with polynucleotide kinase (PNK, NEB), to add a 5′ monophosphate, or with TAP, to remove a 5′ cap and leave a 5′ monophosphate. The resulting RNAs were ligated to barcoded 5′ adapters and libraries were amplified using Illumina adapter oligos. Details are provided below. Libraries were sequenced using an Illumina Genome Analyzer II or HiSeq instrument at the UMass Medical School Deep Sequencing Core (Worcester, Mass.).

Bioinformatics

Sequences were processed and mapped to the genome using custom PERL (5.10.1) scripts, Bowtie 0.12.7 (Langmead et al., Genome Biol, 10: R25, 2009) and blastn (2.2.25). For C. elegans experiments, reads were aligned to the C. elegans genome (WormBase release WS215), Repbase 15.10 (Jurka et al., Cytogenet Genome Res, 110: 462-467, 2005), and miRBase 16 (Kozomara et al., Nucleic Acids Res 39: D152-157, 2011). For mouse cloning experiments, reads were aligned to the mouse genome assembly NCBIM37 (Ensembl 67), miRBase 18 and the non-coding RNA database fRNAdb 3.4 (Mituyama et al., Nucleic Acids Res, 37: D89-92, 2009). The Generic Genome Browser (Gbrowse 1.70, Generic Model Organism Database (GMOD)) was used to visualize the alignments.

Immunoprecipitation

The PRG-1 IP was performed as described previously (Gu et al., Mol Cell., 36: 231-244, 2009). Small RNAs were extracted from IP and input and cloned using a TAP cloning protocol, as described (Gu et al., Mol Cell., 36: 231-244, 2009).

Accession Numbers

Illumina data are available from GEO under the series number GSE40053.

CapSeq Protocol

To destroy 5.8S, 18S, and 26S rRNA, 0.5-2 μg of total RNA was treated with 0.1 U/μl Terminator exonuclease (Epicentre, TER51020) in 20 μl volume containing 1U/μl of SUPERase In™ (Ambion, AM2696) and 1× buffer A at 30° C. for 1-2 hr. The efficiency of the above reaction could be monitored by resolving the sample on a 5% denaturing PAGE gel visualized by Ethidium Bromide. To de-phosphorylate tRNA and 5S rRNA, the above reaction was diluted into 100 μl with 10 μl of 10× DNase I buffer, 20 U more of SUPERase In™, 30 U of CIP (NEB, MO290S), 5 U of DNase I (Ambion, AM2222), and H₂O, and incubated at 37° C. for 30 minutes. The RNA was extracted using phenol/chloroform with phase-lock column (5PRIM, 2302830), and precipitated with 0.3M NaAc (pH 5.2) and at least 1 volume of isopropanol plus 30 μg of glycoblue (Ambion, AM9515), a procedure called as RNA cleanup. To expose the 5′ phosphate in the cap structure, the RNA was treated with 0.25 U/μl TAP (tobacco acid pyrophosphatase, Epicentre, T19050) in 10 μl reaction containing 1U/μl SUPERase In™ and 1× TAP buffer at 37° C. for 1 hour, and then cleaned up, but without additional glycoblue because glycoblue was already added in the previous step.

The RNA was ligated with 5 μM of barcoded 5′ linkers in 10 μl reaction containing 1× buffer, 1 U/μl of SUPERase In™, 2 U/μl of T4 RNA ligase (Takara, 2050A), 10% DMSO, and 0.1 μg/μl BSA at 15° C. for 8 hours, and cleaned up without additional glycoblue.

To make cDNA, the precipitated RNA was annealed with 50 pmole of RT oligo plus 10 nmole of dNTP (each nucleotide) in a 13 μl reaction at 65° C. for 5 min, and then chilled on ice for at least 2 minutes. The sample was incubated with 5 U/μl Superscript III (Invitrogen, 18080), 10 mM DTT, 1 U/μl SUPERase In™, and 1× buffer, all of which brought in 7 μl volume to the previous annealing reaction. The RT was incubated at 15, 25, and 37° C. each for 15 min, and then at 50° C. for 30 min to finish the RT reaction. The RT was heat-inactivated at 85° C. for 5 min. To destroy RNA, 1 μl each of RNase A (Ambion, AM2270) and RNase H (Ambion, AM2292) is added to the RT reaction at 37° C. for 20 min. To increase the cDNA quantity (optional), the RT reaction was diluted into 100 μl PCR reaction containing 1× PCR buffer, 0.01 U/μl ExTaq (Takara, RROO1B), 0.05 μM of oligo CM013729, 0.2 mM dNTP, and H₂O, and a linear PCR was performed for 10 cycles with condition 94° C. 20 s, 55° C. 20 s, and 72° C. 30 s. The cDNA of desired size (here—130 to 170 nt for most samples or −50-130 nt for ya0217, including the linker), visualized using SYBRGold (Invitrogen, S-11494) with 5S and 5.8S RNA from the total RNA as size marker, was purified from a 15% denaturing PAGE gel. The cDNA was eluted using TE buffer (10 mM Tris pH 7.5, 1 mM EDTA) containing 0.3 M NaCl for 6 hours to overnight with constant vortexing, filtered through 0.45 μm Spin-x column (Costar, 19442-758), and precipitated with isopropanol and glycoblue, as above. The whole procedure after PCR above was defined as PAGE gel purification.

To obtain the optimal PCR cycle number to produce the cDNA, a testing PCR was performed in 50 μl reaction containing 1× buffer, 0.2 uM each of oligo CM013279 and solexa3sh, 20% of the eluted linear PCR product, 0.25 min dNTP and 0.025 U/μl ExTaq, for 15 cycles with condition 94° C. 20 s, 52° C. 20 s, and 72° C. 30 s. And then 5 μl each of oligo solexa3 and CM013278, each at 10 μM stock concentration, was added. 3 μl of PCR product was sampled at 3, 6, 9 and 12 more cycles, and then resolved on a 8% native PAGE gel visualized using Ethidium Bromide with 10 bp DNA marker (Invitrogen 10821-015). The optimal PCR cycle number was defined as the one at which the PCR reaction produces the cDNA of the desired size (here 130 to 170 nt for most samples or 50-130 for ya0217) without obvious bulged products (diffusive band running much more slowly). This was the condition to make the final cDNA amplicons.

To check the quality of the library, TA cloning followed by colony PCR was performed to obtain individual cDNA species sequenced using the traditional method. Finally, single end 76 or 100 nt sequence was obtained using Illumina Genome Analyzer or Hi-Seq.

Oligo Used in CapSeq:

RT oligo:
CAGAAGACGGCATACGANNNNNNNN
(N, random nucleotide)
PCR oligo:
solexa3:
CAAGCAGAAGACGGCATACGA
solexa3sh:
GCAGAAGACGGCATACGA
CM13279:
GTTCTACAGTCCGACGATC
CM13278:
AATGATACGGCGACCACCGACAGGTTCAGAGTTCTACAGTCCGACG
ATC 5′
linker: DNA/RNA hybrid oligo
(RNA preceded with ‘r’, otherwise DNA)
TCTAC-rArGrUrCrCrGrArCrGrArUrC-barcode
Barcodes:
A-rTrGrArC, B-rCrArGrT, C-rGrCrTrG, D-rArTrCrA

Small RNA Cloning:

A ligation dependent method was used, as described (Gu et al., Mol Cell., 36: 231-244, 2009) to make small RNA libraries. PRG-1 IP was performed using the same antibody and method, as described (Batista et al., Mol Cell., 31: 67-78, 2008; Gu et al., Mol Cell., 36: 231-244, 2009). Unlike CIP-PNK cloning or ligation independent cloning, TAP cloning, which clones much less degradation products, was used to clone RNA from PRG-1 IP, the oxidized sample for enriching for the 3′ modified RNA (Gu et al., Mol Cell., 36: 231-244, 2009), and the corresponding control samples. To clone capped small RNA, 18-40 nt RNA was first purified from 40 μg of total RNA using a 15% denaturing PAGE gel, and then treated with 0.5 U/μl CIP in 100 μl reaction containing 0.5 U/μl SUPERase In™ and 1×CIP buffer at 37° C. for 1 hour. The dephosphorylated RNA was cleaned up, ligated with a 3′ linker, gel-purified, and split into two parts: one quarter treated with PNK and the rest treated with TAP. The treatment and the following cloning protocol were described previously (Gu et al., Mol Cell., 36: 231-244, 2009). Except the previously published oxidized sample (the first step of beta-elimination) and its control, other samples were cloned using 5′ 4nt-barcoded linker. Single-end 36nt sequence was obtained using illumina genome analyzer.

Bioinformatics Analysis:

Genome and annotations used include WormBase release WS215, Repbase 15.10, and miRBase release 16 for C. elegans, and NCBIM37.67, miRBase 18 and non-coding RNA database fRNAdb 3.4 for mouse. Bowtie 0.12.7, blastn 2.2.25 and custom PERL (5.10.1) scripts were used to map and analyze the sequence. Gbrowse 1.70 was used to visualize the alignments. In general, all analyses were performed using custom PERL scripts.

A. Mapping CapSeq Reads to C. elegans Genome

Single-end reads of 75 nt (L10831, L30831 and YA0831) or 100 nt (avr0217 and ya0217) were obtained using Solexa Genome Analyzer and Hi-Seq system respectively. Reads were debarcoded, and then trimmed off the 3′ linker starting with TCGTATGCC. If there was no such sequence, an incomplete 3′ linker (TCGTATGC, TCGTATG, TCGTAT, TCGTA, TCGT, TCG or TC) at the very 3′ end was searched and removed in a non-recursive way. To avoid the mutations introduced by random priming in RT, the last 8 nt of the read was further trimmed off after removal of the 3′ linker. The processed RNA read with size of at least 17 nt was subject to a custom PERL script to identify and remove the splicing leader or its variants with up to 2 mutations. Then the non-SL-containing part was mapped to C. elegans genome WS215 and annotations using Bowtie 0.12.7 with parameters: -n 2-e 180 -a --best --strata -m 200. ‘-e 180’ defines the maximum mutations allowed, here 6; ‘-a --best --strata’ only returns the best matches; and ‘-m 200’ only reports RNA read with less than 200 best matches. The mutation rate allowed for the alignment was 0 for reads 17-19 nt long, 1 for 19-21 nt, 2 for 22-24 nt, 3 for 25-49 nt, 4 for 50-74 nt, and 5-6 for >=75 nt. The unmatched RNA reads of size >=40 nt were mapped to genome WS215 partially using blastn with parameter e 0.01. A PERL script was used to search the unaligned part of the RNA within 1000 nt flanking the reads which satisfied the splicing criterion, and only the best exon-exon candidates were obtained. A PERL script was used to obtain the histogram of the start loci of mapped reads, after being normalized to 10 million of total reads mapped to the sense strand of protein coding genes.

B. Mapping Small RNA and RNA-Seq to C. elegans

The C. elegans RNA-seq data using directional cloning strategy was obtained from GEO GSE22410 (Lamm et al., Genome Res, 21: 265-275, 2011). In other cases, a custom PERL script was used to de-barcode the sample and remove the 3′ linker CTGTAG and beyond. If the reads do not contain a complete 3′ linker, the incomplete 3′ linker CTGTA/CTGT/CTG/CT at the very 3′ end was removed in a non-recursive way. RNA at least 17 nt was used in the following analysis.

Bowtie 0.12.7 was used to match RNA to the annotations and genome with parameter -v 3 -a --best --strata -m 400. A custom PERL pipeline was used to perform the post-matching analysis, and the mutation rate allowed for RNA at least 17 nt long was: 0 mismatch for size 17-18nt, 1 for 19 to 21 nt, 2 for 22 to 24 nt, and 3 for longer than 24 nt. The bowtie parameter ‘-a --best strata’ only reports the best matches. If an RNA sequence was mapped to several genomic loci, then the reads of the RNA were split evenly to each locus. To account for the different sequencing volume, the small RNA data was normalized to 5 million of non-structural reads mapped, and the RNA-seq (mRNA) data was normalized to 10 million of sense protein coding reads. A PERL script was used to draw the scatter plot in FIG. 3B, and all other alignment figures were generated using Gbrowse 1.70. The single nt histogram for the start sites of mapped reads was obtained using a custom PERL script and Gbrowse.

Analysis of C. elegans CapSeq Quality-Enrichment for Capped RNA

To estimate the enrichment for reads mapped to the very 5′ end (long capped RNA), relative to those mapped to the rest of the genes (potentially non-capped but cloned anyway), two analyses were performed.

In analysis 1, five annotated SL1-containing genes, alh-8, rps-24, rps-3, rps-29 and rps-15 were used to identify and quantify reads perfectly matched to the first 30 nt of each gene plus the complete or partial SL1. Each sample was normalized to 10 million of sense protein coding reads. Because debarcoding allowed one mutation, SL1 missing the first nucleotide (position 21 relative to the last nt of SL1) could be caused by debarcoding, thus ignored. It was also required that there be at least 3nt for the identity of SL1. Therefore, only positions 3-20, relative to the last nt of SL1, were used to calculate the degradation rate for each position of SL1, 1/15297 on average.

In analysis 2, only considered 6967 of the annotated trans-spliced genes were considered which were also confirmed by the data described herein. All sense reads mapped to these protein coding genes were obtained, and then the number of SL-containing reads (T-SL), which represented some, if not all, of the long capped RNA reads, was compared with the number of non-SL-containing reads (T-non-SL), which could be non-capped RNA or non-trans-spliced long capped RNA reads. Assuming all the non-SL-containing reads as non-capped reads, the relative enrichment for capped reads is equal to (T-SL/T-non-SL)*1500. ‘1500’ is the average gene size. This resulted in ˜17,000 fold enrichment for full-size SL1 reads (position 22 relative to the last nt) over any other positions, a rate consistent with that in analysis 1. Analysis 2 would underestimate the enrichment ratio because some non-SL-containing reads are capped RNAs.

Mapping SL-Containing Reads to Protein Coding Genes

CapSeq samples included were avr0217, L10831, L30831, ya0217, and YA0831. Reads mapped to tRNAs and rRNAs were removed completely. SL-containing, uniquely matched, and 5′ perfectly matched RNAs with size of at least 20 nt after removal of SL was used in this analysis. For each mapped locus, represented by the start site of the mapped RNA, the nearby loci from 1 nt upstream to 1 nt downstream were scanned, and then the original locus was removed if at least one of the nearby loci had 10 fold more reads mapped. RNA reads were first normalized to 10 million of sense protein coding reads, and a histogram for the start sites of mapped RNAs was generated for each sample. The five histograms were combined and loci with less than 1 read were removed.

Every genomic position within a gene was assigned with a type, including start, exon and intron. For a gene containing several transcripts, the type ‘start’ had priority over ‘exon’, and ‘exon’ had priority over ‘intron’, if a position was associated with multiple types. To associate an SL-containing RNA with a gene, a PERL script searched within the annotated genes. If failing, the script then searched for the nearest transcripts within 1-500nt downstream the start site of the mapped RNA. If still failing, the script searched within 1-500 nt upstream of the start site. If still failing, the script labeled the start site of this RNA as ‘NA’, meaning it cannot find a gene associated with the RNA.

Identification of Pol II Start Sites for Protein Coding Genes Using CapSeq

CapSeq samples included were avr0217, L10831, L30831, ya0217, and YA0831. After removal of reads mapped to the structural RNAs, only uniquely mapped, 5′ perfectly matched, and non-SL containing reads with size >=30 nt were included in the following analysis. RNA reads were first normalized to 10 million of sense protein coding reads and a histogram for the start sites of mapped reads was generated for each sample. The histograms were combined for the five samples, and loci with less than 5 reads were removed. 944 genes, each with 1000 or more reads out of ˜7 million of sense reads in YA0831, were defined as the top genes. For these genes, only loci mapped within a 200 nt region from 100 nt upstream to 100 nt downstream the annotated start sites were considered, because the read number cutoff 1/10 million for the start loci could bring in some degradation products from genes with the most abundant reads. For each locus, the nearby loci from 1 nt upstream to 1 nt downstream were scanned, and the original locus was removed if at least one of the nearby loci had at least 10 fold reads mapped.

Then a script searched the nearest gene for each locus within a range from 200 nt upstream to 1000 nt downstream. If the start site of a transcript was covered by at least some RNA reads mapped, the transcript was labeled as ‘yes’ for coverage by CapSeq. Or it was labeled as ‘NA’ or Not′ in the last column. The final output position of the CapSeq start locus was relative to the start site of the transcript assigned as described above, with negative number indicating upstream.

To analyze the motif around the start site, nt occurrence from −50 (upstream) to +50 around each start locus was summed up. The weight of each start site was set up as 1, rather than the mapped reads, thus avoiding bias caused by loci with the most abundant reads.

Identification of csRNAs Upstream Protein Coding Genes

Sense csRNAs and anti-sense csRNAs were defined separately using CIP-TAP sample. Background noise filtered included reads mapped to sense 21U-RNAs, sense/anti-sense reads mapped to tRNAs/rRNAs/miRNAs, anti-sense reads mapped to other structural RNAs such as snRNAs/snoRNAs, and anti-sense reads mapped to protein coding genes/pseudogenes. Only uniquely matched reads outside the two chromosome IV regions with canonical 21U-RNAs were considered. Furthermore, the included reads were enriched at least 10 fold in CIP-TAP sample, as compared to those in CIP-PNK sample, and had at least 2 reads per 5 million of non-structural reads in CIP-TAP. A script searched sense csRNAs within a region from 500 nt to 1 nt upstream any SL-containing transcripts or from 50 nt upstream to 50 nt downstream any non-SL-containing transcripts, and stops if reaching the upstream genes in the same direction.

To identify the anti-sense csRNAs, the 200 nt region from 100 to 300 nt upstream every sense csRNA was scanned, and the scanning process stopped before the upstream transcripts.

Analysis of the Motif and Size of 22G-RNA

Included in this analysis were anti-sense RNAs mapped to protein coding genes with 1^{st} nt perfectly matched and at least 1 per million non-structural reads in CIP-PNK sample. In the motif analysis, the position was referred to the start site of mapped 22G-RNA locus with negative number indicating upstream positions.

Analysis of the Expression Levels of csRNAs and lcRNAs at TS Sites

Sense csRNAs and lcRNAs at TS sites for protein coding genes were compared, and then the 4344 loci having both csRNAs and lcRNAs were used to draw a scatter plot.

Analysis of the Ratio of Trans-Spliced Genes in C. elegans Genome

The number of SL-containing genes and the number of non-SL-containing genes were obtained from the above analysis 4 and 5. The total number of genes mapped with or without SL was 14337, among which 10276 genes had 5′ end reads with SL, 10679 genes had 5′ end reads without SL, and the two datasets shared 6618 genes.

Analysis of csRNA Derived from miRNA Loci

All miRNA loci were visually checked to find upstream csRNA loci using Gbrowse, and 51 csRNA regions upstream miRNAs were hand-picked. RNA reads uniquely mapped to these loci were normalized to 5 million of non-structural reads. A histogram of the RNA start site was generated. Then removed were start sites with less than 2 reads or with nearby start sites (−2 to 2) having more than 5 fold reads. These start sites were used in the motif analysis, and the reads mapped to these sites were used in the size analysis.

Prediction of the Pri-miRNA Start Sites

All miRNA loci were visually screened for regions usually less than 500 nt starting upstream individual miRNAs or clusters of miRNAs, which contained both CapSeq or csRNA loci. 66 of such regions were hand-picked from Gbrowse. Only uniquely mapped RNAs without any mutation at the first nucleotide were used. Included in the analysis were the five Capseq samples aforementioned, each of which was normalized to 10 million of sense protein coding reads, and the CIP-TAP sample, which was normalized to 5 million of non-structural RNAs. A combined histogram of CapSeq start sites was generated and any start loci with less than 1 reads/10 million sense protein coding reads was removed. A histogram of CIP-TAP start site was also generated, and any start site with less than 1 read per 5 million reads was removed. For each miRNA, the most abundant start locus with YR motif (R, the first nt of the mapped RNAs) from either histogram was assigned as the start sites for the pri-miRNA respectively. If several loci were identified from the same histogram, the closest locus was selected. After obtaining both start sites for each pri-miRNAs, one from CapSeq and the other from CIP-TAP sample, the distance between the two sites was analyzed.

Comparison of CapSeq and RNA-Seq

In this comparison, we randomly chose 5 non-SL-containing protein coding genes with size of 2156, 1573, 2560, 1382, and 860 nt for kin-31, glc-1, rpl-12, glh-1, and cel-1, and 5 miRNAs, each of which represent the first miRNA, if in the miRNA clusters, with genomic size of 897, 941, 580, 548, and 402 nt for mir-35, mir-61, mir-54, mir-58, and mir-229. The above strategy eliminates the complexity caused by trans-splicing and dramatically reduces the size difference between individual mRNAs and miRNAs. Using CapSeq and CIP-TAP data visualized by Gbrowse, we expanded the coding region of each miRNA or mRNA, as annotated in WS215, to include the upstream capped RNA region. Only uniquely mapped reads with size of >=30 nt falling within the desired region were considered in the analysis. 11 samples of directional RNAseq (Lamm et al., Genome Res, 21: 265-275, 2011), and 5 samples of CapSeq were combined respectively. In this way, both RNA-seq and CapSeq were considered as mixed stage samples because at least both contain L1, L3 and YA. CapSeq reads basically represented capped RNAs, and RNA-seq RNAs mostly represented non-capped RNAs. Assuming there was no difference between RNA enrichment methods used in CapSeq (enzymatic ribo-minus) and RNA-seq (poly A selection), the capped RNAs and non-capped RNAs should correlate positively, regardless of the source, here miRNAs or mRNAs. However, miRNAs as a whole were almost completely depleted from RNA-seq, as compared to mRNAs, strongly suggesting that the enrichment method could cause this bias.

The coordinate uses for each gene is as below:

| | | | | |
|---|---|---|---|---|
| cel-1 | I | 9665200 | 9668655 | + |
| glc-1 | V | 16219500 | 16221896 | + |
| glh-1 | I | 6854600 | 6857536 | + |
| kin-31 | III | 8671550 | 8673545 | + |
| rpl-12 | IV | 13239564 | 13240700 | − |
| mir-229 | III | 2172300 | 2173196 | + |
| mir-35 | II | 11537400 | 11538340 | + |
| mir-54 | X | 13144621 | 13145200 | − |
| mir-58 | IV | 3232800 | 3233347 | + |
| mir-61 | V | 11769899 | 11770300 | − |

Definition of Unique 21U-RNAs

A custom PERL script was used to identify all annotated 21U-RNAs which were mapped to unique genomic loci without overlapping with each other. A genomic locus is defined as a combination of chromosome, strand and start position. The total number of such 21U-RNA is 9079 out of 15073 in WS215.

Identification of CapSeq Reads Overlapping with 21U-RNAs

Uniquely matched reads without mutation at the 1^{st} nt were overlapped with the unique 21U-RNAs. Included were the CapSeq reads overlapping with only one 21U-RNA on the same strand. The relative position of the 5′ end of CapSeq read was referred to the 5′ end (+1) of the 21U-RNA overlapped, and a negative number represented upstream. For the CapSeq reads starting 2 nt upstream the annotated 21U-RNAs, annotated non-21U-RNA genes were searched within 20 nt upstream to 200 nt downstream the CapSeq reads.

Analysis of csRNAs Overlapping with 21U-RNAs

The RNAs overlapping with only one unique 21U-RNA on the same strand were analyzed using CIP-TAP and CIP-PNK samples. To analyze the correlation of 21U-RNA and csRNA, the reads mapped to each unique 21U-RNA were obtained from CIP-PNK and CIP-TAP samples respectively. Only included were reads mapped to the starting positions of 21U-RNAs in CIP-PNK and those mapped 2 nt upstream the annotated starting positions of 21U-RNAs in CIP-TAP. The reads were normalized to 5 million of non-structural reads. To draw the figure using log 2 scale, we only considered 21U-RNA loci with at least 1 reads in either CIP-PNK or CIP-TAP sample. Furthermore, if one of the two numbers was less than 1, we assigned that number to 1 to avoid drawing negative number using log 2 scale.

Identification of 21U-Like RNA Loci

To identify loci similar to 21U-RNAs but failing to generate 21U-RNAs, we used CIP-TAP sample with restraints either from inside or outside. Included in this analysis were the reads uniquely mapped and located on chromosome IV within the two intervals of 4500000-7000000 and 13500000-17200000. The inside filter was to filter out sense RNA reads mapped to 21U-RNA loci, anti-sense and sense reads mapped to miRNA/tRNA/rRNA loci, and anti-sense reads mapped to protein coding genes and pseudogenes. The outside restraints included PRG-1 IP and CIP-PNK. Any read enriched 5× or more in the PRG-1 IP, as compared to the input, was considered as a 21U-RNA candidate and was removed from CIP-TAP sample. However, we cannot directly filter out these PRG-1 IP RNA loci, represented by the start site, because 21U-RNAs were usually located 2 nt downstream the potential csRNAs cloned in CIP-TAP sample. Therefore these IP loci were shifted 2nt upstream first, and then used as a filter. CIP-PNK sample was used in two ways to remove potential noises. First, CIP-PNK sample served as control for CIP-TAP, and included in the analysis were RNAs in the CIP-TAP sample with at least 1 read/million of non-structural reads and enriched at least 10 fold as compared to CIP-PNK sample. Second, CIP-PNK sample was used to filter out mapped loci in CIP-TAP near which there were at least two other loci in CIP-PNK, because 22G-RNAs, the major background noise in CIP-TAP sample, were usually overlapped with each other. The nearby regions were defined as the regions from position −25 to −5 for upstream and +6 to 26 for downstream the start sites of the mapped reads in CIP-TAP. The motif and size analysis was performed using the −2,300 loci obtained this way, and the position in the motif analysis was referred to the start sites of csRNAs.

Identification of Novel 21U-RNAs in Wild Isolates

A custom PERL script searched 21U-RNA candidates cloned in the wild isolates using criteria: 1) the 21U-RNA locus had a ‘U’ at +1 position in the wild isolates but other nucleotides in N2, as annotated in WS215; 2) the 21U-RNA has YR motif; 3) 21U-RNA had at least 5 reads after being normalized to 5 million of non-structural reads, and was mapped to a unique locus; 4) the 21U-RNA was mapped to chromosome IV within the two intervals of 4500000-7000000 or 13500000-17200000.

Identification of New 21U-RNAs

New 21U-RNAs were defined using PRG-1 IP with the inside restraints and outside restraints. The inside restraints removed reads mapped to tRNAs or rRNAs, RNAs of non-20/21-U, and RNAs with mutation at the 1^{st} position or with less than 1 read per 5 million nonstructural reads in PRG-1 IP. The outside restraints remove the reads without a YR motif in which Y is 3 nt upstream the RNA start sites, and the reads enriched less than 5 fold, as compared to the input sample. csRNAs were defined as the mapped RNA loci enriched at least 10 fold in the CIP-TAP sample over CIP-PNK sample.

Analysis of the Percentage of Non-Canonical 21 U-RNAs

This analysis was different from analysis 18 above because the new 21U-RNAs defined there could contain many canonical 21U-RNAs missed in the published list due to insufficient sequencing depth. In this analysis, such 21U-RNAs were removed. Each small RNA sample was normalized to 5 million of non-structural reads, and Capseq YA0831 was normalized to 10 million of sense protein coding reads. U-21nt RNA, starting with U and 21 nt long, was obtained as the RNA reads uniquely matched and enriched at least 5 fold in CIP-TAP sample, as compared to CIP-PNK sample. rRNAs and tRNAs were removed beforehand. These RNAs are divided into 4 groups: 1) mapped within the two regions of 4500000-7000000 and 13500000-17200000 on chromosome IV and annotated as 21U-RNAs already; 2) mapped within the two regions of 4500000-7000000 and 13500000-17200000 on chromosome IV, but not annotated as 21U-RNAs; 3) mapped to other regions on chromosome IV or other chromosomes, but annotated as 21U-RNAs; 4) mapped to other regions on chromosome IV or other chromosomes, but not annotated as 21U-RNAs. Group 2 and group 4 U-21nt RNAs must have YR motif. Group 1 represented the canonical 21U-RNAs, while group 4 represented the non-canonical 21U-RNAs. Non-SL-containing CapSeq RNAs in YA0831 and csRNAs enriched in CIP-TAP sample were searched for overlapping with U-21nt RNAs in group 1 and group 4. csRNAs were RNAs uniquely matched and enriched at least 5 fold in CIP-TAP sample, as compared to CIP-PNK sample. CapSeq RNAs were RNAs uniquely matched, containing no SL sequence, and perfectly matched at the first nucleotide in YA0831. Both csRNAs and CapSeq RNAs started 2 nt upstream U-21nt RNAs in either group 1 or group 4.

Mouse Capseq Analysis

Genome and annotations NCBIM37.67 were obtained from ftp.ensembl.org, CAGE data was obtained from http://fantom31p.gsc.riken.jp/cage/download/mm5/, non-coding RNA database fRNAdb v3.4 was obtained from http://www.ncrna.org/frnadb/, and miRNA database miRBase release 18 was obtained from miRBase. These annotations were mapped to mouse genome using a custom PERL script plus Bowtie. Bowtie was also used to map reads of >=19 nt long to the genome and annotations with parameters: -n 2 -e 180 -a --best --strata -m 200. A more stringent mutation filter was used to exclude non-specific matches: 0 mismatch for reads 19-24 nt long, 1 for 25-29 nt, 2 for 30-39 nt, 3 for 40-49nt, 4 for 50-59 nt, 5 for 60-69, and 6 for >=70 nt. A custom PERL script was used to obtain the start locus histogram of the mapped reads, after being normalized to 10 million of non-structural RNA reads. And all the alignments were visualized using Gbrowse 1.70.

Mapping Small RNAs to Mouse Genome

The mouse Mili IP data was obtained from GEO GSM475280 (Robine et al., Curr Biol, 19: 2066-2076, 2009), and then reads of size >=18 nt was mapped with Bowtie 0.12.7, using parameters ‘-n 2 -e 180-a--best-strata -m 200’. The mutation rate allowed was as described above for mouse CapSeq analysis. The post-bowtie analysis was the same as for the C. elegans small RNA.

Analysis of the Mouse CapSeq Quality—the Rate of Capped RNA

The range of the potential start site for each transcript annotated was defined as the region from position −20 to 21 relative to +1, the annotated start position. To account for alternative splicing, which could annotate the same state sites on one transcript as the non-start sites on the other transcript, the start sites had priority over non-start sites, thus being removed from the non-start sites. The total number of genomic positions for each class of sites, either start or non-start, and the reads matched, were used to estimate the overall enrichment for capped RNAs.

Motif Analysis of Mouse CapSeq and CAGE

Included in the analysis were RNA reads uniquely matched without any mismatch at the first position, after removal of RNAs mapped to tRNA, rRNA, snRNA and snoRNA. A histogram of the RNA start sites was made. The start sites analyzed had at least 1 reads out of 1 million of non-structural RNA reads, and there were no other start sites 1nt upstream or downstream with 10 fold more reads. The nucleotide occurrence around each start site (−50 to 50) was used to calculate the overall frequency table. To avoid bias from the start sites with the abundant reads, the weight for each start site was set to 1. The log₂ ratio of foreground rate/background rate represented the relative enrichment for each nucleotide at each position. Here the background rate for A/G/C/U was based on the nucleotide frequency of all genomic regions from position −200 to +200 relative to each start site, and the foreground frequency for each nucleotide at each position was calculated using the −50 to 50 frequency table above.

For the CAGE data, if the start position of the RNA had a mismatch, the start position was reset as the position +2, as long as this position was perfectly matched. Or the RNA was discarded.

Prediction of TS Sites for Mouse Pri-miRNAs

Mouse testis samples at 4 weeks and 6 months were used in this analysis. Reads mapped to more than 3 genomic loci, or mapped with mutation at position +1, or mapped to snRNA, snoRNA, rRNA, tRNA, mRNA and piRNA were excluded. Reads of different length were combined according to the start sites, and then the start sites with less than 1 read per 5 million non-structural RNA reads were excluded. Also excluded were the start sites with less than 1/10^{th} reads of the flanking start sites either 1nt upstream or downstream. The start sites were then assigned to the annotated pre-miRNAs as long as the start sites are within 1 kb upstream of the pre-miRNAs. Totally, 134 pre-miRNAs were assigned with TS sites, and these TS sites were enriched for YR motif (data now shown.)

1. A method of enriching for capped RNA present in a starting RNA sample containing rRNA comprising:
a) contacting a starting RNA sample 5′ monophosphate-dependent exonuclease to obtain a population of 5′ monophosphate-dependent exonuclease resistant RNAs depleted of 28S, 16S, and 5.8S rRNA;
b) contacting the population of 5′ monophosphate-dependent exonuclease resistant RNAs with at least one phosphatase to obtain a population of phosphatase-resistant RNAs depleted of RNAs having exposed 5′ phosphate groups; and
c) contacting the population of phosphatase-resistant RNAs with a decapping enzyme to obtain a population of RNAs having an exposed alpha-phosphate in the 5′ Gppp cap,
to thereby enrich for capped RNA present in a starting RNA sample containing rRNA.

2. The method of claim 1, wherein the starting RNA sample is total cellular RNA.

3. The method of claim 1, wherein the starting RNA sample comprises 500 ng or less of RNA.

4. The method of claim 1, wherein the starting RNA sample includes polyA+ and polyA-RNA.

5. The method of claim 1, wherein the starting RNA sample comprises degraded RNA.

6. The method of claim 1, further comprising sequencing the population of RNAs having an exposed alpha-phosphate in the 5′ Gppp cap.

7. The method of claim 1, further comprising the step of contacting the population of RNAs having an exposed alpha-phosphate in the cap with an RNA ligase to obtain a population of ligated RNAs.

8. The method of claim 7, further comprising subjecting the population of ligated RNAs to RT PCR using random primers to obtain a cDNA library.

9. The method of claim 8, further comprising subjecting the cDNA library to a size selection procedure.

10. The method of claim 8, further comprising amplifying the cDNA library by PCR.

11. The method of claim 5, the method comprising treating the starting RNA sample with a polynucleotide kinase prior to step a) to phosphorylate the 5′ terminus of degraded RNA.

12. The method of claim 8, further comprising sequencing 100 bases or fewer of the cDNA members of the library.

13. The method of claim 8, further comprising sequencing between 50 and 125 bases of the cDNA members of the library.

14. A composition obtained using the method of claim 1.

15. An RNA mixture comprising RNA molecules comprising transcriptional start sites, wherein the RNA molecules comprise monophosphorylated 5′ termini or hydroxyl 5′ termini, wherein the mixture is substantially free of 28S, 16S, and 5.8S ribosomal RNA, RNA with triphosphorylated 5′ termini, and 5′ m7G capped RNA.

16. The RNA mixture of claim 15, wherein the mixture comprises RNAs substantially free of poly-A at the 3′ terminus.

17. The RNA mixture of claim 15, wherein the mixture is substantially free of RNA with poly-A at the 3′ terminus.

18. The RNA mixture of claim 15, wherein the mixture comprises RNA with a size of less than 200 nucleotides.

19. The RNA mixture of claim 15, wherein the mixture comprises RNA with a size of between about 50 nucleotides and about 200 nucleotides.

20. The RNA mixture of claim 15, wherein the RNA mixture comprises a substantially pure population of RNA molecules having a size of between about 130 nucleotides and 170 nucleotides.

21. A cDNA library generated from the RNA mixture of claim 15.

22. A kit comprising: a first component comprising a 5′ monophosphate-dependent exonuclease, a second component comprising a phosphatase, and a third component comprising a decapping enzyme.

23. The kit of claim 22, further comprising a control sample.

24. The kit of claim 22, further comprising instructions for use of the kit to enrich for capped RNAs.

25. A method of identifying a candidate transcriptional start (TS) site, the method comprising:
(a) obtaining a 5′ sequence tag from a cDNA library generated according to the method of claim 8;
(b) mapping the 5′ sequence tag to genes within a database of gene sequences; and
(c) identifying a candidate TS site as one occurring at or near the site to which the 5′ sequence tag maps.

26. The method of claim 25, wherein the site to which the 5′ sequence tag maps overlaps with the 5′ nucleotide sequence of a gene; is within 100 nucleotides upstream of a gene, or is within 500 nucleotides upstream of a gene.

27. The method of claim 25, wherein the cDNA library is generated from an RNA sample selected from the group consisting of a cell-type specific sample, a development stage-specific sample, an organism-specific sample, a tissue-specific sample, and a disease-specific sample.

28. The method of claim 25, wherein the database of gene sequences is a whole genome database, a miRNA database, an organ-specific database, a tissue-specific database, or a database of non-coding RNAs.


SEQUENCE LISTING (18 sequences)

SEQ ID NO: 1 - DNA, 279 bp, C. elegans
gtgtcggccgccgccaaacacctactcctgcatcgttctgtgtgttttaatgtgttttttgttgaggtatctttatatcaataatgaataatgtcgccaccccatagaaatattttctatgtcaacctcttgcacggttgccgtagttaatttttctttgggaataggctaaagtgaagcgttgcggtcactctggatcatgactttctaccgaaaaaatttttgaaacaggctattctattttattaggctctgcttttcatgtgaggatggaaatat

SEQ ID NO: 2 - DNA, 280 bp, C. elegans
gtgtcgcccgccgacaaacacctaccccttcgtcgttctttgtgttttgatgtgatcataactataagaataataataatgaataatgtcgcaatcccatagaaatattttctatgtcaacctcttgtacggttgctgtagttaattttttctttgggaataggcgaaaatgacgctttgcggtcactcaaaatcatgattttctaggggaaaaattatataaatttcggaactaaattatttcattagattcttcttttttatgtgaggctgaaaatat

SEQ ID NO: 3 - DNA, 284 bp, C. elegans
gtgtcggccaccgccaaacacctactctttcaacgttctgtgtgttttgatgcgatttcaaattttgcccattcaatataataatgaaaaatgtcgccatcccatagaaatattttctatgccaacctcttgtacggttgccgtagttagttttttctttgggaataggcgaaagtgacgcgtttcggtcactcaaaatcatgattttctgggggaaaaattataaaactttaggaaataaattatttcattaggttcttcttcttatgtgaggctgaaaatat

SEQ ID NO: 4 - DNA, 278 bp, C. elegans
gtgtcggcggccaccaaacacctactactccgctgatctgtgttctttgatgcaatttaactatatgactattataataatgaaaaatgtcgccatcctatagaaaaaatgttccatgtcaacctcttccacggttgccgtaattaattttctctttgggaatatgcgaaagtgatgcgttgcggtcactcagaatcatgattttctagcggaaaaattataaaatttcggaactattttactttatcaggtttttgttcgtgtaagggtaaaaatat

SEQ ID NO: 5 - DNA, 270 bp, C. elegans
gtgtcggcggccgccgaacacctacccctttatcgatctgtgttctttgaagtgatatgtttatatccatgaatctataataatgaataatatcgccatcccatagaaatattttctatgtcaacctctacaacggttgccgtagttaaattttttctttgggaatttgcgaaagtgatttgttgcggtcactcagaattatgattttactagcaaagaaagtccaaattgaaagctattttggaaattttatggtttgctttgagttat

SEQ ID NO: 6 - DNA, 284 bp, C. elegans
gttgtcggcggccttcaaacacctactccttctttgattcgtgttctttgatgtgatttgactatatccattaatattaaaaaatgaataatgtcgccatcctatagaaataatgttccatgtcaacctctttcacggttgccatagttaatttttttctttgggattttgcgaaagtgattcgttgcggtcactcagtatcatgatttagccatatttaggaacgaataattctgaccaaatagcccgtacaaaattactacaacgtataatcccaacaagat

SEQ ID NO: 7 - DNA, 25 bp, Artificial Sequence
  Synthetic oligonucleotide
cagaagacggcatacgannnnnnnn

SEQ ID NO: 8 - DNA, 21 bp, Artificial Sequence
  Synthetic oligonucleotide
caagcagaagacggcatacga

SEQ ID NO: 9 - DNA, 19 bp, Artificial Sequence
  Synthetic oligonucleotide
gttctacagtccgacgatc

SEQ ID NO: 10 - DNA, 49 bp, Artificial Sequence
  Synthetic oligonucleotide
aatgatacggcgaccaccgacaggttcagagttctacagtccgacgatc

SEQ ID NO: 11 - DNA, 16 bp, Artificial Sequence
  Synthetic oligonucleotide
gtaaaacgacggccag

SEQ ID NO: 12 - DNA, 17 bp, Artificial Sequence
  Synthetic oligonucleotide
caggaaacagctatgac

SEQ ID NO: 13 - DNA, 20 bp, Artificial Sequence
  Synthetic oligonucleotide
taatacgactcactataggg

SEQ ID NO: 14 - DNA, 17 bp, Artificial Sequence
  Synthetic DNA/RNA hybrid oligonucleotide
tctacaguccgacgauc

SEQ ID NO: 15 - DNA, 18 bp, Artificial Sequence
  Synthetic oligonucleotide
gcagaagacggcatacga

SEQ ID NO: 16 - DNA, 32 bp, Artificial Sequence
  Synthetic oligonucleotide
aacaattttatttatgctgcgacacgatttcg

SEQ ID NO: 17 - DNA, 36 bp, Artificial Sequence
  Synthetic oligonucleotide
agagatttttcctatttctcctgttctcaacgcaaa

SEQ ID NO: 18 - DNA, 26 bp, Artificial Sequence
  Synthetic oligonucleotide
tttaatgtgttttttgttgaggtatc